(19) United States
(12) Patent Application Publication
(10) Pub. No.: US 2020/0138964 A1
(43) Pub. Date: May 7, 2020

(54) TERTIARY AMINE COMPOUND OR IMINE COMPOUND-POLYMER CONJUGATE AND PRODUCTION METHOD THEREFOR

(71) Applicant: Seikagaku Corporation, Tokyo (JP)

(72) Inventors: Nobuo Kobayashi, Tokyo (JP); Yosuke Yasuda, Tokyo (JP); Kenichi Namatsu, Tokyo (JP)

(73) Assignee: Seikagaku Corporation, Tokyo (JP)

(21) Appl. No.: 16/608,677

(22) PCT Filed: Apr. 25, 2018
(86) PCT No.: PCT/JP2018/016843

§ 371 Date: Oct. 25, 2019

(30) Foreign Application Priority Data
Apr. 25, 2017 (JP) 2017-086223

(51) Int. Cl. A61K 47/61; C08B 37/00; A61K 47/62; A61K 47/58; A61K 31/4178; A61K 31/452; A61K 31/5415; C08B 37/08; C08B 11/145; C08G 69/48; C08F 8/30

(52) U.S. Cl. CPC A61K 47/61; C08B 37/0069; A61K 47/62; A61K 47/58; A61K 31/4178; C08F 8/30; A61K 31/5415; C08B 37/0072; C08B 11/145; C08B 37/0084; C08G 69/48; A61K 31/452

(57) ABSTRACT

Provided is a compound obtained by conjugating a tertiary amine compound or imine compound, which is useful as a drug, with a polymer, in which a structure D⁺ having a quaternary ammonium salt or iminium salt formed from a tertiary amine compound or imine compound D and a polymer residue Poly having a carboxy group are bonded to each other via a structure —C(R¹)(R²)OC(O)ANHC(═O)—.

### TECHNICAL FIELD

The present invention relates to a novel conjugate of a tertiary amine compound or an imine compound with a polymer and a production method therefor. Specifically, the present invention relates to a novel conjugate of a tertiary amine compound or an imine compound with a polymer using, as a linker, an aminoacyloxymethyl group whose release rate can be controlled.

### BACKGROUND ART

A conjugate of a drug with a polymer has been widely reviewed in a field of a prodrug or drug delivery system (DDS), and is an important means for providing a function such as release control, absorption improvement, stabilization in a living body, or targeting to a target tissue.

For example, a conjugate using polyglutamic acid that is one of polyamino acids has been reported in JP 2003-511423 A. A conjugate with gossypol using carboxymethyl cellulose (CMC) that is used as a medical drug additive has been reported in JP 5690944 B2. Alginic acid that is one of dietary fibers in polysaccharides has also been reviewed, and conjugates with various drugs have been reported in JP H08-24325 A. Further, glycosaminoglycan of natural polysaccharide has also been widely reviewed, and a conjugate of hyaluronic acid or chondroitin sulfate with a peptide has been reported in U.S. Pat. No. 5,955,578. In addition, a conjugate using heparin has been reported in WO 1993/18793 A. Moreover, also regarding a conjugate using hyaluronic acid, application in the field of joint diseases (WO 2005/085294 A) and a conjugate with a anticancer drug have been also reviewed (JP 2006-504747 A).

Meanwhile, a method of conjugating a polymer with a drug is roughly classified into two types: 1) a method of directly bonding a polymer to a drug (JP 2006-504747 A) and 2) a method of bonding a polymer to a drug via a linker (JP 2003-511423 A).

When a structure of a drug to be bonded to and conjugated with a polymer is confirmed, a drug having an amino group, a carboxy group, or a hydroxyl group as a functional group in the molecule is utilized. In a bonding mode thereof, regarding a primary or secondary amino group drug, a method of bonding by reductive amination with a primary amino group drug (JP 2000-501082 A) and a method of forming an amide bond with a primary or secondary amino group drug (JP H08-24325 A) have been known.

### CITATION LIST

### Patent Literature

### Patent Literature 1: JP 2003-511423 A

### Patent Literature 2: JP 5690944 B2

### Patent Literature 3: JP H08-24325 A

Patent Literature 4: U.S. Pat. No. 5,955,578

### Patent Literature 5: WO 1993/18793 A

### Patent Literature 6: WO 2005/085294 A

### Patent Literature 7: JP 2006-504747 A

### Patent Literature 8: JP 2000-501082 A

### SUMMARY OF INVENTION

### Technical Problem

A carboxy group polymer is a very attractive carrier, but conjugation of an active compound has hitherto been realized as long as the active compound has a primary or secondary amino group, a carboxy group, or a hydroxyl group as a functional group. Meanwhile, a tertiary amine compound or imine compound which is useful as a drug exists in large numbers, but a conjugate with a tertiary amine compound or an imine compound with a polymer has not been known. Since conjugation reaction is selected depending on a functional group of a drug, a conjugate with a tertiary amine compound or an imine compound cannot be obtained by a method of the related art, and thus construction of a novel method has been desired. Further, it is preferable that a conjugate releases a drug in a living body, and searching for a polymer or linker which is suitable for conjugation with a tertiary amine compound or an imine compound has also been required.

An object of the present invention is to provide a novel conjugate of a tertiary amine compound or an imine compound with a polymer having a carboxy group and a production method therefor.

The present inventors have conducted intensive studies on a linker capable of forming a conjugate of a tertiary amine compound or an imine compound with a polymer having a carboxy group, and as a result, have found an aminoacyloxymethyl group linker whose release rate can be controlled. The present invention is based on the finding of a linker which has not existed hitherto and by which a tertiary amine compound or an imine compound can be bonded to a polymer having a carboxy group in the form in which a release rate can be controlled, and relates to a novel tertiary amine compound or imine compound-polymer conjugate and a production method therefor.

The present invention relates to inventions specified by the following items.

1. A compound represented by Formula (I) or a pharmaceutically acceptable salt thereof;

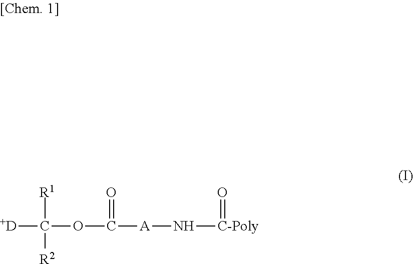

in Formula (I), D⁺ is a structure forming a quaternary ammonium salt or an iminium salt formed from D, a tertiary amine compound or an imine compound; a nitrogen atom forming the quaternary ammonium salt or the iminium salt and a carbon atom to which R¹ and R² bond are bonded to each other, R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a bivalent hydrocarbon group in which a carbon other than carbons at both ends may be substituted with a hetero atom selected from the group consisting of an oxygen atom, a nitrogen atom and a sulfur atom; any two or three groups of R¹, R², and A may combine together to form a ring; and Poly is a polymer residue having a carboxy group.

2. A compound represented by Formula (II) or a pharmaceutically acceptable salt thereof;

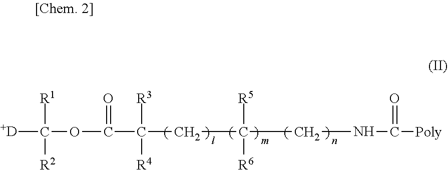

in Formula (II), D⁺, R¹, R², and Poly are as defined above; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring; 1 and n are independently 0, 1, or 2; and m is 0 or 1.

3. The compound according to 1. or 2. or a pharmaceutically acceptable salt thereof, wherein in Formula (I) or (II); R¹, R², R³, R⁴, R⁵, and R⁶ are each independently a hydrogen atom, a substituted or unsubstituted linear or branched chain alkyl group having carbon number of 1 to 6, a substituted or unsubstituted cycloalkyl group having carbon number of 3 to 8, a substituted or unsubstituted linear or branched alkenyl group having carbon number of 2 to 6, a substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom.
4. The compound according to in any one of 1. to 3. or a pharmaceutically acceptable salt thereof, wherein in Formula (I) or (II), a substituent of alkyl, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁶ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group, Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group, and at this time, two or more of Rx, Ry, and Rz may be combined together to form a saturated or unsaturated hetero ring.
5. The compound according to any one of 1. to 4. or a pharmaceutically acceptable salt thereof, wherein in Formula (I) or (II), Poly is a water-soluble polymer residue.
6. The compound according to any one of 1. to 4. or a pharmaceutically acceptable salt thereof, wherein in Formula (I) or (II), Poly is a polysaccharide residue.
7. The compound according to any one of 1. to 4. or a pharmaceutically acceptable salt thereof, wherein in Formula (I) or (II), Poly is a glycosaminoglycan residue.
8. The compound according to any one of 1. to 4. or a pharmaceutically acceptable salt thereof, wherein in Formula (I) or (II), Poly is a residue of chondroitin, chondroitin sulfate or hyaluronic acid
9. A method for producing a compound represented by the following Formula (I) or a pharmaceutically acceptable salt thereof, the method including a step of condensing a compound represented by the following Formula (III) and a polymer having a carboxy group represented by the following Formula (IV):

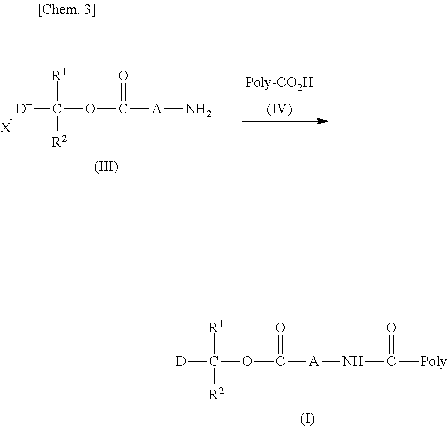

X⁻ is a counter anion of D⁺, and the compound represented by Formula (III) may form a salt with an inorganic acid or an organic acid.
10. The production method according to 9, wherein the compound represented by Formula (III) is a compound represented by the following Formula (IX), and the compound represented by Formula (I) is a compound represented by the following Formula (II):

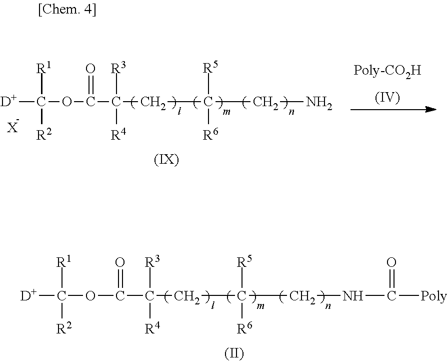

in Formulae (TI), (IV), and (IX), D⁺, R¹, R², R³, R⁴, R⁵, R⁶, l, n, m, and Poly are as defined above, X⁻ represents a counter anion of D⁺, and the compound represented by Formula (IX) may form a salt with an inorganic acid or an organic acid.

11. A linker represented by the following Formula (V) for bonding a tertiary amine compound containing a nitrogen atom capable of forming a quaternary ammonium salt or an imine compound capable of forming an iminium salt to a polymer having a carboxy group:

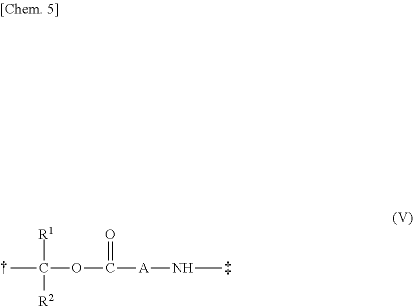

in which, R¹, R², and A in the above (V) are as defined above, symbol † is a node with the nitrogen atom forming the quaternary ammonium salt or the iminium salt, and symbol ‡ represents a node with a moiety of the carboxy group excluding a hydroxyl group of the polymer having a carboxy group.

12. The linker according to 11, in which the linker is represented by the following Formula (XV):

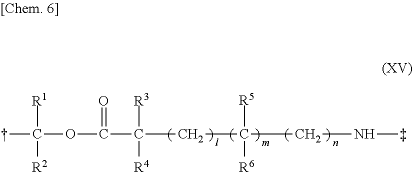

in which, R¹, R², R³, R⁴, R⁵, R⁶, l, m, and n in the above (XV) are as defined above; symbol † is a node with the nitrogen atom forming the quaternary ammonium salt or the iminium salt, and symbol ‡ represents a node with a moiety of the carboxy group excluding a hydroxyl group of the polymer having a carboxy group.

13. A method for producing a conjugate, the method including a step of bonding a tertiary amine compound containing a nitrogen atom capable of forming a quaternary ammonium salt or an imine compound capable of forming an iminium salt to a polymer having a carboxy group via the linker according to 11. or 12.

### BRIEF DESCRIPTION OF DRAWINGS

FIG. 1 is a graph showing a relation between time and a drug release ratio in a buffer solution having a pH of 7.0 in Examples 4, 20, 24, 30, and 38.

FIG. 2 is a graph showing a relation between time and a drug release ratio in a buffer solution having a pH of 7.0 in Examples 2, 8, 18, 22, and 26.

FIG. 3 is a graph showing a relation between time and a drug release ratio in a buffer solution having a pH of 7.0 in Examples 6, 10, and 43 to 45.

FIG. 4 is a graph showing a relation between time and a drug release ratio in a buffer solution having a pH of 7.0 in Example 32.

FIG. 5 is a graph showing a relation between time and a drug release ratio in a buffer solution having a pH of 7.0 in Examples 34 and 36.

### DESCRIPTION OF EMBODIMENTS

A conjugate according to an aspect of the present invention is a compound having a structure represented by the following Formula (I) or a pharmaceutically acceptable salt thereof.

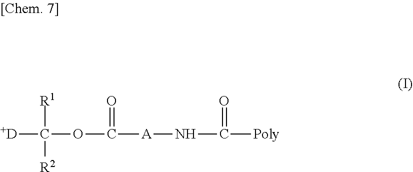

[In the formula, D⁺, R¹, R², A, and Poly are as defined above].

A structure derived from a polymer having a carboxy group which is represented by Poly and a structure having a quaternary ammonium salt or iminium salt formed from a tertiary amine compound or an imine compound D are bonded to each other via a linker sandwiching a hydrocarbon group A to form a conjugate. The conjugate is preferably a conjugate with a drug containing a tertiary amine or imine structure.

In a bioactive substance including a medical drug, extremely many compounds having tertiary amine or imine exist, but in the techniques of the related art, there is no means for bonding those compounds to a polymer having a carboxy group in the form in which a release rate can be controlled. The linker having the structure found in the present invention enables a conjugate of a tertiary amine compound or imine compound with a polymer having a carboxy group which cannot be prepared hitherto to be produced and considerably contributes to medical treatment and the like.

The conjugate bonds to a hydrocarbon chain of the linker when the carboxy group of the polymer residue forms an amide bond. The bivalent hydrocarbon group represented by A in the above Formula (I) may be a carbon chain having carbon number of not less than 1 or may have a branched structure or a cyclic structure. In a case where the number of carbon atoms is 3 or more, a carbon atom other than carbon atom at both ends may be substituted by a hetero atom selected from the group consisting of a nitrogen atom, an oxygen atom, and a sulfur atom. Further, R¹ and/or R² can also be combined together to form a ring. A preferably represents a bivalent hydrocarbon group represented by C(R³)(R⁴)—(CH₂)ₗ—(C(R⁵)(R⁶))ₘ—(CH₂) as represented by the following Formula (II) (herein, R³ to R⁶, l, and m, n are as defined above). From the viewpoint of ease of design and availability of a raw material, A preferably represents a linear or branched alkylene group having carbon number of 1 to 10, and the carbon number of A is further preferably 1 to 6.

The terminal at the opposite side of the amide bond of the hydrocarbon group is bonded to a substituted or unsubstituted methylene group represented by —C(R¹)(R²)— in the above Formula (I) via an ester bond. The methylene group forms bonds in the order of an oxygen atom of an ester bond-the methylene group-a nitrogen atom of a quaternary ammonium salt or an iminium salt in Formula (I). The methylene group may be unsubstituted or substituted and may be bonded to the bivalent hydrocarbon group to form a ring. The tertiary amine compound or imine compound exists in the structure of the conjugate as the quaternary ammonium salt or iminium salt via the linker.

The structure D⁺, which has formed a quaternary ammonium salt or iminium salt, at the terminal of the conjugate can rapidly release the tertiary amine compound or imine compound D owing to the presence of an oxymethylene group to be bonded to the structure D⁺. This mechanism will be described using the compound represented by Formula (I) as follows. Regarding the tertiary amine compound or imine compound-polymer conjugate represented by Formula (I), an ester bond moiety is hydrolyzed in the presence of water to be decomposed into a hydroxymethyl form represented by Formula (VI) and a carboxylate form represented by Formula (VII). Further, the hydroxymethyl form represented by Formula (VI) is unstable in terms of the structure since the hydroxymethyl form has a quaternary ammonium salt or iminium salt structure, and at the same time of generating the hydroxymethyl form, the hydroxymethyl form is decomposed into the tertiary amine compound or imine compound D and an aldehyde form (or a ketone form) represented by Formula (VIII). At this time, the function of the generated tertiary amine compound or imine compound is exhibited. Therefore, the tertiary amine compound or imine compound-polymer conjugate represented by Formula (I) can control releasing of the tertiary amine compound or imine compound by controlling a hydrolysis rate of the ester bond moiety, so that sustainability of the function of the tertiary amine compound or imine compound can be controlled.

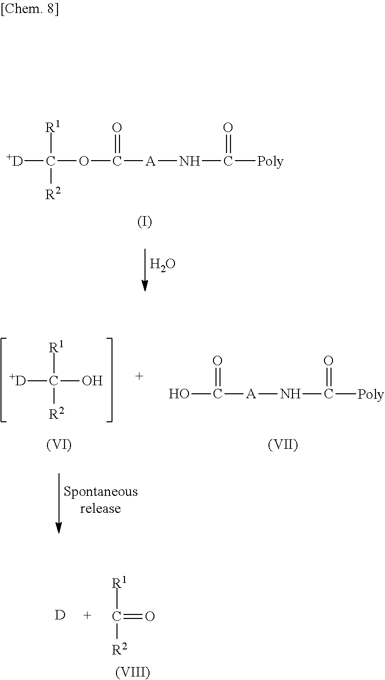

One embodiment of the tertiary amine compound or imine compound-polymer conjugate of the present invention is the compound represented by the above Formula (I) or (II), and the amine form that is an important intermediate of the compound represented by (I) or (II) is a compound represented by the following Formula (III) or (IX):

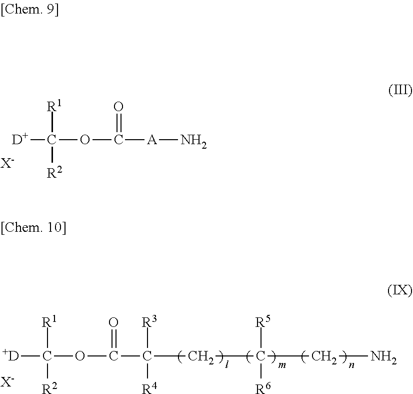

[in Formula (III) or (IX), D⁺, R¹, R², R³, R⁴, R⁵, R⁶, A, l, m, and n are as defined above, and X⁻ represents a counter anion of the quaternary ammonium salt or iminium salt in D⁺]. The compound represented by the above Formula (III) or (IX) may further form a salt with an inorganic acid or an organic acid.

Specific examples of the alkyl group, the cycloalkyl group, the alkenyl group, the cycloalkenyl group, the alkynyl group, the aromatic group, and the heterocyclic group included in the groups represented by the substituent R¹, R², R³, R⁴, R⁵, and R⁶ in Formulae (I), (II), (III), and (IX) include the following groups.

As the alkyl group, any of a linear or branched chain alkyl group may be used, and the number of carbon atoms is preferably 1, 2, 3, 4, 5, or 6. Examples of the alkyl group may include a methyl group, an ethyl group, a n-propyl group, a 2-propyl, a n-butyl group, a 1-methylpropyl group, a 1,1-dimethylethyl group, a 2-methylpropyl group, a n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a n-hexyl group, a 1-methylpentyl group, a 2-methylpentyl group, a 3-methylpentyl group, a 4-methylpentyl group, a 1-ethylbutyl group, a 2-ethylbutyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1,1,2-trimethylpropyl group, a 1-ethyl-1-methylpropyl group, and a 1-ethyl-2-methylpropyl group.

Any cycloalkyl group may be used as long as the carbon atom at the node is included as an atom configuring a ring, the cycloalkyl group may be condensed with cycloalkane, cycloalkene, an aromatic ring, or a hetero ring, and may form a spiro ring, and the number of carbon atoms is preferably 3, 4, 5, 6, 7, or 8. Examples of the cycloalkyl group may include a cyclopropyl group, a cyclobutyl group, a cyclopentyl group, a cyclohexyl group, a cycloheptyl group, and a cyclooctyl group.

As the alkenyl group, any of a linear, branched, or cyclic alkenyl group may be used, and the number of carbon atoms is preferably 2, 3, 4, 5, or 6. Examples of the alkenyl group may include a vinyl group, a 1-propenyl group, a 2-propenyl group, a 1-methylvinyl group, a 1-butenyl group, a 2-butenyl group, a 3-butenyl group, a 1-ethylvinyl group, a 1-methyl-1-propenyl group, a 1-methyl-2-propenyl group, a 2-methyl-1-propenyl group, a 2-methyl-2-propenyl group, a 1-pentenyl group, a 2-pentenyl group, a 3-pentenyl group, a 4-pentenyl group, a 1-propylvinyl group, a 1-methyl-1-butenyl group, a 1-methyl-2-butenyl group, a 1-methyl-3-butenyl group, a 2-methyl-1-butenyl group, a 2-methyl-2-butenyl group, a 2-methyl-3-butenyl group, a 3-methyl-1-butenyl group, a 3-methyl-2-butenyl group, a 3-methyl-3-butenyl group, a 1-ethyl-1-propenyl group, a 1-ethyl-2-propenyl group, a 1-(2-methylethyl)vinyl group, a 1,2-dimethyl-1-propenyl group, a 1,2-dimethyl-2-propenyl group, a 1,1-dimethyl-2-propenyl group, a 1-hexenyl group, a 2-hexenyl group, a 3-hexenyl group, a 4-hexenyl group, a 5-hexenyl group, a 1-butyl vinyl group, a 1-methyl-1-pentenyl group, a 1-methyl-2-pentenyl group, a 1-methyl-3-pentenyl group, a 1-methyl-4-pentenyl group, a 2-methyl-1-pentenyl group, a 2-methyl-2-pentenyl group, a 2-methyl-3-pentenyl group, a 2-methyl-4-pentenyl group, a 3-methyl-1-pentenyl group, a 3-methyl-2-pentenyl group, a 3-methyl-3-pentenyl group, a 3-methyl-4-pentenyl group, a 4-methyl-1-pentenyl group, a 4-methyl-2-pentenyl group, a 4-methyl-3-pentenyl group, a 4-methyl-4-pentenyl group, a 1-propyl-1-propenyl group, a 1-propyl-2-propenyl group, a 1-ethyl-1-butenyl group, a 1-ethyl-2-butenyl group, a 1-ethyl-3-butenyl group, a 2-ethyl-1-butenyl group, a 2-ethyl-2-butenyl group, a 2-ethyl-3-butenyl group, a 1-(2-methylpropyl)vinyl group, a 1,2-dimethyl-1-butenyl group, a 1,2-dimethyl-2-butenyl group, a 1,2-dimethyl-3-butenyl group, a 1-(3-methylpropyl)vinyl group, a 1,3-dimethyl-1-butenyl group, a 1,3-dimethyl-2-butenyl group, a 1,3-dimethyl-3-butenyl group, a 2,3-dimethyl-1-butenyl group, a 2,3-dimethyl-2-butenyl group, a 2,3-dimethyl-3-butenyl group, a 3,3-dimethyl-1-butenyl group, a 2,2-dimethyl-3-butenyl group, a 1,1-dimethyl-2-butenyl group, a 1,1-dimethyl-3-butenyl group, a 1,1,2-trimethyl-2-propenyl group, a 1-ethyl-1-methyl-2-propenyl group, a 1-ethyl-2-methyl-1-propenyl group, a 1-ethyl-2-methyl-2-propenyl group, a 1-(1-methylethyl)-1-propenyl group, and a 1-(1-methylethyl)-2-propenyl group.

Any cycloalkenyl group may be used as long as the carbon atom at the node and a C═C double bond are included as an atom constituting a ring, the cycloalkenyl group may be condensed with cycloalkane, cycloalkene, an aromatic ring, or a hetero ring, and may form a spiro ring, and the number of carbon atoms is preferably 3, 4, 5, 6, 7, or 8. Examples of the cycloalkenyl group may include a 1-cyclopropen-1-yl group, a 2-cyclopropen-1-yl group, a 1-cyclobuten-1-yl group, a 2-cyclobuten-1-yl group, a 1-cyclopenten-1-yl group, a 2-cyclopenten-1-yl group, a 3-cyclopenten-1-yl group, a 1-cyclohexen-1-yl group, a 2-cyclohexen-1-yl group, a 3-cyclohexen-1-yl group, a 1-cyclohepten-1-yl group, a 2-cyclohepten-1-yl group, a 3-cyclohepten-1-yl group, a 4-cyclohepten-1-yl group, a 1-cycloocten-1-yl group, a 2-cycloocten-1-yl group, a 3-cycloocten-1-yl group, a 4-cycloocten-1-yl group, a 1,3-cyclopentadien-1-yl group, a 2,4-cyclopentadien-1-yl group, a 1,3-cyclohexadien-1-yl group, a 1,4-cyclohexadien-1-yl group, a 1,5-cyclohexadien-1-yl group, a 2,4-cyclohexadien-1-yl group, a 2,5-cyclohexadien-1-yl group, a 1,3-cycloheptadien-1-yl group, a 1,4-cycloheptadien-1-yl group, a 1,5-cycloheptadien-1-yl group, a 1,6-cycloheptadien-1-yl group, a 2,4-cycloheptadien-1-yl group, a 2,5-cycloheptadien-1-yl group, a 2,6-cycloheptadien-1-yl group, a 1,4-cycloheptadien-1-yl group, a 1,5-cycloheptadien-1-yl group, a 3,5-cycloheptadien-1-yl group, a 1,3-cyclooctadien-1-yl group, a 1,4-cyclooctadien-1-yl group, a 1,5-cyclooctadien-1-yl group, a 1,6-cyclooctadien-1-yl group, a 1,7-cyclooctadien-1-yl group, a 2,4-cyclooctadien-1-yl group, a 2,5-cyclooctadien-1-yl group, a 2,6-cyclooctadien-1-yl group, a 2,7-cyclooctadien-1-yl group, a 3,5-cyclooctadien-1-yl group, a 3,6-cyclooctadien-1-yl group, a 1,3,5-cycloheptatrien-1-yl group, a 1,3,6-cycloheptatrien-1-yl group, a 1,4,6-cycloheptatrien-1-yl group, a 2,4,6-cycloheptatrien-1-yl group, a 1,3,5-cyclooctatrien-1-yl group, a 1,3,6-cyclooctatrien-1-yl group, a 1,3,7-cyclooctatrien-1-yl group, a 1,4,6-cyclooctatrien-1-yl group, a 1,4,7-cyclooctatrien-1-yl group, a 1,5,7-cyclooctatrien-1-yl group, a 2,4,6-cyclooctatrien-1-yl group, a 2,4,7-cyclooctatrien-1-yl group, and a cyclooctatetraen-1-yl group.

As the alkynyl group, any of a linear, branched chain, or cyclic alkynyl group may be used, and the number of carbon atoms is preferably 2, 3, 4, 5, or 6. Examples of the alkynyl group may include an ethynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-2-butynyl group, a 1-methyl-3-butynyl group, a 2-methyl-3-butynyl group, a 3-methyl-1-butynyl group, a 1-ethyl-2-propynyl group, a 1,1-dimethyl-2-propynyl group, a 1-hexynyl group, a 2-hexynyl group, a 3-hexynyl group, a 4-hexynyl group, a 1-methyl-2-pentynyl group, a 1-methyl-3-pentynyl group, a 1-methyl-4-pentynyl group, a 2-methyl-3-pentynyl group, a 2-methyl-4-pentynyl group, a 3-methyl-1-pentynyl group, a 3-methyl-4-pentynyl group, a 4-methyl-1-pentynyl group, a 4-methyl-2-pentynyl group, a 1-butyl-2-propynyl group, a 1-ethyl-2-butynyl group, a 1-ethyl-3-butynyl group, a 2-ethyl-3-butynyl group, a 1,1-dimethyl-2-butynyl group, a 1,1-dimethyl-3-butynyl group, a 1,2-dimethyl-3-butynyl group, a 2,2-dimethyl-3-butynyl group, a 3,3-dimethyl-1-butynyl group, a 1-ethyl-1-methyl-2-propynyl group, a 1-(2-methylethyl)-2-propynyl group, a 2-cyclohexin-1-yl group, and a 3-cyclohexin-1-yl group.

As the aromatic group, a monocyclic or polycyclic aromatic group may be used, the aromatic group may be condensed with cycloalkane, cycloalkene, an aromatic ring, or a hetero ring, and the number of carbon atoms is preferably 6, 7, 8, 9, 10, 11, 12, 13, or 14. Examples of the aromatic group may include a phenyl group, a naphthyl group, and an anthracenyl group.

The heterocyclic group contains at least one or more of heteroatoms such as a nitrogen atom, an oxygen atom, or a sulfur atom as a ring-constituting atom, those atoms may be condensed with cycloalkane, cycloalkene, an aromatic ring, or a hetero ring or form a spiro ring, and the size of the ring is preferably a 3-, 4-, 5-, 6-, 7- or 8-membered ring. Examples of the heterocyclic group may include an aziridinyl group, an azetidinyl group, a diazetidinyl group, a pyrrolidinyl group, a piperidino group, a homopiperidino group, a pyrazolidinyl group, an imidazolidinyl group, a triazolidinyl group, a tetrazolidinyl group, an oxazolidinyl group, an isooxazolidinyl group, a thiazolidinyl group, an isothiazolidinyl group, an oxadiazolidinyl group, a thiadiazolidinyl group, a piperazinyl group, a homopiperazinyl group, a triazepanyl group, a morpholino group, a thiomorpholino group, a quinuclidinyl group, a tropanyl group, a pyrrolinyl group, a pyrazolinyl group, an imidazolinyl group, an oxazolinyl group, a thiazolinyl group, an isooxazolinyl group, an isothiazolinyl group, a pyrrolyl group, an imidazolyl group, a pyrazolyl group, an oxazolyl group, a dihydrooxazolyl group, a tetrahydrooxazolyl group, an isooxazolyl group, a dihydroisooxazolyl group, a tetrahydroisooxazolyl group, a thiazolyl group, a dihydrothiazolyl group, a tetrahydrothiazolyl group, an isothiazolyl group, a dihydroisothiazolyl group, a tetrahydroisothiazolyl group, a triazolinyl group, a triazolyl group, an oxodiazolyl group, a dihydrooxodiazolyl group, a tetrahydrooxodiazolyl group, a thiadiazolyl group, a dihydrothiadiazolyl group, a tetrahydrothiadiazolyl group, a tetrazolinyl group, a tetrazolyl group, a furazanyl group, a dihydrofurazanyl group, a tetrahydrofurazanyl group, a piperideinyl group, a triazinanyl group, a pyridyl group, a dihydropyridyl group, a tetrahydropyridyl group, a pyrazinyl group, a dihydropyrazinyl group, a tetrahydropyrazinyl group, a pyrimidinyl group, a dihydropyrimidinyl group, a tetrahydropyrimidinyl group, a perhydropyrimidinyl group, a pyridazinyl group, a dihydropyridazinyl group, a tetrahydropyridazinyl group, a perhydropyridazinyl group, a triazinyl group, a dihydrotriazinyl group, a tetrahydrotriazinyl group, an oxazinyl group, a dihydrooxazinyl group, a tetrahydrooxazinyl group, an oxadiazinyl group, a dihydrooxadiazinyl group, a tetrahydrooxadiazinyl group, a thiazinyl group, a dihydrothiazinyl group, a tetrahydrothiazinyl group, a thiadiazinyl group, a dihydrothiadiazinyl group, a tetrahydrothiadiazinyl group, an azepinyl group, a dihydroazepinyl group, a tetrahydroazepinyl group, a perhydroazepinyl group, a diazepinyl group, a dihydrodiazepinyl group, a tetrahydrodiazepinyl group, a perhydrodiazepinyl group, an oxazepinyl group, a dihydrooxazepinyl group, a tetrahydrooxazepinyl group, a perhydrooxazepinyl group, an oxadiazepinyl group, a dihydrooxadiazepinyl group, a tetrahydrooxadiazepinyl group, a perhydrooxadiazepinyl group, a thiazepinyl group, a dihydrothiazepinyl group, a tetrahydrothiazepinyl group, a perhydrothiazepinyl group, a thiadiazepinyl group, a dihydrothiadiazepinyl group, a tetrahydrothiadiazepinyl group, a perhydrothiadiazepinyl group, a triazepinyl group, a dihydrotriazepinyl group, a tetrahydrotriazepinyl group, a perhydrotriazepinyl group, an azocinyl group, a dihydroazocinyl group, a tetrahydroazocinyl group, an oxohydroazocinyl group, a perhydroazocinyl group, a morphanylgroup, a benzazocinyl group, an azepindolyl group, an indolinyl group, an indoleninyl group, an isoindolinyl group, an isoindoleninyl group, an indolyl group, a perhydroindolyl group, an isoindolyl group, a perhydroisoindolyl group, an indolizinyl group, an indolizidinyl group, an imidazopyridyl group, an indazolyl group, a dihydroindazolyl group, a perhydroindazolyl group, a benzimidazolyl group, a dihydrobenzimidazolyl group, a perhydrobenzimidazolyl group, a benzoxazolyl group, a dihydrobenzoxazolyl group, a perhydrobenzoxazolyl group, a benzothiazolyl group, a dihydrobenzothiazolyl group, a perhydrobenzothiazolyl group, a benzoxadiazolyl group, a benzothiadiazolyl group, a benzotriazolyl group, a purinyl group, a quinolinyl group, a dihydroquinolinyl group, a tetrahydroquinolinyl group, a perhydroquinolinyl group, a quinolizinyl group, a dihydroquinolizinyl group, a tetrahydroquinolizinyl group, an isoquinolinyl group, a dihydroisoquinolinyl group, a tetrahydroisoquinolinyl group, a perhydroisoquinolinyl group, a cinnolinyl group, a dihydrocinnolinyl group, a tetrahydrocinnolinyl group, a perhydrocinnolinyl group, a quinazolinyl group, a dihydroquinazolinyl group, a tetrahydroquinazolinyl group, a perhydroquinazolinyl group, a phthalazinyl group, a dihydrophthalazinyl group, a tetrahydrophthalazinyl group, a perhydrophthalazinyl group, a quinoxalinyl group, a dihydroquinoxalinyl group, a tetrahydroquinoxalinyl group, a perhydroquinoxalinyl group, a naphthyridinyl group, a dihydronaphthyridinyl group, a tetrahydronaphthyridinyl group, a perhydronaphthyridinyl group, a pteridinyl group, a quinolizidinyl group, a dihydrobenzoxazinyl group, a dihydrobenzothiazinyl group, a benzazepinyl group, a dihydrobenzazepinyl group, a tetrahydrobenzazepinyl group, a benzodiazepinyl group, a dihydrobenzodiazepinyl group, a tetrahydrobenzodiazepinyl group, a benzoxazepinyl group, a dihydrobenzoxazepinyl group, a tetrahydrobenzoxazepinyl group, a benzothiazepinyl group, a dihydrobenzothiazepinyl group, a tctrahydrobenzothiazepinyl group, a benzoxadiazepinyl group, a benzothiadiazepinyl group, a benzazepinyl group, a pyridoazepinyl group, a carbazolyl group, a dihydrocarbazolyl group, a tetrahydrocarbazolyl group, a perhydrocarbazolyl group, a β-carbolinyl group, a dihydro β-carbolinyl group, a tetrahydro β-carbolinyl group, a perhydro β-carbolinyl group, an acridinyl group, a dihydroacridinyl group, a tetrahydroacridinyl group, a perhydroacridinyl group, a phenazinyl group, a dihydrophenazinyl group, a tetrahydrophenazinyl group, a pcrhydrophenazinyl group, a phenothiazinyl group, a dihydrohydrophenothiazinyl group, a tetrahydrophenothiazinyl group, a perhydrophenothiazinyl group, a phenoxazinyl group, a dihydrophenoxazinyl group, a tetrahydrophenoxazinyl group, a perhydrophenoxazinyl group, a phenarsazinyl group, a phenanthridinyl group, a dihydrophenanthridinyl group, a tetrahydrophenanthridinyl group, a perhydrophenanthridinyl group, a phenanthrolinyl group, a dihydrophenanthrolinyl group, a tetrahydrophenanthrolinyl group, a perhydrophenanthrolinyl group, a perimidinyl group, a dihydroperimidinyl group, a tetrahydroperimidinyl group, a perhydroperimidinyl group, a pterinyl group, a pyrrolylidinyl group, a morphinanyl group, a hasubananyl group, a furyl group, a dihydrofuryl group, a tetrahydrofuryl group, a pyranyl group, a dihydropyranyl group, a tetrahydropyranyl group, an oxepinyl group, a dihydrooxepinyl group, a tetrahydrooxepinyl group, a perhydrooxepinyl group, a thienyl group, a dihydrothienyl group, a tetrahydrothienyl group, a thiopyranyl group, a dihydrothiopyranyl group, a tetrahydrothiopyranyl group, a thiepinyl group, a dihydrothiepinyl group, a tetrahydrothiepinyl group, a perhydrothiepinyl group, a benzofuryl group, a dihydrobenzofuryl group, a tetrahydrobenzofuryl group, a perhydrobenzofuryl group, an isobenzofuryl group, a dihydroisobenzofuryl group, a tetrahydroisobenzofuryl group, a perhydroisobenzofuryl group, a benzothienyl group, a dihydrobenzothienyl group, a tetrahydrobenzothienyl group, a perhydrobenzothienyl group, an isobenzothienyl group, a dihydroisobenzothienyl group, a tetrahydroisobenzothienyl group, a perhydroisobenzothienyl group, a benzopyranyl group, a dihydrobenzopyranyl group, a perhydrobenzopyranyl group, a benzothiopyranyl group, a dihydrobenzothiopyranyl group, a perhydrobenzothiopyranyl group, a benzoxepinyl group, a dihydrobenzoxepinyl group, a tetrahydrobenzoxepinyl group, a perhydrobenzoxepinyl group, a benzothiepinyl group, a dihydrobenzothiepinyl group, a tetrahydrobenzothiepinyl group, a perhydrobenzothiepinyl group, a benzofuryl group, a dihydrodibenzofuryl group, a tetrahydrodibenzofuryl group, a perhydrodibenzofuryl group, a xanthenyl group, a dihydroxanthenyl group, a tetrahydroxanthenyl group, a perhydroxanthenyl group, a benzothienyl group, a dihydrodibenzothienyl group, a tetrahydrodibenzothienyl group, a perhydrodibenzothienyl group, a thioxantenyl group, a dihydrothioxantenyl group, a tetrahydrothioxantenyl group, a perhydrothioxantenyl group, a phenoxathiinyl group, a dihydrophenoxathiinyl group, a tetrahydrophenoxathiinyl group, a perhydrophenoxathiinyl group, a dibenzodioxinyl group, a dihydrodibenzodioxinyl group, a tetrahydrodibenzodioxinyl group, a perhydrodibenzodioxinyl group, a thianthrenyl group, a dihydrothianthrenyl group, a tetrahydrothianthrenyl group, a perhydrothianthrenyl group, an oxiranyl group, an oxetanyl group, a thiiranyl group, a thietanyl group, an oxathiinyl group, a dihydrooxathiinyl group, a tetrahydrooxathiinyl group, a benzoxathiinyl group, a dihydrobenzoxathiinyl group, a tetrahydrobenzoxathiinyl group, a perhydrobenzoxathiinyl group, a benzodioxepanyl group, a dioxolanyl group, a dioxanyl group, a dithiolanyl group, a dithianyl group, a dioxoindanyl group, a benzodioxanyl group, a chromanyl group, a benzodithiolanyl group, and a benzodithianyl group, and in the case of an unsaturated heterocyclic group, a heterocyclic group in which at least a part is hydrogenated is also included.

Further, any two or three groups of the substituents R¹, R², R³, R⁴, R⁵, and R⁶ each may be combined together to form a ring. Examples of the ring may include cyclopropane, cyclopropene, cyclobutane, cyclobutene, cyclopentane, cyclopentene, cyclopentadiene, cyclohexane, cyclohexene, cyclohexadiene, cycloheptane, cycloheptene, cycloheptadiene, cycloheptatriene, cyclooctane, cyclooctene, cyclooctadiene, cyclooctatriene, aziridine, azetidine, diazetidine, pyrrolidine, piperidine, homopiperidine, pyrazolidine, imidazolidine, triazolidine, tetrazolidine, oxazolidine, isooxazolidine, thiazolidine, isothiazolidine, oxazodiazolidine, thiadiazolidine, piperazine, homopiperazine, triazepane, morpholine, thiomorpholine, quinuclidine, tropane, pyrroline, pyrazoline, imidazoline, oxazoline, thiazoline, isooxazoline, isothiazoline, dihydrooxazole, tetrahydrooxazole, dihydroisooxazole, tetrahydroisooxazole, dihydrothiazole, tetrahydrothiazole, dihydroisothiazole, tetrahydroisothiazole, triazoline, dihydrooxadiazole, tetrahydrooxadiazole, dihydrothiadiazole, tetrahydrothiadiazole, dihydrofurazan, tetrahydrofurazan, piperidyne, triazinane, dihydropyridine, tetrahydropyridine, dihydropyrazine, tetrahydropyrazine, dihydropyrimidine, tetrahydropyrimidine, perhydropyrimidine, dihydropyridazine, tetrahydropyridazine, perhydropyridazine, oxazine, dihydro oxazine, tetrahydro oxazine, oxadiazine, dihydro oxadiazine, tetrahydro oxadiazine, thiazine, dihydrothiazine, tetrahydrothiazine, thiadiazine, dihydrothiadiazine, tetrahydrothiadiazine, dihydroazepine, tetrahydroazepine, perhydroazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphan, azepindole, indoline, indolenine, isoindoline, isoindolenine, perhydroindole, perhydroisoindole, perhydroisoindole, indolizidine, dihydroindazole, perhydroindazole, dihydrobenzimidazole, perhydrobenzimidazole, dihydrobenzoxazole, perhydrobenzoxazole, dihydrobenzothiazole, perhydrobenzothiazole, dihydroquinoline, tetrahydroquinoline, perhydroquinoline, quinolizine, dihydroquinolizine, tetrahydroquinolizine, dihydroisoquinoline, tetrahydroisoquinoline, perhydroisoquinoline, dihydrocinnoline, tetrahydrocinnoline, perhydrocinnoline, dihydroquinazoline, tetrahydroquinazoline, perhydroquinazoline, dihydrophthalazine, tetrahydrophthalazine, perhydrophthalazine, dihydroquinoxaline, tetrahydroquinoxaline, perhydroquinoxaline, dihydronaphthyridine, tetrahydronaphthyridine, perhydronaphthyridine, quinolizidine, dihydrobenzoxazine, dihydrobenzothiazine, dihydrobenzazepine, tetrahydrobenzazepine, perhydrobenzazepine, dihydrobenzodiazcpine, tetrahydrobenzodiazepine, perhydrobenzodiazepine, dihydrobenzoxazepine, tetrahydrobenzoxazepine, perhydrobenzoxazepine, dihydrobenzothiazepine, tetrahydrobenzothiazepine, perhydrobenzothiazepine, dihydrocarbazole, tetrahydrocarbazole, perhydrocarbazole, dihydro β-carboline, tetrahydro β-carboline, perhydro β-carboline, dihydroacridine, tetrahydroacridine, perhydroacridine, dihydrophenazine, tetrahydrophenazine, perhydrophenazine, dihydrohydrophenothiazine, tetrahydrophenothiazine, perhydrophenothiazine, dihydrophenoxazine, tetrahydrophenoxazine, perhydrophenoxazine, dihydrophenanthridine, tetrahydrophenanthridine, perhydrophenanthridine, dihydrophenanthroline, tetrahydrophenanthroline, perhydrophenanthroline, dihydroperimidine, tetrahydroperimidine, perhydroperimidine, pyrrolizidine, morphinan, hasubanan, dihydrofuran, tetrahydrofuran, pyran, dihydropyran, tetrahydropyran, dihydrooxepin, tetrahydrooxepin, perhydrooxepin, dihydrothiophene, tetrahydrothiophene, thiopyran, dihydrothiopyran, tetrahydrothiopyran, dihydrothiepine, tetrahydrothiepine, perhydrothiepine, dihydrobenzofuran, tetrahydrobenzofuran, perhydrobenzofuran, dihydroisobenzofuran, tetrahydroisobenzofuran, perhydroisobenzofuran, dihydrobenzothiophene, tetrahydrobenzothiophene, perhydrobenzothiophene, dihydroisobenzothiophene, tetrahydrobenzothiophene, perhydrobenzothiophene, benzopyran, dihydrobenzopyran, perhydrobenzopyran, benzothiopyran, dihydrobenzothiopyran, perhydrobenzothiopyran, dihydrobenzoxepin, tetrahydrobenzoxepin, perhydrobenzoxepin, dihydrobenzothiepine, tetrahydrobenzothiepine, perhydrobenzothiepine, dihydrodibenzofuran, tetrahydrodibenzofuran, perhydrodibenzofuran, xanthene, dihydroxanthene, tetrahydroxanthene, perhydroxanthene, dihydrodibenzothiophene, tetrahydrodibenzothiophene, perhydrodibenzothiophene, thioxanthene, dihydrothioxanthene, tetrahydrothioxanthene, perhydrothioxanthene, dihydrophenoxathiin, tetrahydrophenoxathiin, perhydrophenoxathiin, dihydrodibenzodioxin, tetrahydrodibenzodioxin, perhydrodibenzodioxin, dihydrothianthrene, tetrahydrothianthrene, perhydrothianthrene, oxirane, oxetane, thiirane, thietane, dihydrooxathiin, tetrahydrooxathiin, dihydrobenzoxathiin, tetrahydrobenzoxathiin, perhydrobenzoxathiin, benzodioxepane, dioxolan, dioxane, dithiolane, dithiane, dioxoindane, benzodioxane, chromane, benzodithiolane, and benzodithiane, and in the case of an unsaturated ring, a ring in which at least a part is hydrogenated is also included. Further, in the case of forming a ring, it is preferable that any two substituents of R³ to R⁶ form a ring.

Further, examples of the substituent which the alkyl group, the cycloalkyl group, the alkenyl group, the cycloalkenyl group, the alkynyl group, the aromatic group, and the heterocyclic group may have include groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group. Herein, Rx, Ry, and Rz each independently represent a hydrogen atom, an alkyl group, a cycloalkyl group, an alkenyl group, a cycloalkenyl group, an alkynyl group, an aromatic hydrocarbon group, or a heterocyclic group. Further, Rx, Ry, and Rz may be bonded to each other to form a saturated or unsaturated hetero ring, and this ring can also form a condensed ring or a spiro ring with an aliphatic ring or a hetero ring and can also form a condensed ring with an aromatic ring.

Incidentally, Rx, Ry and Rz excluding the case of a hydrogen atom and the alkyl group, the cycloalkyl group, the alkenyl group, the cycloalkenyl group, the alkynyl group, the aromatic group, and the heterocyclic group as the substituent which are described herein include the same groups as the groups represented by R¹, R², R³, R⁴, R⁵, and R⁶. Further, the alkyl group of the alkoxy group and the alkylthio group as substituents has the same definition as the definition of the alkyl group in R¹, R², R³, R⁴, R⁵, and R⁶ described above, and the aryl group of the aryloxy group and the arylthio group has the same definition as the definition of the aromatic group in R¹, R², R³, R⁴, R⁵, and R⁶ described above.

Further, examples of a guanidino group, an acyl group, a substituted sulfonyl group, a heterocyclyloxy group, a heterocyclyl thio group, a carbamoyl group, a ureido group, an amide group, a sulfamoyl group, an acyloxy group, a sulfonamide group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, and an alkoxysulfonyl group as substituents are as follows.

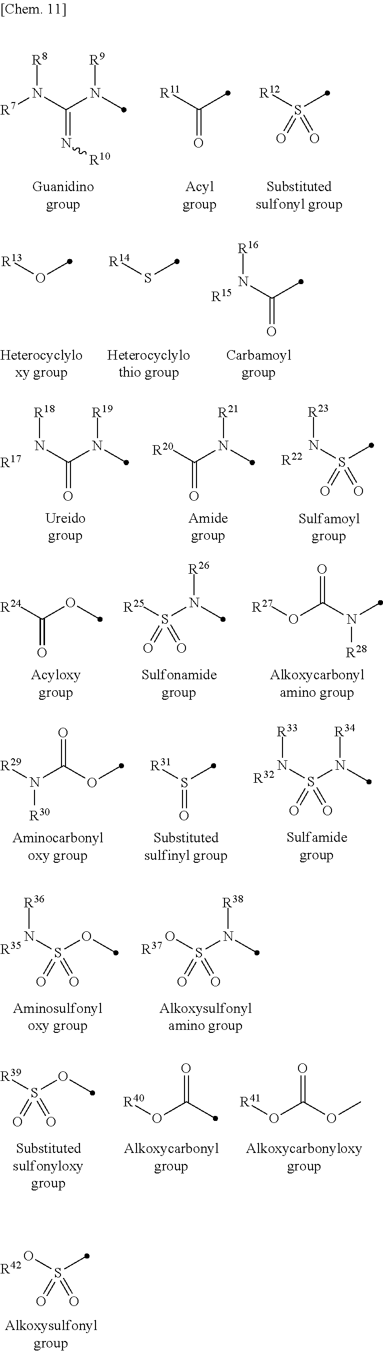

(In the exemplary groups, R⁷ to R¹², R¹⁵ to R²⁴, R²⁶, R²⁸ to R³⁶ and R³⁸ to R³⁹ represent a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R²⁵, R²⁷, R³⁷ and R⁴⁰ to R⁴² represent a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R¹³ and R¹⁴ represent a substituted or unsubstituted heterocyclic group. Further, examples of substituents of those substituted alkyl group, substituted cycloalkyl group, substituted alkenyl group, substituted cycloalkenyl group, substituted alkynyl group, substituted aromatic group, and substituted heterocyclic group include the same substituents as substituents of those groups in R¹, R², R³, R⁴, R⁵, and R⁶ described above.)

The groups represented by R¹ to R⁶ each independently is a hydrogen atom or an alkyl group having carbon number of 1 to 6, or two of R³ to R⁶ are coupled to form a cycloalkyl group having carbon number of 3 to 8 is preferable in terms of ease of availability of a raw material. Particularly, it is preferable that both of R¹ and R² represent a hydrogen atom or one of R¹ and R² represents a methyl group.

In the tertiary amine compound or imine compound-polymer conjugate represented by Formula (I) or (II) of the present invention and the amine form that is an important intermediate represented by Formula (III) or (IX), D⁺ is a structure in which the tertiary amine compound or imine compound D forms a quaternary ammonium salt or an iminium salt, and D specifically represents a compound represented by the following Formula (X).

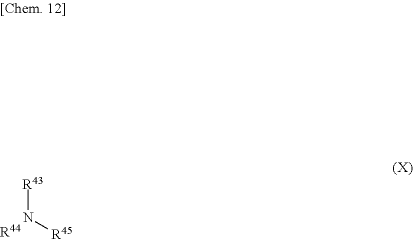

R⁴³, R⁴⁴, and R⁴⁵ each independently represent a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group, an R⁴⁶O— group, an R⁴⁷S— group, or an R⁴⁸(R⁴⁹)N— group (herein, R⁴⁶, R⁴⁷, R⁴⁸, and R⁴⁹ each independently represent a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, a substituted or unsubstituted heterocyclic group, or an Rx(Ry)N group), two of R⁴³, R⁴⁴, and R⁴⁵ may be combined together to form a double bond so as to form an imino group or azo group with N at the center, or at least two of R⁴³, R⁴⁴, and R⁴⁵ may be bonded to each other to form a saturated or unsaturated hetero ring, the ring can also form a condensed ring or a spiro ring with an aliphatic ring or a hetero ring, and a condensed ring can be formed with an aromatic ring. The alkyl group, the cycloalkyl group, the alkenyl group, the cycloalkenyl group, the alkynyl group, the aromatic group, or the heterocyclic group described herein has the same meaning as definition in R¹, R², R³, R⁴, R⁵, and R⁶ described above. Further, Rx and Ry described herein have the same meaning as definitions of Rx and Ry in the Rx(Ry)N group that is the substituent of R¹, R², R³, R⁴, R⁵, and R⁶ described above.

Examples of the saturated or unsaturated hetero ring formed by R⁴³, R⁴⁴, and R⁴⁵ being bonded to each other may include aziridine, azetidine, diazetidine, pyrrolidine, piperidine, homopiperidine, pyrazolidine, imidazolidine, triazolidine, tetrazolidine, oxazolidine, isooxazolidine, thiazolidine, isothiazolidine, oxadiazolidine, thiadiazolidine, piperazine, homopiperazine, triazepane, morpholine, thiomorpholine, quinuclidine, tropane, pyrroline, pyrazoline, imidazoline, oxazoline, thiazoline, isooxazoline, isothiazoline, pyrrol, imidazole, pyrazole, oxazole, dihydrooxazole, tetrahydrooxazole, isooxazole, dihydroisooxazole, tetrahydroisooxazole, thiazole, dihydrothiazole, tetrahydrothiazole, isothiazole, dihydroisothiazole, tetrahydroisothiazole, triazoline, triazole, oxodiazole, dihydrooxodiazole, tetrahydrooxodiazole, thiadiazole, dihydrothiadiazole, tetrahydrothiadiazole, tetrazoline, tetrazole, furazan, dihydrofurazan, tetrahydrofurazan, piperidyne, triazinane, pyridine, dihydropyridine, tetrahydropyridine, pyrazine, dihydropyrazine, tetrahydropyrazine, pyrimidine, dihydropyrimidine, tetrahydropyrimidine, perhydropyrimidine, pyridazine, dihydropyridazine, tetrahydropyridazine, perhydropyridazine, triazine, dihydrotriazine, tetrahydrotriazine, oxazine, dihydro oxazine, tetrahydro oxazine, oxadiazine, dihydro oxadiazine, tetrahydro oxadiazine, thiazine, dihydrothiazine, tetrahydrothiazine, thiadiazine, dihydrothiadiazine, tetrahydrothiadiazine, azepine, dihydroazepine, tetrahydroazepine, perhydroazepine, diazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphan, benzazocine, azepindole, indoline, indolenine, isoindoline, isoindolenine, indole, perhydroindole, isoindole, perhydroisoindole, indolizine, indolizidine, imidazopyridine, indazole, dihydroindazole, perhydroindazole, benzimidazole, dihydrobenzimidazole, perhydrobenzimidazole, benzoxazole, dihydrobenzoxazole, perhydrobenzoxazole, benzothiazole, dihydrobenzothiazole, perhydrobenzothiazole, benzoxadiazole, benzothiadiazole, benzotriazole, purine, quinoline, dihydroquinoline, tetrahydroquinoline, perhydroquinoline, quinolizine, dihydroquinolizine, tetrahydroquinolizine, isoquinoline, dihydroisoquinoline, tetrahydroisoquinoline, perhydroisoquinoline, cinnoline, dihydrocinnoline, tetrahydrocinnoline, perhydrocinnoline, quinazoline, dihydroquinazoline, tetrahydroquinazoline, perhydroquinazoline, phthalazine, dihydrophthalazine, tetrahydrophthalazine, perhydrophthalazine, quinoxaline, dihydroquinoxaline, tetrahydroquinoxaline, perhydroquinoxaline, naphthyridine, dihydronaphthyridine, tetrahydronaphthyridine, perhydronaphthyridine, pteridine, quinolizidine, dihydrobenzoxazine, dihydrobenzothiazine, benzazepine, dihydrobenzazepine, tetrahydrobenzazepine, benzodiazepine, dihydrobenzodiazepine, tetrahydrobenzodiazepine, benzoxazepine, dihydrobenzoxazepine, tetrahydrobenzoxazepine, benzothiazepine, dihydrobenzothiazepine, tetrahydrobenzothiazepine, benzoxadiazepine, benzothiazeazepine, benzazepine, pyridoazepine, carbazole, dihydrocarbazole, tetrahydrocarbazole, perhydrocarbazole, β-carboline, dihydro β-carboline, tetrahydro β-carboline, perhydro β-carboline, acridine, dihydroacridine, tetrahydroacridine, perhydroacridine, phenazine, dihydrophenazine, tetrahydrophenazine, perhydrophenazine, phenothiazine, dihydrohydrophenothiazine, tetrahydrophenothiazine, perhydrophenothiazine, phenoxazine, dihydrophenoxazine, tetrahydrophenoxazine, perhydrophenoxazine, phenarsazine, phenanthridine, dihydrophenanthridine, tetrahydrophenanthridine, perhydrophenanthridine, phenanthroline, dihydrophenanthroline, tetrahydrophenanthroline, perhydrophenanthroline, perimidine, dihydroperimidine, tetrahydroperimidine, perhydroperimidine, pterin, pyrrolizidine, morphinan, hasubanan, and pyridinomorpholine, and in the case of an unsaturated hetero ring, a hetero ring in which at least a part is hydrogenated is also included. Further, a structure in which two or more of those structures are bonded to each other directly or via an alkylene group can also be employed, and the heterocyclic group has the same definition as that of the heterocyclic group represented by R¹, R², R³, R⁴, R⁵, and R⁶ described above and can have a substituent. A specific structure is not particularly limited as long as it has a structure of a tertiary amine or imine compound and can form an ammonium salt or an iminium salt, but it is preferable that a structure having a 4-cyanoguanidinopyridine or 3-carbamoylpyridine skeleton is not employed.

Examples of a substituent which the alkyl group, the cycloalkyl group, the alkenyl group, the cycloalkenyl group, the alkynyl group, the aromatic group, the heterocyclic group, the R⁴⁶O— group, the R⁴⁷S— group, the R⁴⁸(R⁴⁹)N— group and the saturated or unsaturated hetero ring formed by R⁴³, R⁴⁴, and R⁴⁵ being bonded to each other may have include the same substituents of those groups in R¹, R², R³, R⁴, R⁵, and R⁶ described above.

Further, D⁺ is a structure in which the tertiary amine compound or imine compound D forms a quaternary ammonium salt or an iminium salt, and the tertiary amine compound or imine compound D is preferably a compound having bioactivity. Examples of the compound having bioactivity may include a medical drug, a quasi-drug, a medical instrument, an in-vitro diagnostic medical drug, a tissue-engineered medical product, a medical drug for animals, an agricultural chemical, and a supplement. If the released tertiary amine compound or imine compound D has bioactivity and can form a quaternary ammonium salt or iminium salt structure, the structure of the compound is not limited, and a known compound which can be used as a compound having bioactivity can be used as the tertiary amine compound or imine compound D.

In the amine form represented by Formula (III) or (IX), X⁻ represents a counter anion of the quaternary ammonium salt or iminium salt in D⁺, and examples thereof include a halide ion such as a chloride ion, a bromide ion, and an iodide ion; an inorganic acid anion such as a sulfate ion and a nitric acid ion; and an organic acid anion such as a trifluoroacetic acid ion, a methanesulfonic acid ion, a toluenetoluenesulfonate ion, and a trifluoromethanesulfonate ion. Further, the amine form represented by Formula (III) or (IX) may form a salt with an inorganic acid or an organic acid, examples of the inorganic acid include hydrochloric acid, sulfuric acid, and nitric acid, and examples of the organic acid include trifluoroacetic acid, methanesulfonic acid, toluenesulfonic acid, benzenesulfonic acid, and trifluoromethanesulfonic acid. Regarding a salt formed with an inorganic acid or an organic acid, preferably, an amino group existing at the molecule terminal of the amine form represented by Formula (III) or (IX) forms a salt with an inorganic acid or an organic acid.

A structure derived from the polymer having a carboxy group is a structure represented by Formula (IV):

Poly-CO₂H (IV)

that is a polymer having one or a plurality of carboxy groups in the molecule. Hereinafter, the moiety of Poly is also referred to as “polymer residue having a carboxy group.” The polymer may be a naturally derived polymer or artificially synthesized polymer. The artificially synthesized polymer may be, for example, a polymer obtained by polymerizing monomers having a carboxy group or one in which a carboxy group is introduced into a polymer originally having no carboxy group by chemical modification. Further, in the case of having a plurality of carboxy groups, the amine form represented by Formula (III) or (IX) may be formed by condensing the plurality of carboxy groups, the carboxy group remaining without being condensed with the amine form represented by Formula (III) or (IX) may exist as a free carboxy group, a salt may be formed using a metal such as lithium, sodium, potassium, magnesium, or calcium or an organic base such as triethylamine, tributylamine, or pyridine, or a salt may be formed using tetrabutylammonium hydroxide. Examples of the polymer having a carboxy group include synthetic polymers such as polyacrylic acid, polymethacrylic acid, polymaleic acid, polylactic acid (PLA), polyglycolic acid (PGA), lactic acid-glycolic acid copolymer (PLGA), polycaprolactone, polycarboxyisopropylacrylamide, polyethylene terephthalate, polybutylene terephthalate, and carboxy group-modified polyethylene glycol; natural polysaccharides such as alginic acid, hyaluronic acid, heparin, chondroitin, chondroitin sulfate (A, B, C, D, and E), keratan sulfate, heparan sulfate, dermatan sulfate, pectin (homogalacturonan and rhamnogalacturonan), xanthane gum, xylan, and sacran; semisynthetic polymers such as carboxymethyl cellulose, carboxymethyl chitin, carboxymethyl chitosan, carboxymethyl dextran, carboxymethyl amylose, succinyl chitosan, and polyethylene glycol into which a carboxy group is inserted; polyamino acids such as polyasparaginic acid, polyglutamic acid, and protein; and nucleic acids such as deoxyribonucleic acid into which a carboxy group is introduced. Examples of a water-soluble polymer having a carboxy group include synthetic polymers such as polyacrylic acid, polymethacrylic acid, polymaleic acid, polycarboxyisopropylacrylamide, and carboxy group-modified polyethylene glycol; natural polysaccharides such as alginic acid, hyaluronic acid, heparin, chondroitin, chondroitin sulfate (A, B, C, D, and E), keratan sulfate, heparan sulfate, dermatan sulfate, pectin(homogalacturonan and rhamnogalacturonan), xanthane gum, xylan, and sacran; semisynthetic polymers such as carboxymethyl cellulose, carboxymethyl chitin, carboxymethyl chitosan, carboxymethyl dextran, carboxymethyl amylose, succinyl chitosan, and polyethylene glycol into which a carboxy group is inserted; polyamino acids such as polyasparaginic acid, polyglutamic acid, and protein; and nucleic acids such as deoxyribonucleic acid into which a carboxy group is introduced. Those polymers having a carboxy group may be further modified or cross-linked by various methods in some cases.

Poly, the polymer residue having a carboxy group, means a partial structure of the polymer having a carboxy group represented by Formula (IV) excluding a carboxy group moiety used in condensation with an amine form represented by Formula (III) or (IX). As the polymer residue Poly, a water-soluble polymer residue, a polysaccharide residue, a glycosaminoglycan residue, a chondroitin residue, a chondroitin sulfate residue, and a hyaluronic acid residue can be exemplified as a preferable aspect. These residues each mean a partial structure of water-soluble polymer, polysaccharide, glycosaminoglycan, chondroitin, chondroitin sulfate, and hyaluronic acid excluding a carboxy group condensed with a compound (III) or (IX).

A production example of the tertiary amine compound or imine compound-polymer conjugate represented by Formula (I) of the present invention is as follows:

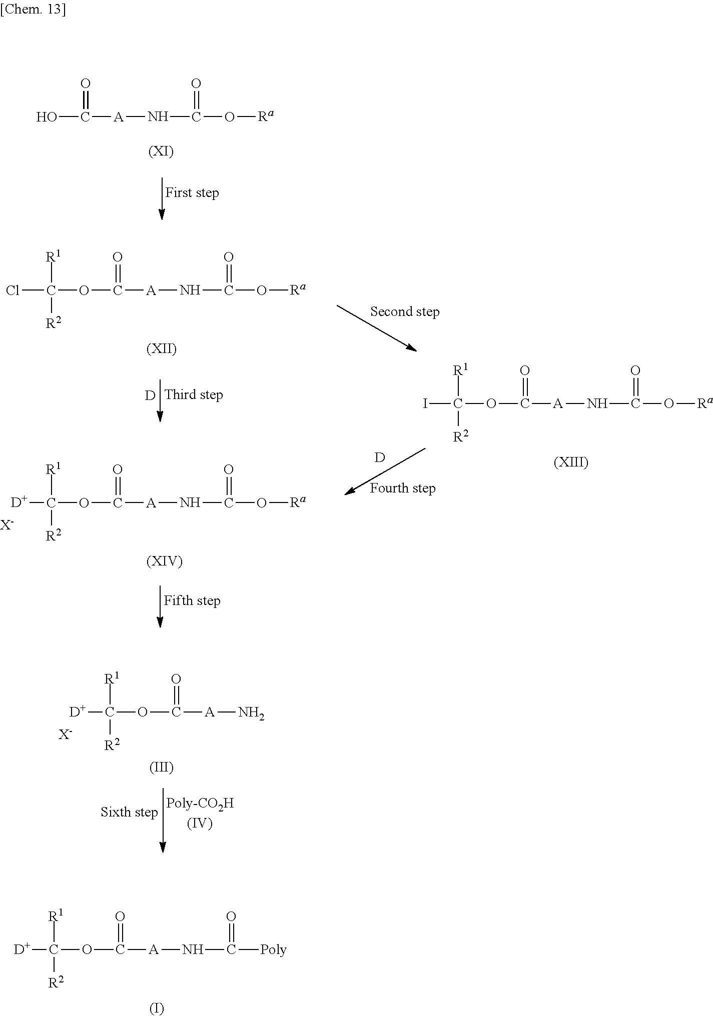

(in the formula, R^{a} represents a benzyl group or a t-butyl group, and R¹ to R⁶, D⁺, X⁻, A, l, m, n, and Poly are as defined above.)

### First Step

This step is to produce the chloromethyl ester form represented by Formula (XII) from the protection amino acid represented by Formula (XI). This step can be performed by chloroalkyl chlorosulfonate reacting in the presence of a base. As the base, for example, sodium hydrogen carbonate, sodium carbonate, potassium bicarbonate, potassium carbonate, sodium hydroxide, potassium hydroxide, lithium hydroxide, and the like can be used. As the chloroalkyl sulfonyl chloride, for example, chloromethylchlorosulfonate, 1-chloroethylchlorosulfonate, or the like can be used.

Upon performing this step, this step is preferably performed in a solvent, for example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used, and if necessary, a mixed solvent of an organic solvent and water can be used. Further, if necessary, a phase transfer catalyst can be used, and as the phase transfer catalyst, for example, tetrabutylammonium hydrogen sulfate, tetrabutylammonium chloride, tetrabutylammonium bromide, tetrabutylammonium iodide, or the like can be used. As the reaction temperature, the step can be advanced generally in a range of −30° C. to 200° C. and preferably in a range of −15° C. to 80° C.

### Second Step

This step is to produce the iodomethyl ester form represented by Formula (XIII) by iodizing the chloromethyl ester form represented by Formula (XII). As an iodizing agent to be used in this step, for example, sodium iodide, potassium iodide, or the like can be used.

Upon performing this step, this step is preferably performed in a solvent, and for example, an organic solvent such as ethyl acetate, acetone, benzene, toluene, xylene, dimethylformamide, acetonitrile, tetrahydrofuran, dioxane, diethyl ether, diisopropyl ether, or dimethoxyethane can be used. As the reaction temperature, the step can be advanced generally in a range of 0° C. to 200° C. and preferably in a range of 10° C. to 150° C.

### Third Step

This step is to produce the quaternary ammonium salt or iminium salt represented by Formula (XIV) by reacting the chloromethyl ester form represented by Formula (XII) with the tertiary amine compound or imine compound represented by D.

Upon performing this step, this step can be performed in an organic solvent or in the absence of a solvent. As the organic solvent, for example, methylene chloride, chloroform, dichloroethane, ethyl acetate, acetone, benzene, toluene, xylene, dimethylformamide, acetonitrile, tetrahydrofuran, dioxane, diethyl ether, diisopropyl ether, dimethoxyethane, methanol, ethanol, 1-propanol, 2-propanol, or the like can be used. As the reaction temperature, the step can be advanced generally in a range of 0° C. to 200° C. and preferably in a range of 20° C. to 150° C.

### Fourth Step

This step is to produce the quaternary ammonium salt or iminium salt represented by Formula (XIV) by reacting the iodomethyl ester represented by Formula (XIII) with the tertiary amine compound or imine compound represented by D.

Upon performing this step, this step can be performed in an organic solvent or in the absence of a solvent. As the organic solvent, for example, methylene chloride, chloroform, dichloroethane, ethyl acetate, acetone, benzene, toluene, xylene, dimethylformamide, acetonitrile, tetrahydrofuran, dioxane, diethyl ether, diisopropyl ether, dimethoxyethane, methanol, ethanol, 1-propanol, 2-propanol, or the like can be used. As the reaction temperature, the step can be advanced generally in a range of 0° C. to 200° C. and preferably in a range of 10° C. to 100° C.

Further, in this step, the reaction can also be advanced while the iodomethyl ester form represented by Formula (XIII) is not isolated but is generated in the reaction system. That is, the chloromethyl ester form represented by Formula (XII) can also be reacted with the tertiary amine compound or imine compound represented by D in the presence of an iodizing agent. In this case, as the iodizing agent, for example, sodium iodide, potassium iodide, or the like can be used, and as the solvent, acetone, acetonitrile, dioxane, tetrahydrofuran, toluene, ethyl acetate, dimethylformamide, dimethoxyethane, or the like can be used. As the reaction temperature, the step can be advanced generally in a range of 0° C. to 200° C. and preferably in a range of 10° C. to 150° C.

### Fifth Step

This step is to produce the amine form represented by Formula (II) by deprotecting the quaternary ammonium salt or iminium salt represented by Formula (XIV).

In this step, in a case where R^{a} represents a benzyl group, the quaternary ammonium salt or iminium salt is deprotected by catalytic hydrogen addition so that the amine form represented by Formula (III) can be produced. For example, a platinum catalyst such as platinum oxide or platinum carbon, a palladium catalyst such as palladium carbon, palladium black, or palladium oxide, or a nickel catalyst such as Raney nickel can be used. Upon performing this step, this step is preferably performed in a solvent, and for example, methanol, ethanol, isopropyl alcohol, tetrahydrofuran, dimethylformamide, dioxane, water, or the like can be used. As the reaction temperature, the step can be advanced generally in a range of −50° C. to 200° C. and preferably in a range of 10° C. to 100° C.

In this step, in a case where R^{a} represents a t-butyl group, the amine form represented by Formula (III) can be produced by deprotection using an acid. As the acid, for example, hydrogen chloride, hydrochloric acid, sulfuric acid, nitric acid, methanesulfonic acid, benzenesulfonic acid, p-toluenesulfonic acid, trifluoromethanesulfonic acid, trifluoroacetic acid, or the like can be used. The amine form represented by Formula (III) which is obtained in this step is produced by forming salts with those acids. In this step, the reaction can be advanced in the absence of a solvent or in a solvent, and as the solvent, for example, ethyl acetate, dioxane, methanol, ethanol, 1-propanol, 2-propanol, water, or the like can be used. As the reaction temperature, the step can be advanced generally in a range of −50° C. to 200° C. and preferably in a range of 0° C. to 120° C.

### Sixth Step

This step is to produce the tertiary amine compound or imine compound-polymer conjugate represented by Formula (I) by condensing the amine form represented by Formula (III) with the polymer having a carboxy group represented by Formula (IV). As the condensing agent to be used herein, for example, 1-ethyl-3-(3-dimethylaminopropyl)carbodiimide hydrochloride (EDC or WSC), 4-(4,6-dimethoxy-1,3,5-triazin-2-yl)-4-methylmorpholinium chloride (DMT-MM), tetramethylfluoroformamidinium hexafluorophosphate (TFFH), bis(tetramethylene)fluoroformamidinium hexafluorophosphate (BTFFH), or the like can be used. Further, in a case where the carboxy group of the polymer having a carboxy group is derivatized into an active ester such as N-hydroxysuccinimide ester or p-nitrophenyl ester, it is not necessary to add a condensing agent, and condensation can also be performed by only mixing with the amine form represented by Formula (III), or if necessary, adding a base.

This step is preferably performed in a solvent, and for example, water or an organic solvent such as methylene chloride, chloroform, dichloroethane, toluene, ethyl acetate, acetone, dimethylformamide, formamide, N-methylpyrrolidone, acetonitrile, tetrahydrofuran, dioxane, diethyl ether, dimethoxyethane, dimethyl sulfoxide, methanol, ethanol, 1-propanol, 2-propanol, or ethylene glycol can be used. Further, those organic solvents and water are mixed at an arbitrary ratio and can also be used as a mixed solvent.

This step is, more specifically, a step for producing a conjugate represented by the following Formula (II), the step including a step of condensing a compound represented by the following Formula (IX) and a polymer having a carboxy group represented by the following Formula (IV):

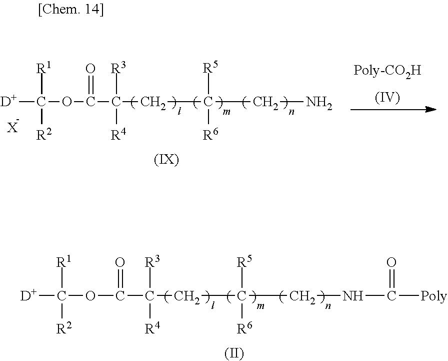

[in Formulae (II), (IV), and (IX), D⁺, R¹, R², R³, R⁴, R⁵, R⁶, l, n, m, and Poly are as defined above, X⁻ represents a counter anion of D⁺, and the compound represented by Formula (IX) may form a salt with an inorganic acid or an organic acid].

A further another aspect of the present invention is a linker represented by the following Formula (V) for bonding a tertiary amine compound containing a nitrogen atom capable of forming a quaternary ammonium salt or an imine compound capable of forming an iminium salt to a polymer having a carboxy group:

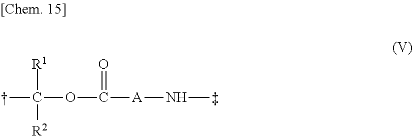

(herein, R¹, R², and A are as defined above. Symbol † represents a node with a nitrogen atom forming a quaternary ammonium salt or an iminium salt, and symbol ‡ means a node with a moiety of the carboxy group of the polymer having a carboxy group excluding a hydroxyl group.)

The conjugate of the present invention can be obtained by the method exemplified in the above-described steps 1 to 6 using the linker represented by Formula (V). Therefore, a further another aspect of the present invention is a method for producing the compound represented by Formula (I) using the linker represented by Formula (V), the method including a step of bonding a tertiary amine compound containing a nitrogen atom capable of forming a quaternary ammonium salt or an imine compound capable of forming an iminium salt to a polymer having a carboxy group via the linker. The linker is more specifically represented by the following Formula (XV):

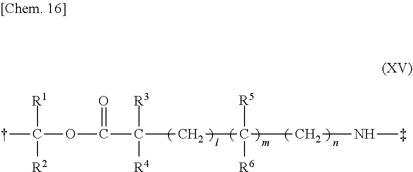

(herein, R¹, R², R³, R⁴, R⁵, R⁶, l, m, and n in the above (XV) are as defined above, symbol † represents a node with a nitrogen atom forming a quaternary ammonium salt or an iminium salt, and symbol ‡ means a node with a moiety of the carboxy group of the polymer having a carboxy group excluding a hydroxyl group.)

The tertiary amine compound or imine compound-polymer conjugate of the present invention is a conjugate whose release rate can be controlled and is expected to be used in medical drugs and the like, as clearly shown from Test Example described later.

### EXAMPLES

Hereinafter, the present invention will be described in more detail by means of Reference Examples and Examples; however, the present invention is not limited to those examples without departing from the scope thereof.

Further, synthesis examples of the chloromethyl ester form represented by Formula (XII), the iodomethyl ester form represented by Formula (XIII), and the quaternary ammonium salt or iminium salt represented by Formula (XIV) which are intermediates for producing the tertiary amine compound or imine compound-polymer conjugate of the present invention are presented as Reference Examples.

### Reference Example 1

### N-[(1,1-dimethylethoxy)carbonyl]-glycine Chloromethyl Ester

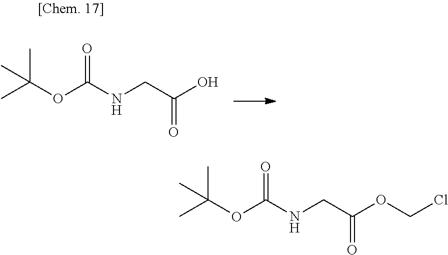

Under cooling on ice, a methylene chloride solution of 1.98 g (12 mmol) of chloromethyl chlorosulfonate was added dropwise to a water 20 ml-methylene chloride 20 ml mixed solution of 1.75 g (10 mmol) of N-[(1,1-dimethylethoxy)carbonyl]-glycine, 340 mg (1 mmol) of tetrabutylammonium hydrogen sulfate, and 3.36 g (40 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (10% to 20% ethyl acetate/hexane) to obtain 1.85 g (83%) of the title compound.

¹H-NMR (CDCl₃, δ):1.46 (9H, S), 4.00 (2H, d, J=6 Hz), 4.98 (1H, br-s), 5.75 (2H, s)

### Reference Example 2

### N-[(1,1-dimethylethoxy)carbonyl]-glycine Iodomethyl Ester

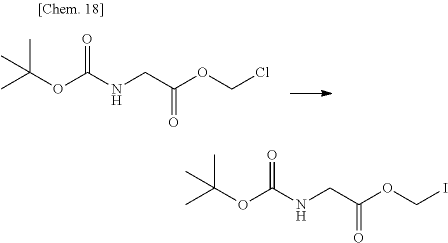

A suspension of 1.85 g (8.3 mmol) of N-[(1,1-dimethylethoxy)carbonyl]-glycine chloromethyl ester and 7.95 g (50 mmol) of sodium iodide in 50 ml of acetone was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 20% ethyl acetate/hexane) to obtain 2.04 g (78%) of the title compound.

¹H-NMR (CDCl₃, δ):1.48 (9H, s), 3.93 (2H, d, J=6 Hz), 4.97 (1H, br-s), 5.95 (2H, s)

### Reference Example 3

### 3-[[(1,1-dimethylethoxy)carbonyl]amino]-2-methylpropane Acid Chloromethyl Ester

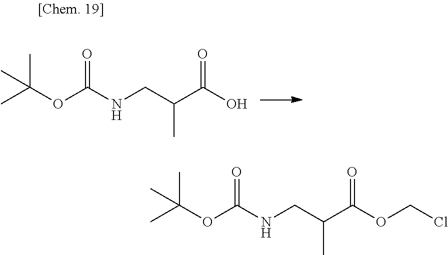

Under cooling on ice, a methylene chloride solution of 990 mg (6 mmol) of chloromethyl chlorosulfonate was added dropwise to a water 10 ml-methylene chloride 10 ml mixed solution of 1.02 g (5 mmol) of 3-[[(1,1-dimethylethoxy)carbonyl]amino]-2-methylpropane acid, 170 mg (0.5 mmol) of tetrabutylammonium hydrogen sulfate, and 1.68 g (20 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (10% to 20% ethyl acetate/hexane) to obtain 1.12 g (89%) of the title compound.

¹H-NMR (CDCl₃, δ):1.20 (3H, d, J=7 Hz), 1.43 (9H, s), 2.69-2.82 (1H, m), 3.20-3.41 (2H, m), 4.88 (1H, br-s), 5.69 (1H, d, J=6 Hz), 5.75 (1H, d, J=6 Hz)

### Reference Example 4

### 3-[[(1,1-dimethylethoxy)carbonyl]amino]-2-methylpropane Acid Iodomethyl Ester

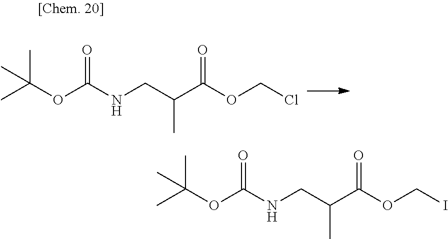

An acetone suspension of 1.12 g (8.9 mmol) of 3-[[(1,1-dimethylethoxy)carbonyl]amino]-2-methylpropane acid chloromethyl ester and 3.30 g (22 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 20% ethyl acetate/hexane) to obtain 1.21 g (79%) of the title compound.

¹H-NMR (CDCl₃, δ):1.16 (3H, d, J=7 Hz), 1.44 (9H, s), 2.67-2.78 (1H, m), 3.20-3.39 (2H, m), 4.87 (1H, br-s), 5.90 (1H, d, J=5 Hz), 5.95 (1H, d, J=5 Hz)

### Reference Example 5

### N-[(1,1-dimethylethoxy)carbonyl]-L-alanine Chloromethyl Ester

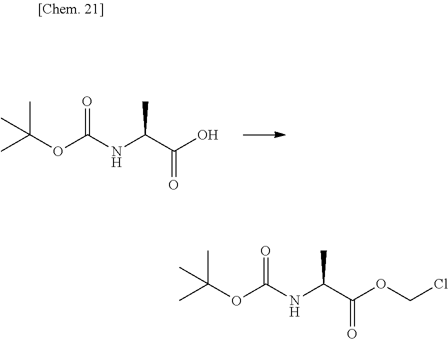

Under cooling on ice, a methylene chloride solution of 3.96 g (24 mmol) of chloromethyl chlorosulfonate was added dropwise to a water 40 ml-methylene chloride 40 ml mixed solution of 3.78 g (20 mmol) of N-[(1,1-dimethylethoxy)carbonyl]-L-alanine, 679 mg (2.0 mmol) of tetrabutylammonium hydrogen sulfate, and 6.72 g (80 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 4.33 g (91%) of the title compound.

¹H-NMR (CDCl₃, δ):1.41 (3H, d, J=7 Hz), 1.44 (9H, s), 4.31-4.42 (1H, m), 4.96 (1H, br-s), 5.65 (1H, d, J=5 Hz), 5.84 (1H, d, J=5 Hz)

### Reference Example 6

### N-[(1,1-dimethylethoxy)carbonyl]-L-alanine Iodomethyl Ester

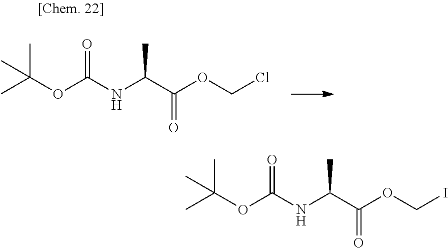

An acetone suspension of 4.33 g (18.2 mmol) of N-[(1,1-dimethylethoxy)carbonyl]-L-alanine chloromethyl ester and 15.0 g (0.1 mol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 20% ethyl acetate/hexane) to obtain 5.37 g (90%) of the title compound.

¹H-NMR (CDCl₃, δ):1.36 (3H, d, J=7 Hz), 1.43 (9H, s), 4.26-4.37 (1H, m), 4.95 (1H, br-s), 5.87 (1H, d, J=4 Hz), 6.03 (1H, d, J=4 Hz)

### Reference Example 7

### 3-[[(1,1-dimethylethoxy)carbonyl]amino]-2,2-dimethylpropane Acid Chloromethyl Ester

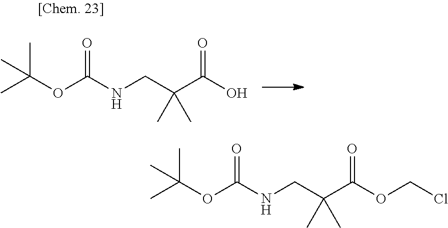

Under cooling on ice, a methylene chloride solution of 447 mg (2.7 mmol) of chloromethyl chlorosulfonate was added dropwise to a water 5 ml-methylene chloride 5 ml mixed solution of 491 mg (2.3 mmol) of 3-[[(1,1-dimethylethoxy)carbonyl]amino]-2,2-dimethylpropane acid, 77 mg (0.2 mmol) of tetrabutylammonium hydrogen sulfate, and 759 mg (9.0 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 563 mg (94%) of the title compound.

¹H-NMR (CDCl₃, δ):1.25 (6H, s), 1.43 (9H, s), 3.28 (2H, d, J=7 Hz), 4.88 (1H, br-s), 5.72 (2H, s)

### Reference Example 8

### 3-[[(1,1-dimethylethoxy)carbonyl]amino]-2,2-dimethylpropane Acid Iodomethyl Ester

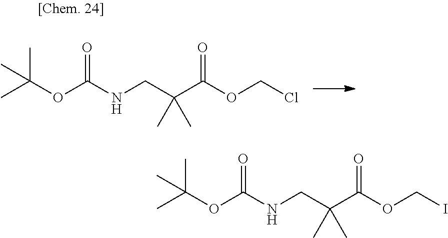

An acetone suspension of 563 mg (2.1 mmol) of 3-[[(1,1-dimethylethoxy)carbonyl]amino]-2,2-dimethylpropane acid chloromethyl ester and 1.69 g (11.3 mol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 20% ethyl acetate/hexane) to obtain 588 mg (68%) of the title compound.

¹H-NMR (CDCl₃, δ):1.16 (6H, s), 1.43 (9H, s), 3.26 (2H, d, J=7 Hz), 4.88 (1H, br-s), 5.93 (2H, s)

### Reference Example 9

### N-[(1,1-dimethylethoxy)carbonyl]-glycine 1-chloroethyl Ester

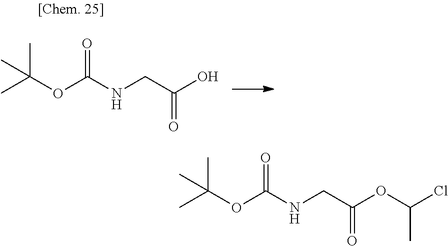

Under cooling on ice, a methylene chloride solution of 5.01 g (28 mmol) of 1-chloroethyl chlorosulfonate was added dropwise to a water 40 ml-methylene chloride 40 ml mixed solution of 3.50 g (20 mmol) of N-[(1,1-dimethylethoxy)carbonyl]glycine, 679 mg (2 mmol) of tetrabutylammonium hydrogen sulfate, and 6.72 g (80 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (10% to 20% ethyl acetate/hexane) to obtain 3.49 g (74%) of the title compound.

¹H-NMR (CDCl₃, δ):1.44 (9H, s), 1.80 (3H, d, J=6 Hz), 3.89 (1H, dd, J=19 Hz, 5 Hz), 4.02 (1H, dd, J=19 Hz, 6 Hz), 4.97 (1H, br-s), 6.57 (1H, q, J=6 Hz)

### Reference Example 10

### 1-[[[(1,1-dimethylethoxy)carbonyl]amino]methyl]cyclopentane Carboxylic Acid

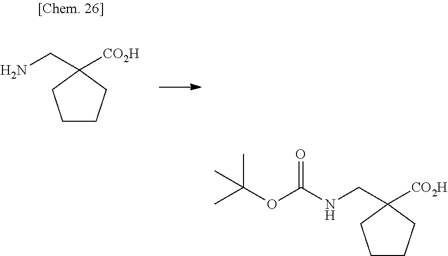 [SMILES: NCC1(C(=O)O)CCCC1 ; CC(C)(C)OC(=O)NCC1(C(=O)O)CCCC1]

601 mg (2.8 mmol) of tert-butyldicarbonate was added to a water-dioxane mixed solution of 500 mg (2.8 mmol) of 1-(aminomethyl)cyclopentane carboxylic acid and 844 mg (8.3 mmol) of triethylamine and stirred at room temperature overnight. The reaction solution was condensed under reduced pressure, and the residue was dissolved in ethyl acetate and washed with a 10% potassium hydrogensulfate solution and saturated saline. The organic layer was dried with anhydrous sodium sulfate, and the solvent was distilled off under reduced pressure to obtain 69 mg (quantitative) of the title compound.

¹H-NMR (CDCl₃, δ):1.44 (9H, s), 1.58-1.81 (6H, m), 1.96-2.07 (2H, m), 3.28 (2H, d, J=6 Hz), 5.10 (1H, br-s)

### Reference Example 11

### 1-[[[(1,1-dimethylethoxy)carbonyl]amino]methyl]cyclopentane Carboxylic Acid Chloromethyl Ester

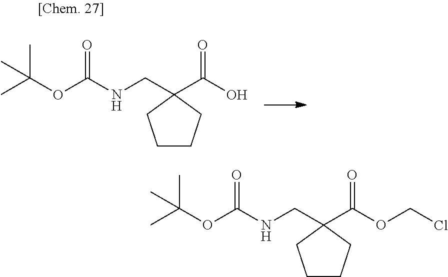

Under cooling on ice, a methylene chloride solution of 561 mg (3.4 mmol) of chloromethyl chlorosulfonate was added dropwise to a water 6 ml-methylene chloride 6 ml mixed solution of 610 mg (2.8 mmol) of 1-[[[(1,1-dimethylethoxy)carbonyl]amino]methyl]cyclopentane carboxylic acid, 95 mg (0.28 mmol) of tetrabutylammonium hydrogen sulfate, and 941 mg (11.2 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and then the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 756 mg (93%) of the title compound.

¹H-NMR (CDCl₃, δ):1.43 (9H, s), 1.60-1.79 (6H, m), 1.92-2.03 (2H, m), 3.32 (2H, d, J=7 Hz), 4.96 (1H, s), 5.73 (2H, s)

### Reference Example 12

### 1-[[[(1,1-dimethylethoxy)carbonyl]amino]methyl]cyclopentane Carboxylic Acid Iodomethyl Ester

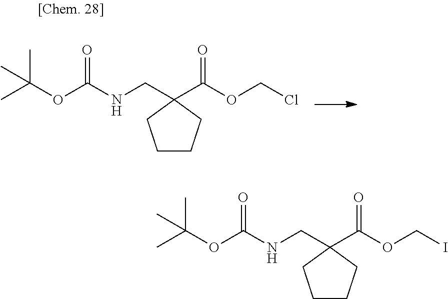

An acetone suspension of 756 mg (2.6 mmol) of 1-[[[(1,1-dimethylethoxy)carbonyl]amino]methyl]cyclopentane carboxylic acid chloromethyl ester and 2.1 g (14 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (5% ethyl acetate/hexane) to obtain 591 mg (59%) of the title compound.

¹H-NMR (CDCl₃, δ):1.43 (9H, s), 1.57-1.65 (2H, m), 1.68-1.76 (4H, m), 1.91-1.99 (2H, m), 3.30 (2H, d, J=7 Hz), 4.95 (1H, br-s), 5.94 (2H, s)

### Reference Example 13

### 1-[[[(1,1-dimethylethoxy)carbonyl]amino]methyl]cyclopropanecarboxylic Acid Ethyl Ester

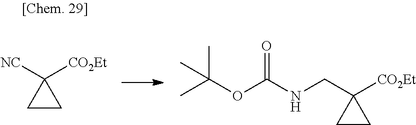 [SMILES: CCOC(=O)C1(C#N)CC1 ; CCOC(=O)C1(CNC(=O)OC(C)(C)C)CC1]

6.84 g (28.8 mmol) of cobalt chloride hexahydrate was added to 100 ml of methanol of 2.0 g (14.4 mmol) of 1-cyanocyclopropanecarboxylic acid ethyl ester. While cooling at a water bath, 5.44 g (143.7 mmol) of sodium borohydride was added in small portions to the mixed liquid, and then stirred at room temperature for 30 minutes. 237 ml of 2 N hydrochloric acid was added to the reaction solution and stirred at room temperature for 2 hours. 57.7 g (569 mmol) of triethylamine was added to the reaction solution and stirred for 1 hour, and then 3.27 g (15 mmol) of tert-butyldicarbonate was added thereto and stirred at room temperature overnight. Impurities were filtered and washing with ethyl acetate was performed three times. The obtained filtrate was combined, the organic layer was isolated, and then the water layer portion was extracted with ethyl acetate. The organic layer was combined, washed with saturated saline, and then dried with anhydrous sodium sulfate, and the solvent was distilled off under reduced pressure. The residue was purified by silica gel column chromatography (5 to 10% ethyl acetate/hexane) to obtain 2.00 g (57%) of the title compound.

¹H-NMR (CDCl₃, δ):0.88-0.97 (2H, m), 1.17-1.25 (2H, m), 1.23 (3H, t, J=7 Hz), 1.44 (9H, s), 3.28 (2H, d, J=6 Hz), 4.12 (2H, q, J=7 Hz), 5.16 (1H, br-s)

### Reference Example 14

### 1-[[[(1,1-dimethylethoxy)carbonyl]amino]methyl]cyclopropanecarboxylic Acid

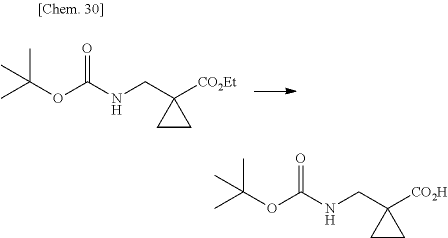 [SMILES: CCOC(=O)C1(CNC(=O)OC(C)(C)C)CC1 ; CC(C)(C)OC(=O)NCC1(C(=O)O)CC1]

20 ml of 2 N sodium hydroxide aqueous solution was added to a solution of 2.00 g (8.2 mmol) of 1-[[[(1,1-dimethylethoxy)carbonyl]amino]methyl]cyclopropanecarboxylic acid ethyl ester in 30 ml of tetrahydrofuran and was heated under reflux for 4 hours. The reaction solution was condensed under reduced pressure, diethyl ether was then added to the residue, and extraction with water was performed two times. 6.13 g (45 mmol) of potassium hydrogensulfate was added to the water layer to become acidic, and then extracted with ethyl acetate. The organic layer was washed with saturated saline, and the solvent was distilled off under reduced pressure to obtain 1.73 g (98%) of the title compound.

¹H-NMR (CDCl₃, δ):1.00-1.11 (2H, m), 1.27-1.34 (2H, m), 1.43 (9H, s), 3.28 (2H, d, J=6 Hz), 5.19 (1H, br-s)

### Reference Example 15

### 1-[[[(1,1-dimethylethoxy)carbonyl]amino]methyl]cyclopropanecarboxylic Acid Chloromethyl Ester

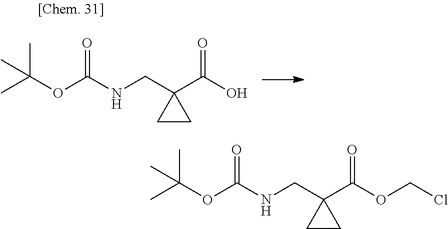

Under cooling on ice, a methylene chloride solution of 1.59 g (9.6 mmol) of chloromethyl chlorosulfonate was added dropwise to a water 8 ml-methylene chloride 8 ml mixed solution of 1.73 g (8 mmol) of 1-[[[(1,1-dimethylethoxy)carbonyl]amino]methyl]cyclopropanecarboxylic acid, 272 mg (0.8 mmol) of tetrabutylammonium hydrogen sulfate, and 2.69 g (32 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 1.95 g (92%) of the title compound.

¹H-NMR (CDCl₃, δ):1.03-1.12 (2H, m), 1.29-1.36 (2H, m), 1.44 (9H, s), 3.32 (2H, d, J=6 Hz), 5.14 (1H, br-s), 5.71 (2H, s)

### Reference Example 16

### 1-[[[(1,1-dimethylethoxy)carbonyl]amino]methyl]cyclopropanecarboxylic Acid Iodomethyl Ester

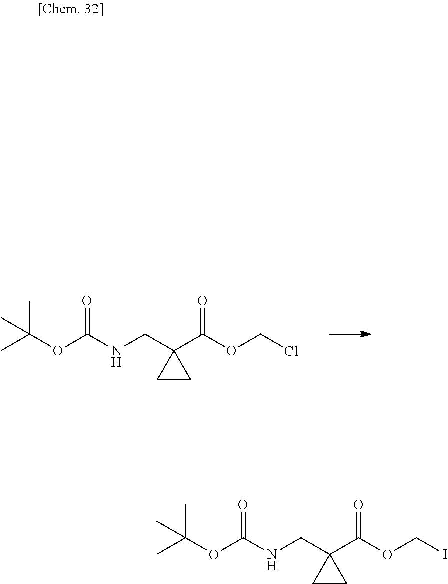 [SMILES: CC(C)(C)OC(=O)NCC1(C(=O)OCCl)CC1 ; CC(C)(C)OC(=O)NCC1(C(=O)OCI)CC1]

An acetone suspension of 1.95 g (7.4 mmol) of 1-[[[(1,1 l-dimethylethoxy)carbonyl]amino]methyl]cyclopropanecarboxylic acid chloromethyl ester and 5.55 g (37 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (5% to 10% ethyl acetate/hexane) to obtain 1.38 g (53%) of the title compound.

¹H-NMR (CDCl₃, δ):0.98-1.05 (2H, m), 1.23-1.31 (2H, m), 1.44 (9H, s), 3.30 (2H, d, J=6 Hz), 5.11 (1H, br-s), 5.91 (2H, s)

### Reference Example 17

### N-[(1,1-dimethylethoxy)carbonyl]-β-alanine Chloromethyl Ester

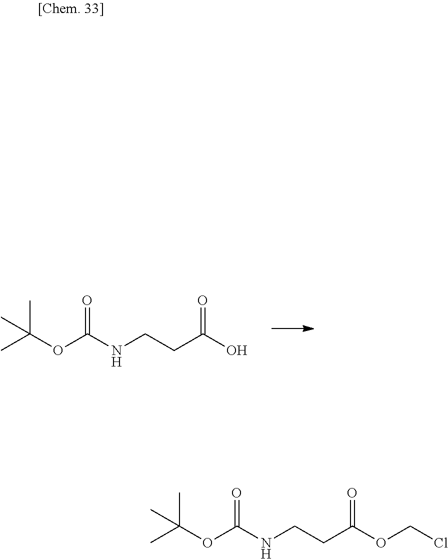

Under cooling on ice, a methylene chloride solution of 1.15 g (7.0 mmol) of chloromethyl chlorosulfonate was added dropwise to a water 10 ml-methylene chloride 10 ml mixed solution of 1.10 g (5.8 mmol) of N-[(1,1-dimethylethoxy)carbonyl]-β-alanine, 197 mg (0.58 mmol) of tetrabutylammonium hydrogen sulfate, and 1.95 g (23.2 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and then the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 1.20 g (87%) of the title compound.

¹H-NMR(CDCl₃, δ):1.44 (9H, s), 2.62 (2H, t, J=6 Hz), 3.37-3.46 (2H, m), 4.95 (1H, br-s), 5.71 (2H, s)

### Reference Example 18

### N-[(1,1-dimethylethoxy)carbonyl]-β-alanine Iodomethyl Ester

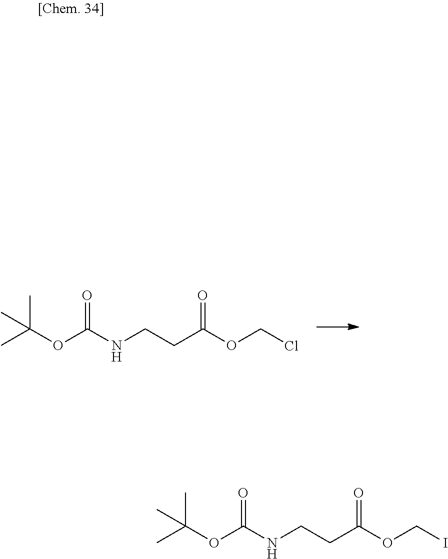

An acetone suspension of 1.20 g (5.1 mmol) of N-[(1,1-dimethylethoxy)carbonyl]-β-alanine chloromethyl ester and 4.35 g (29 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 30% ethyl acetate/hexane) to obtain 1.33 g (80%) of the title compound.

¹H-NMR (CDCl₃, δ):1.44 (9H, s), 2.57 (2H, t, J=6 Hz), 3.36-3.45 (2H, m), 4.95 (1H, br-s), 5.91 (2H, s)

### Reference Example 19

### 2-[[[(1,1-dimethylethoxy)carbonyl]amino]methyl]-3-methylbutanoic Acid Ethyl Ester

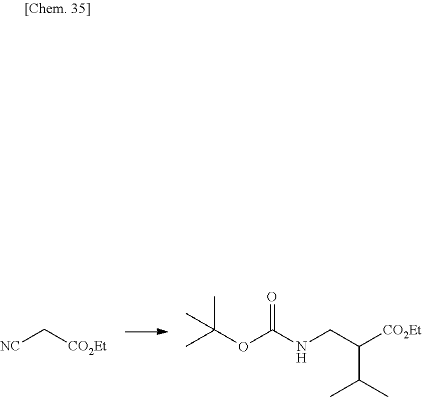 [SMILES: CCOC(=O)CC#N ; CCOC(=O)C(CNC(=O)OC(C)(C)C)C(C)C]

1.38 g (60 mmol) of metallic sodium was added to 60 ml of dry ethanol and dissolved, and then 5.66 g (50 mmol) of cyano ethyl acetate was added thereto at room temperature and stirred for 15 minutes. A solution of 10.71 g (63 mmol) of 2-iodopropane in 15 ml of dry ethanol was slowly added to the reaction solution at room temperature and stirred for 3 hours. The reaction solution was heated under reflux for 1 hour and then cooled to room temperature, and 10% sodium hydrogensulfate was added thereto to stop the reaction. Diethyl ether was added to the reaction solution, and the water layer was extracted with diethyl ether. The organic layer was combined and washing with 10% sodium thiosulfate and saturated saline was performed. The organic layer was dried with anhydrous sodium sulfate, and the solvent was distilled off under reduced pressure to obtain a crude 2-cyano-3-methylbutanoic acid ethyl ester. The obtained crude 2-cyano-3-methylbutanoic acid ethyl ester was dissolved in 200 ml of methanol, and 23.8 g (0.1 mol) of cobalt chloride hexahydrate was added thereto. While cooling at a water bath, 18.9 g (0.5 mol) of sodium borohydride was added in small portions to the mixed liquid, and then stirred at room temperature for 30 minutes. Under cooling on ice, 200 ml of 6 N hydrochloric acid and 225 ml of 2 N hydrochloric acid were added to the reaction solution and stirred for 2 hours at room temperature. 200 g (1.98 mol) of triethylamine was added to the reaction solution and stirred for 1 hour, and then 11.35 g (52 mmol) of tert-butyldicarbonate was added thereto and stirred at room temperature overnight. Impurities were filtered and washing with ethyl acetate was performed three times. The obtained filtrate was combined, the organic layer was isolated, and then the water layer portion was extracted with ethyl acetate. The organic layer was combined and washed with saturated saline. The organic layer was dried with anhydrous sodium sulfate, and then the solvent was distilled off anhydrous sodium sulfate. The residue was purified by silica gel column chromatography (3% to 10% ethyl acetate/hexane) to obtain 7.02 g (54%) of the title compound.

¹H-NMR (CDCl₃, δ):0.95 (3H, d, J=7 Hz), 0.98 (3H, d, J=7 Hz), 1.27 (3H, d, J=7 Hz), 1.44 (9H, s), 1.90-2.00 (1H, m), 2.35-2.48 (1H, m), 3.13-3.47 (2H, m), 4.11-4.23 (2H, m), 4.82 (1H, br-s)

### Reference Example 20

### 2-[[[(1,1-dimethylethoxy)carbonyl]amino]methyl]3-methyl-butanoic Acid Chloromethyl Ester

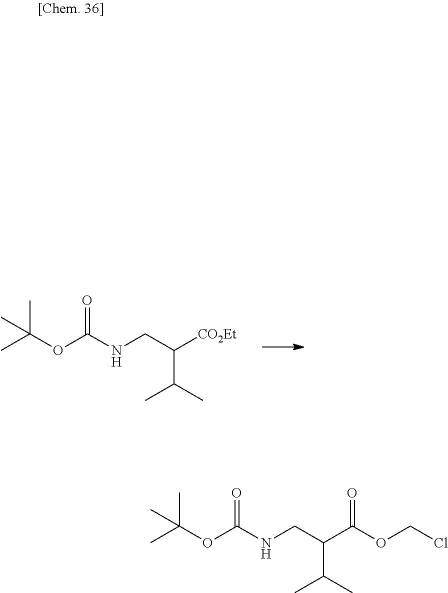 [SMILES: CCOC(=O)C(CNC(=O)OC(C)(C)C)C(C)C ; CC(C)C(CNC(=O)OC(C)(C)C)C(=O)OCCl]

56 ml of 2 N sodium hydroxide aqueous solution was added to a solution of 7.02 g (27 mmol) of 2-[[[(1,1-dimethylethoxy)carbonyl]amino]methyl]-3-methylbutanoic acid ethyl ester in 50 ml of tetrahydrofuran and was heated under reflux overnight. The reaction solution was condensed under reduced pressure, diethyl ether was added to the residue, and extraction with water was performed two times. Potassium hydrogensulfate was added to the water layer to become acidic, and then extracted with ethyl acetate. The organic layer was washed with saturated saline, and the solvent was distilled off under reduced pressure. Hexane was added to the residue and stirred, and the precipitated crystals were filtered to obtain 4.19 g (68%) of 2-[[[(1,1-dimethylethoxy)carbonyl]amino]methyl]-3-methylbutanoic acid. A methylene chloride solution of 3.58 g (22 mmol) of chloromethyl chlorosulfonate was added dropwise under cooling on ice to a water 40 ml-methylene chloride 40 ml mixed solution of 4.19 g (18 mmol) of the obtained 2-[[[(1,1-dimethylethoxy)carbonyl]amino]methyl]-3-methylbutanoic acid, 615 mg (1.8 mmol) of tetrabutylammonium hydrogen sulfate, and 6.08 g (72 mmol) of sodium hydrogen carbonate. The reaction solution was warmed to room temperature and stirred overnight. The methylene chloride layer of the reaction solution was isolated and washed with saturated saline, the organic layer was then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 4.78 g (94%) of the title compound.

¹H-NMR (CDCl₃, δ):0.98 (3H, d, J=7 Hz), 0.99 (3H, d, J=7 Hz), 1.43 (9H, s), 1.94-2.06 (1H, m), 2.45-2.59 (1H, m), 3.18-3.52 (2H, m), 4.77 (1H, br-s), 5.68 (1H, d, J=6 Hz), 5.79 (1H, d, J=6 Hz)

### Reference Example 21

### 2-[[[(1,1-dimethylethoxy)carbonyl]amino]methyl]-3-methylbutanoic Acid Iodomethyl Ester

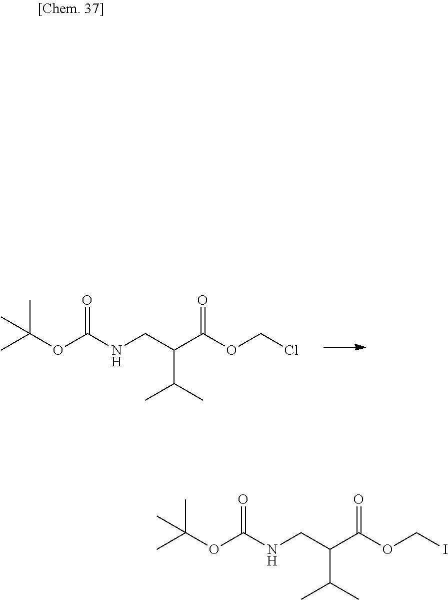 [SMILES: CC(C)C(CNC(=O)OC(C)(C)C)C(=O)OCCl ; CC(C)C(CNC(=O)OC(C)(C)C)C(=O)OCI]

An acetone suspension of 4.78 g (17.1 mmol) of 2-[[[(1,1-dimethylethoxy)carbonyl]amino]methyl]-3-methylbutanoic acid chloromethyl ester and 12.7 g (85.5 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (6% to 10% ethyl acetate/hexane) to obtain 5.00 g (79%) of the title compound.

¹H-NMR (CDCl₃, δ):0.98 (6H, d, J=7 Hz), 1.43 (9H, s), 1.92-2.04 (1H, m), 2.47-2.55 (1H, m), 3.18-3.28 (1H, m), 3.42-3.52 (1H, m), 4.77 (1H, br-s), 5.89 (1H, d, J=5 Hz), 5.97 (1H, d, J=5 Hz)

### Reference Example 22

### 1-[[(1,1-dimethylethoxy)carbonyl]amino]cyclopropanecarboxylic Acid Chloromethyl Ester

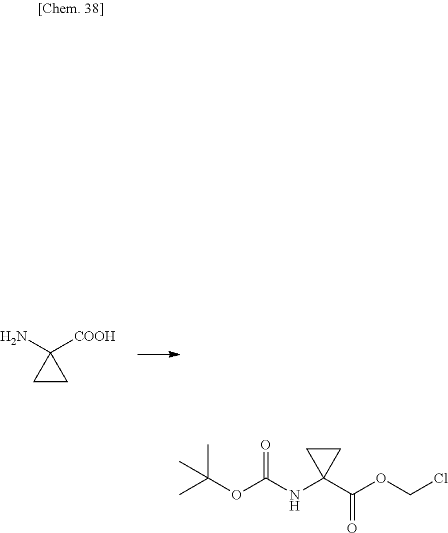 [SMILES: NC1(C(=O)O)CC1 ; CC(C)(C)OC(=O)NC1(C(=O)OCCl)CC1]

10.79 g (49.5 mmol) of tert-butyldicarbonate was added to a water-dioxane mixed solution of 5.0 g (49.5 mmol) of 1-aminocyclopropane carboxylic acid and 10.0 g (98.9 mmol) of triethylamine and stirred at room temperature overnight. The reaction solution was condensed under reduced pressure, and the residue was dissolved in ethyl acetate and washed with a 10% potassium hydrogensulfate solution and saturated saline. The organic layer was dried with anhydrous sodium sulfate, and then the solvent was distilled off under reduced pressure. Hexane was added to the residue and stirred, and the precipitated crystals were filtered to obtain 9.23 g (93%) of 1-[[(1,1-dimethylethoxy)carbonyl]amino]cyclopropanecarboxylic acid. A methylene chloride solution of 1.98 g (12 mmol) of chloromethyl chlorosulfonate was added dropwise under cooling on ice to a water 20 ml-methylene chloride 20 ml mixed solution of 2.01 g (10 mmol) of the obtained 1-[[(1,1-dimethylethoxy)carbonyl]amino]cyclopropanecarboxylic acid, 340 mg (1 mmol) of tetrabutylammonium hydrogen sulfate, and 3.36 g (40 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 2.41 g (96%) of the title compound.

¹H-NMR (CDCl₃, δ):1.27 (2H, br-s), 1.45 (9H, s), 1.61 (2H, br-s), 5.13 (1H, br-s), 5.71 (2H, s)

### Reference Example 23

### 1-[[(1,1-dimethylethoxy)carbonyl]amino]cyclopropanecarboxylic Acidiodomethyl Ester

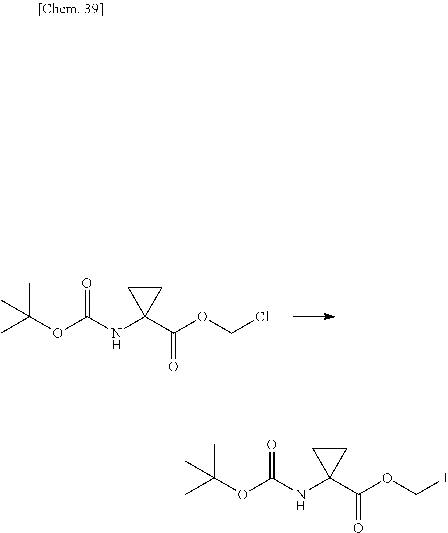 [SMILES: CC(C)(C)OC(=O)NC1(C(=O)OCCl)CC1 ; CC(C)(C)OC(=O)NC1(C(=O)OCI)CC1]

An acetone suspension of 2.41 g (9.6 mmol) of 1-[[(1,1-dimethylethoxy)carbonyl]amino]cyclopropanecarboxylic acid chloromethyl ester and 7.20 g (48 mmol) of sodium iodide was heated under reflux for 1 hour under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (10% to 20% ethyl acetate/hexane) to obtain 2.78 g (85%) of the title compound.

¹H-NMR (CDCl₃, δ):1.22 (2H, br-s), 1.46 (9H, s), 1.51-1.62 (2H, m), 5.12 (1H, br-s), 5.92 (2H, s)

### Reference Example 24

### 2-cyano-3,3-dimethylbutanoic Acid Ethyl Ester

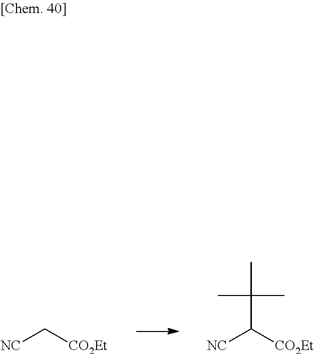

70 ml (60 mmol) of 0.86 mol/L diethylaluminum chloride hexane solution was added dropwise to a solution of 6.80 g (60 mmol) of cyano ethyl acetate in 60 ml of dry toluene at 35° C. to 45° C. The reaction solution was stirred at the same temperature for 30 minutes, a solution of 5.55 g (60 mmol) of tert-butyl chloride in 30 ml of dry toluene was then added dropwise thereto and stirred at the same temperature for 2 hours. The reaction solution was slowly added to 74 ml of 15% hydrochloric acid at 20° C. to 50° C. and then stirred at room temperature for 1 hour. The reaction solution was stirred at 60° C. for 1 hour and then cooled to room temperature, the organic layer was isolated, and the water layer was extracted with toluene. The organic layer was combined and washed with a saturated sodium hydrogen carbonate aqueous solution and saturated saline, the organic layer was then dried with anhydrous sodium sulfate, and the solvent was distilled off under reduced pressure to obtain 8.26 g (81%) of the title compound.

¹H-NMR (CDCl₃, δ):1.18 (9H, s), 1.32 (3H, t, J=7 Hz), 3.27 (1H, s), 4.19-4.32 (2H, m)

### Reference Example 25

### 2-[[[(1,1-dimethylethoxy)carbonyl]amino]methyl]-3,3-dimethylbutanoic Acid Ethyl Ester

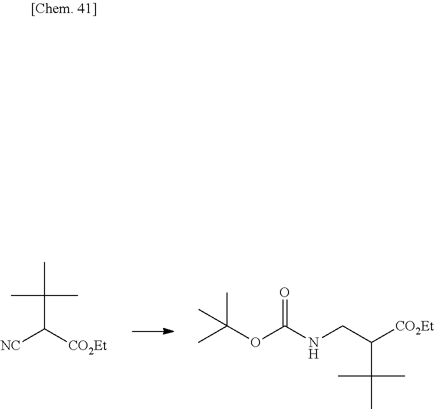 [SMILES: CCOC(=O)C(C#N)C(C)(C)C ; CCOC(=O)C(CNC(=O)OC(C)(C)C)C(C)(C)C]

23.2 g (97.6 mol) of cobalt chloride hexahydrate was added to a solution of P 8.26 g (48.8 mmol) of 2-cyano-3,3-dimethylbutanoic acid ethyl ester in 200 ml of methanol. While cooling at a water bath, 18.5 g (488 mol) of sodium borohydride was added in small portions to the mixed liquid, and then stirred at room temperature for 30 minutes. Under cooling on ice, 195 ml of 6 N hydrochloric acid and 220 ml of 2 N hydrochloric acid were added to the reaction solution and stirred for 2 hours at room temperature. 196 g (1.93 mol) of triethylamine was added to the reaction solution and stirred for 1 hour, and then 11.1 g (51 mmol) of tert-butyldicarbonate was added thereto and stirred at room temperature overnight. Impurities were filtered and washing with ethyl acetate was performed three times. The obtained filtrate was combined, the organic layer was isolated, and then the water layer portion was extracted with ethyl acetate. The organic layer was combined, washed with saturated saline, and then dried with anhydrous sodium sulfate, and the solvent was distilled off under reduced pressure. The residue was purified by silica gel column chromatography (3% to 10% ethyl acetate/hexane) to obtain 9.91 g (74%) of the title compound.

¹H-NMR (CDCl₃, δ):0.99 (9H, s), 1.27 (3H, t, J=7 Hz), 1.44 (9H, s), 2.45-2.54 (1H, m), 3.17-3.27 (1H, m), 3.45-3.54 (1H, m), 4.17 (2H, q, J=7 Hz), 4.43 (1H, br-s)

### Reference Example 26

### 2-[[[(1,1-dimethylethoxy)carbonyl]amino]methyl]-3,3-dimethylbutanoic Acid Chloromethyl Ester

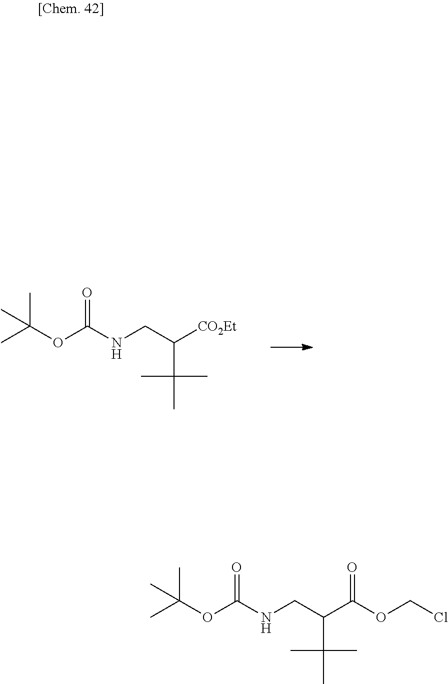 [SMILES: CCOC(=O)C(CNC(=O)OC(C)(C)C)C(C)(C)C ; CC(C)(C)OC(=O)NCC(C(=O)OCCl)C(C)(C)C]

5.6 g (84.8 mmol) of potassium hydroxide was added to a water 50 ml-methanol 50 ml mixed solution of 6.87 g (25.1 mmol) of 2-[[[(1,1-dimethylethoxy)carbonyl]amino]methyl]-3,3-dimethylbutanoic acid ethyl ester and was heated under reflux for 30 hours. The reaction solution was condensed under reduced pressure, diethyl ether was then added to the residue, and extraction with water was performed two times. Potassium hydrogensulfate was added to the water layer to become acidic, and then extracted with ethyl acetate. The organic layer was washed with saturated saline, and the solvent was distilled off under reduced pressure. Hexane was added to the residue and stirred, and then the precipitated crystals were filtered to obtain 4.01 g (65%) of 2-[[[(1,1-dimethylethoxy)carbonyl]amino]methyl]-3,3-dimethylbutanoic acid. A methylene chloride solution of 847 mg (5.14 mmol) of chloromethyl chlorosulfonate was added dropwise under cooling on ice to a water 10 ml-methylene chloride 10 ml mixed solution of 1.05 g (4.28 mmol) of the obtained 2-[[[(1,1-dimethylethoxy)carbonyl]amino]methyl]-3,3-dimethylbutanoic acid, 145 mg (0.4 mmol) of tetrabutylammonium hydrogen sulfate, and 1.44 g (17.1 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 1.16 g (92%) of the title compound.

¹H-NMR (CDCl₃, δ):1.02 (9H, s), 1.42 (9H, s), 2.59-2.68 (1H, m), 3.19-3.29 (1H, m), 3.49-3.59 (1H, m), 4.64 (1H, br-s), 5.68 (1H, d, J=6 Hz), 5.79 (1H, d, J=6 Hz)

### Reference Example 27

### 2-[[[(1,1-dimethylethoxy)carbonyl]amino]methyl]-3,3-dimethylbutanoic Acid Iodomethyl Ester

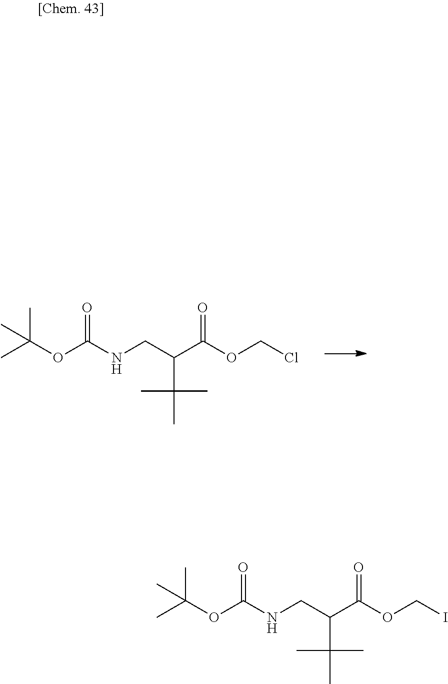 [SMILES: CC(C)(C)OC(=O)NCC(C(=O)OCCl)C(C)(C)C ; CC(C)(C)OC(=O)NCC(C(=O)OCI)C(C)(C)C]

An acetone suspension of 1.16 g (3.96 mmol) of 2-[[[(1,1-dimethylethoxy)carbonyl]amino]methyl]-3,3-dimethylbutanoic acid chloromethyl ester and 2.96 g (19.8 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (6% to 10% ethyl acetate/hexane) to obtain 1.29 g (85%) of the title compound.

¹H-NMR (CDCl₃, δ):1.01 (9H, s), 1.43 (9H, s), 2.55-2.64 (1H, m), 3.18-3.28 (1H, m), 3.50-3.60 (1H, m), 4.63 (1H, br-s), 5.90 (1H, d, J=4 Hz), 5.94 (1H, d, J=4 Hz)

### Reference Example 28

### N-[(1,1-dimethylethoxy)carbonyl)]-L-valine Chloromethyl Ester

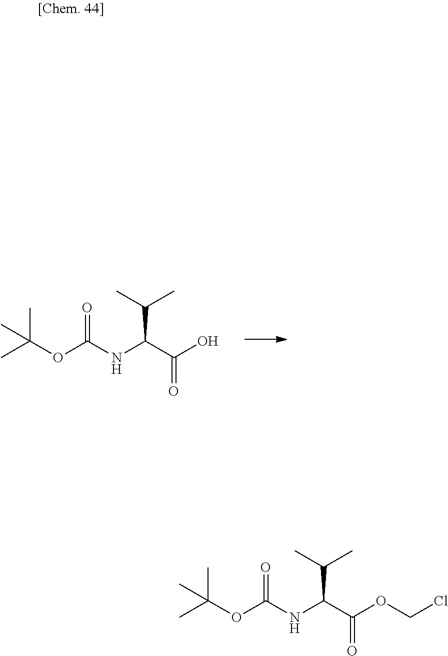 [SMILES: CC(C)[C@H](NC(=O)OC(C)(C)C)C(=O)O ; CC(C)[C@H](NC(=O)OC(C)(C)C)C(=O)OCCl]

Under cooling on ice, a methylene chloride solution of 825 mg (6.0 mmol) of chloromethyl chlorosulfonate was added dropwise to a water 10 ml-methylene chloride 10 ml mixed solution of 1.09 (5.0 mmol) of N-[(1,1-dimethylethoxy)carbonyl)]-L-valine, 170 mg (0.5 mmol) of tetrabutylammonium hydrogen sulfate, and 1.68 g (20.0 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and then the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 1.26 gg (95%) of the title compound.

¹H-NMR (CDCl₃, 8):0.93 (3H, d, J=7 Hz), 1.01 (3H, d, J=7 Hz), 1.45 (9H, s), 2.11-2.224 (1H, m), 4.21-4.31 (1H, m), 4.97 (1H, br-s), 5.62 (1H, d, J=6 Hz), 5.88 (1H, d, J=6 Hz)

### Reference Example 29

### N-[(1,1-dimethylethoxy)carbonyl)]-L-valine Iodomethyl Ester

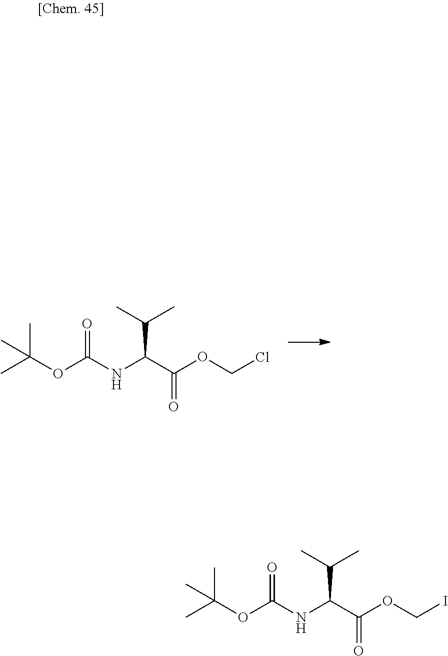 [SMILES: CC(C)[C@H](NC(=O)OC(C)(C)C)C(=O)OCCl ; CC(C)[C@H](NC(=O)OC(C)(C)C)C(=O)OCI]

An acetone suspension of 1.26 g (4.72 mmol) of N-[(1,1-dimethylethoxy)carbonyl)]-L-valine chloromethyl ester and 3.54 g (23.6 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (5% to 10% ethyl acetate/hexane) to obtain 1.47 g (87%) of the title compound.

¹H-NMR (CDCl₃, δ):0.92 (3H, d, J=7 Hz), 1.00 (3H, d, J=7 Hz), 1.46 (9H, s), 2.11-2.23 (1H, m), 4.17-4.26 (1H, m), 4.95 (1H, d, J=8 Hz), 9.85 (1H, d, J=5 Hz), 6.04 (1H, d, J=5 Hz)

### Reference Example 30

### N-[(1,1-dimethylethoxy)carbonyl)]-L-tert-leucine Chloromethyl Ester

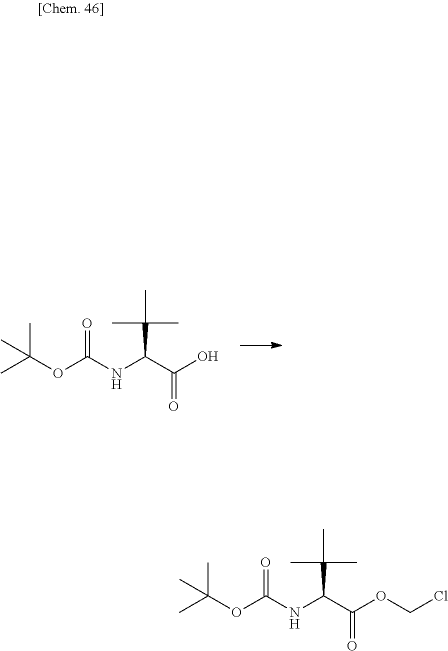 [SMILES: CC(C)(C)OC(=O)N[C@H](C(=O)O)C(C)(C)C ; CC(C)(C)OC(=O)N[C@H](C(=O)OCCl)C(C)(C)C]

Under cooling on ice, a methylene chloride solution of 825 mg (6.0 mmol) of chloromethyl chlorosulfonate was added dropwise to a water 10 ml-methylene chloride 10 ml mixed solution of 1.16 (5.0 mmol) of N-[(1,1-dimethylethoxy)carbonyl)]-L-tert-leucine, 170 mg (0.5 mmol) of tetrabutylammonium hydrogen sulfate, and 1.68 g (20.0 mmol) of sodium hydrogen carbonate. The reaction solution was warmed to room temperature and stirred overnight. The methylene chloride layer of the reaction solution was isolated, washed with saturated saline, and then dried with anhydrous sodium sulfate, and the solvent was distilled off under reduced pressure. The residue was dissolved again in diethyl ether and washed with water. The water layer was extracted with a small amount of diethyl ether and combined with the organic layer. The organic layer was washed with saturated saline and dried with anhydrous sodium sulfate, and the solvent was distilled off under reduced pressure to obtain 1.31 gg (94%) of the title compound.

¹H-NMR (CDCl₃, δ):1.02 (9H, s), 1.45 (9H, s), 4.13 (1H, d, J=8 Hz), 5.05 (1H, d, J=8 Hz), 5.61 (1H, d, J=6 Hz), 5.88 (1H, d, J=6 Hz)

### Reference Example 31

### N-[(1,1-dimethylethoxy)carbonyl)]-L-tert-leucine Iodomethyl Ester

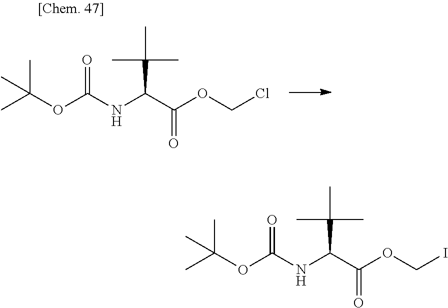 [SMILES: CC(C)(C)OC(=O)N[C@H](C(=O)OCCl)C(C)(C)C ; CC(C)(C)OC(=O)N[C@H](C(=O)OCI)C(C)(C)C]

An acetone suspension of 1.31 g (4.68 mmol) of N-[(1,1-dimethylethoxy)carbonyl)]-L-tert-leucine chloromethyl ester and 3.50 g (23.4 mmol) of sodium iodide was heated under reflux for 2 hours under shading. The reaction solution was cooled to room temperature and condensed under reduced pressure. Diethyl ether was added to the residue and stirred, impurities were then removed by filtration, and the filtrate was washed with a 10% sodium thiosulfate aqueous solution and saturated saline. The organic layer was dried with anhydrous sodium sulfate, and then the solvent was distilled off under reduced pressure. The residue was purified by silica gel column chromatography (5% to 10% ethyl acetate/hexane) to obtain 1.51 g (86%) of the title compound.

¹H-NMR (CDCl₃, δ):1.01 (9H, s), 1.44 (9H, s), 4.09 (1H, d, J=8 Hz), 5.04 (1H, d, J=8 Hz), 5.83 (1H, d, J=6 Hz), 6.02 (1H, d, J=6 Hz)

### Reference Example 32

### N-[(1,1-dimethylethoxy)carbonyl)]-3-alanine 1-chloroethyl Ester

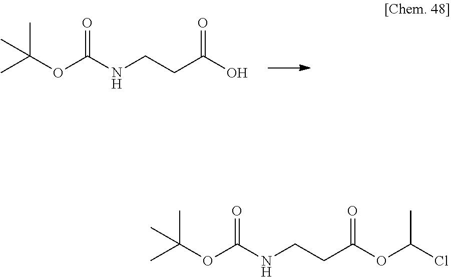 [SMILES: CC(C)(C)OC(=O)NCCC(=O)O ; CC(Cl)OC(=O)CCNC(=O)OC(C)(C)C]

Under cooling on ice, a methylene chloride solution of 10.74 g (60 mmol) of 1-chloroethyl chlorosulfonate was added dropwise to a water 100 ml-methylene chloride 100 ml mixed solution of 9.46 g (50 mmol) of N-[(1,1-dimethylethoxy)carbonyl]-3-alanine, 1.70 g (5 mmol) of tetrabutylammonium hydrogen sulfate, and 16.8 g (0.2 mol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (10% to 20% ethyl acetate/hexane) to obtain 6.88 g (55%) of the title compound.

¹H-NMR (CDCl₃, δ):1.44 (9H, s), 1.79 (3H, d, J=6 Hz), 2.58 (2H, t, J=6 Hz), 3.42 (2H, dd, J=12 Hz, 6 Hz), 4.96 (1H, br-s), 6.54 (1H, q, J=6 Hz)

### Reference Example 33

### 2-[[[(1,1-dimethylethoxy)carbonyl]amino]methyl]-2-ethylbutanoic Acid Chloromethyl Ester

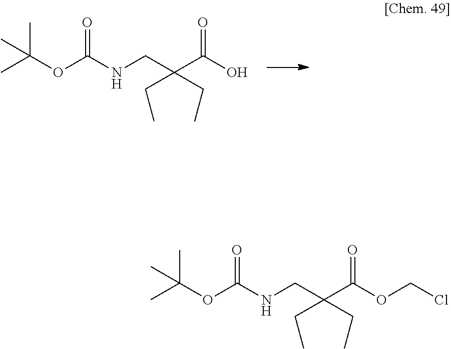 [SMILES: CCC(CC)(CNC(=O)OC(C)(C)C)C(=O)O ; CCC(CC)(CNC(=O)OC(C)(C)C)C(=O)OCCl]

Under cooling on ice, a methylene chloride solution of 1.49 g (10.02 mmol) of chloromethyl chlorosulfonate was added dropwise to a water 20 ml-methylene chloride 20 ml mixed solution of 1.23 g (5.01 mmol) of 2-[[[(1,1-dimethylethoxy)carbonyl]amino]methyl]-2-ethylbutanoic acid, 170 mg (0.50 mmol) of tetrabutylammonium hydrogen sulfate, and 3.4 g (40 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (5% to 40% ethyl acetate/hexane) to obtain 1.31 g (89%) of the title compound.

¹H-NMR (CDCl₃, δ):0.87 (6H, t, J=8 Hz), 1.43 (9H, s), 1.64 (4H, q, J=8 Hz), 3.36 (2H, d, J=7 Hz), 4.72 (1H, br-s), 5.74 (2H, s)

### Reference Example 34

### 1-[[(1,1-dimethylethoxy)carbonyl]amino]cyclopentane Carboxylic Acid Chloromethyl Ester

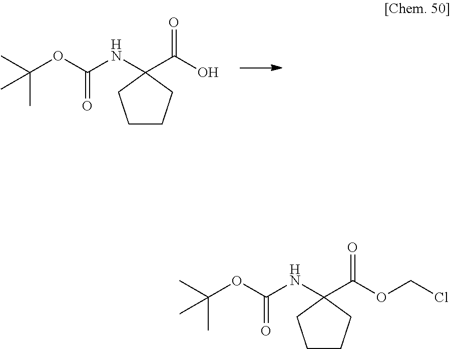 [SMILES: CC(C)(C)OC(=O)NC1(C(=O)O)CCCC1 ; CC(C)(C)OC(=O)NC1(C(=O)OCCl)CCCC1]

Under cooling on ice, a methylene chloride solution of 2.60 g (17.44 mmol) of chloromethyl chlorosulfonate was added dropwise to a water 20 ml-methylene chloride 20 ml mixed solution of 2.00 g (8.72 mmol) of 1-[[(1,1-dimethylethoxy)carbonyl]amino]cyclopentane carboxylic acid, 296 mg (0.87 mmol) of tetrabutylammonium hydrogen sulfate, and 2.93 g (34.88 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (5% to 40% ethyl acetate/hexane) to obtain 2.25 g (93%) of the title compound.

¹H-NMR (CDCl₃, δ): 1.44 (9H, s), 1.77-1.80 (4H, m), 1.88-1.90 (2H, m), 2.22-2.28 (2H, m), 4.85 (1H, br-s), 5.75 (2H, s)

### Reference Example 35

### 1-[[(1,1-dimethylethoxy)carbonyl]amino]cyclohexane Carboxylic Acid Chloromethyl Ester

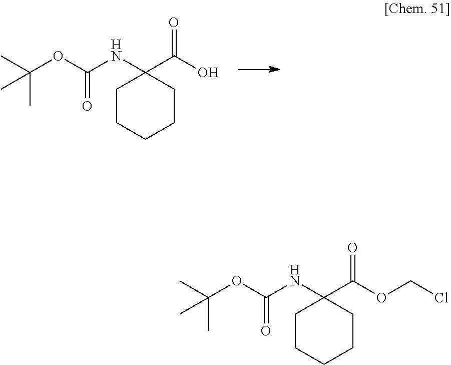 [SMILES: CC(C)(C)OC(=O)NC1(C(=O)O)CCCCC1 ; CC(C)(C)OC(=O)NC1(C(=O)OCCl)CCCCC1]

Under cooling on ice, a methylene chloride solution of 2.88 g (19.31 mmol) of chloromethyl chlorosulfonate was added dropwise to a water 20 ml-methylene chloride 20 ml mixed solution of 2.35 g (9.66 mmol) of 1-[[(1,1-dimethylethoxy)carbonyl]amino]cyclohexane carboxylic acid, 329 mg (0.97 mmol) of tetrabutylammonium hydrogen sulfate, and 23.24 g (38.64 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 2.59 g (92%) of the title compound.

¹H-NMR (CDCl₃, δ): 1.25-1.68 (15H, m), 1.86 (2H, td, J=13, 7 Hz), 1.95-1.98 (2H, m), 4.73 (1H, br-s), 5.74 (2H, s)

### Reference Example 36

### (1R,2R)-rel-2-[[(1,1-dimethylethoxy)carbonyl]amino]cyclohexane Carboxylic Acid Chloromethyl Ester

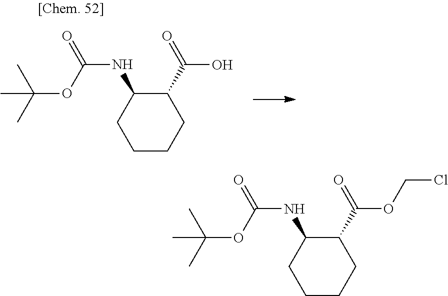 [SMILES: CC(C)(C)OC(=O)N[C@@H]1CCCC[C@H]1C(=O)O ; CC(C)(C)OC(=O)N[C@@H]1CCCC[C@H]1C(=O)OCCl]

Under cooling on ice, a methylene chloride solution of 367 mg (2.47 mmol) of chloromethyl chlorosulfonate was added dropwise to a water 5 ml-methylene chloride 6 ml mixed solution of 300 mg (1.23 mmol) of (1R,2R)-rel-2-[[(1,1-dimethylethoxy)carbonyl]amino]cyclohexane carboxylic acid, 41 mg (0.12 mmol) of tetrabutylammonium hydrogen sulfate, and 413 mg (4.92 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 311 mg (87%) of the title compound.

¹H-NMR (CDCl₃, δ):1.17-1.27 (2H, m), 1.41 (9H, s), 1.57-1.66 (2H, m), 1.72-1.77 (2H, m), 1.92-2.05 (2H, m), 2.34 (1H, td, J=12, 4 Hz), 3.65-3.70 (1H, m), 4.48 (1H, br-s), 5.69 (2H, s)

### Reference Example 37

### 2-[[(1,1-dimethylethoxy)carbonyl]amino]-2-ethylbutanoic Acid Chloromethyl Ester

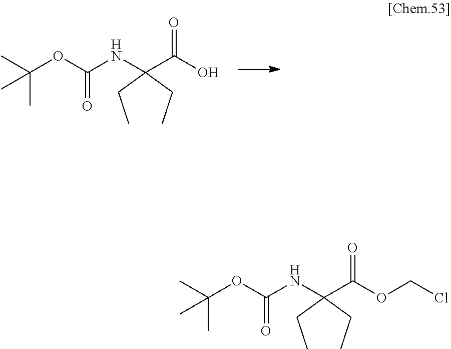 [SMILES: CCC(CC)(NC(=O)OC(C)(C)C)C(=O)O ; CCC(CC)(NC(=O)OC(C)(C)C)C(=O)OCCl]

Under cooling on ice, a methylene chloride solution of 1036 mg (6.96 mmol) of chloromethyl chlorosulfonate was added dropwise to a water 15 ml-methylene chloride 15 ml mixed solution of 804 mg (3.48 mmol) of 2-[[(1,1-dimethylethoxy)carbonyl]amino]-2-ethylbutanoic acid, 118 mg (0.35 mmol) of tetrabutylammonium hydrogen sulfate, and 1169 mg (13.92 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 882 mg (91%) of the title compound.

¹H-NMR (CDCl₃, δ):0.81 (6H, t, J=8 Hz), 1.44 (9H, s), 1.79-1.87 (2H, m), 2.22 (2H, br-s), 5.28 (1H, br-s), 5.77 (2H, s)

### Reference Example 38

### 2-[[[(1,1-dimethylethoxy)carbonyl]amino]methyl]butanoic Acid Chloromethyl Ester

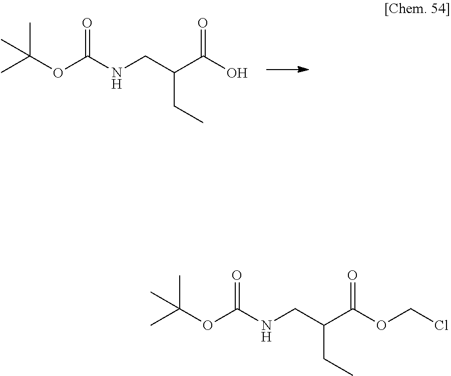 [SMILES: CCC(CNC(=O)OC(C)(C)C)C(=O)O ; CCC(CNC(=O)OC(C)(C)C)C(=O)OCCl]

Under cooling on ice, a methylene chloride solution of 2.65 g (17.77 mmol) of chloromethyl chlorosulfonate was added dropwise to a water 20 ml-methylene chloride 20 ml mixed solution of 1.93 mg (8.88 mmol) of 2-[[[(1,1-dimethylethoxy)carbonyl]amino]methyl]butanoic acid, 302 mg (0.89 mmol) of tetrabutylammonium hydrogen sulfate, and 2.98 g (35.54 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 1.97 mg (84%) of the title compound.

¹H-NMR (CDCl₃, δ):0.96 (3/2H, t, J=8 Hz), 0.97 (3/2H, t, J=8 Hz), 1.43 (9H, s), 1.57-1.74 (2H, m), 2.65 (1H, br-s), 3.27-3.31 (1H, m), 3.39-3.42 (1H, m), 4.83 (1H, br-s), 5.69-5.78 (2H, m)

### Reference Example 39

### 2-[[[(1,1-dimethylethoxy)carbonyl]amino]methyl]-3-phenylbutanoic Acid Chloromethyl Ester

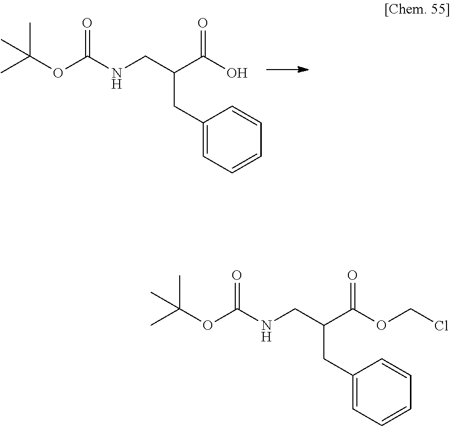 [SMILES: CC(C)(C)OC(=O)NCC(Cc1ccccc1)C(=O)O ; CC(C)(C)OC(=O)NCC(Cc1ccccc1)C(=O)OCCl]

Under cooling on ice, a methylene chloride solution of 1.86 g (12.46 mol) of chloromethyl chlorosulfonate was added dropwise to a water 20 ml-methylene chloride 20 ml mixed solution of 1.74 g (6.23 mmol) of 2-[[[(1,1-dimethylethoxy)carbonyl]amino]methyl]-3-phenylbutanoic acid, 212 mg (0.62 mmol) of tetrabutylammonium hydrogen sulfate, and 2.09 g (24.92 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 1.65 g (81%) of the title compound.

¹H-NMR (CDCl₃, δ): 1.42 (9H, s), 2.82-2.86 (1H, m), 2.96-3.03 (2H, m), 3.26-3.32 (1H, m), 3.40-3.43 (1H, m), 5.30 (1H, br-s), 5.66 (2H, s), 7.17-7.30 (5H, m)

### Reference Example 40

### 7-[[(1,1-dimethylethoxy)carbonyl]amino]heptanoic Acid Chloromethyl Ester

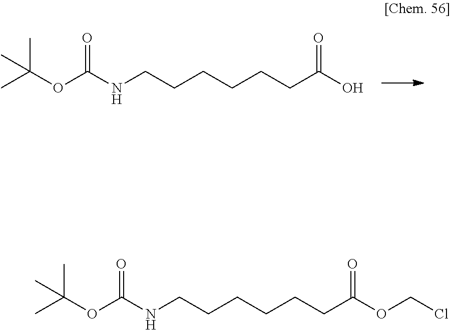 [SMILES: CC(C)(C)OC(=O)NCCCCCCC(=O)O ; CC(C)(C)OC(=O)NCCCCCCC(=O)OCCl]

Under cooling on ice, a methylene chloride solution of 1.21 g (8.15 mol) of chloromethyl chlorosulfonate was added dropwise to a water 15 ml-methylene chloride 15 ml mixed solution of 1.00 g (4.08 mmol) of 7-[[(1,1-dimethylethoxy)carbonyl]amino]heptanoic acid, 139 mg (0.41 mmol) of tetrabutylammonium hydrogen sulfate, and 1.37 g (16.32 mmol) of sodium hydrogen carbonate. The reaction solution was warmed to room temperature and stirred overnight. The methylene chloride layer of the reaction solution was isolated, washed with saturated saline, and then dried with anhydrous sodium sulfate. The solvent was condensed under reduced pressure, and the residue was purified by silica gel column chromatography (ethyl acetate/hexane) to obtain 1.11 g (92%) of the title compound.

¹H-NMR (CDCl₃, δ):1.32-1.37 (4H, m), 1.44-1.51 (1H, m), 1.63-1.67 (2H, m), 2.38 (2H, t, J=7 Hz), 3.10-3.11 (2H, m), 4.50 (1H, br-s), 5.70 (2H, s)

### Reference Example 41

### 3-[[2-[[[(1,1-dimethylethoxy)carbonyl]amino]methyl]-3-methyl-1-oxobutoxy]methyl]-2-methyl-1-[(2,3,4,9-tetrahydro-9-methyl-4-oxo-1H-carbazol-3-yl)methyl]-1H-imidazolium Chloride

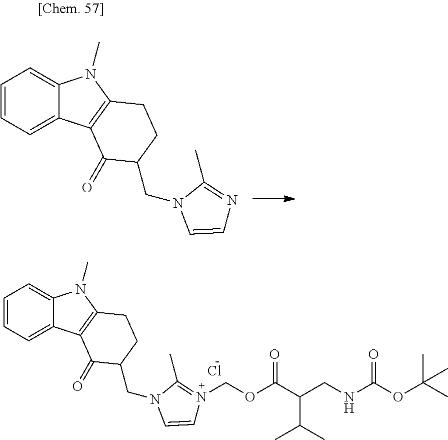

400 mg (1.36 mmol) of ondansetron was added at room temperature to an acetonitrile solution of 191 mg (0.68 mmol) of 3-methyl-2-[[[(1,1-dimethylethoxy)carbonyl]amino]methyl]butanoic acid chloromethyl ester and stirred at 100° C. overnight. The reaction solution was condensed at a water bath set to 40° C. The residue was purified by silica gel column chromatography (10→20% methanol/chloroform) to obtain 251 mg (64%) of the title compound.

¹H-NMR (CDCl₃, δ):0.87 (3/2H, d, J=8 Hz), 0.89 (3/2H, d, J=8 Hz), 0.93 (3/2H, d, J=8 Hz), 0.94 (3/2H, d, J=8 Hz), 1.36 (9/2H, s), 1.39 (9/2H, s), 1.91-2.12 (2H, m), 2.50-2.57 (1H, m), 2.77 (1H, br-s), 3.03 (3/2H, s), 3.05 (3/2H, s), 3.11-3.43 (5H, m), 3.70 (3/2H, s), 3.71 (3/2H, s), 4.55-4.61 (1H, m), 4.74 (1H, dd, J=14.7 Hz), 5.06 (1/2H, br-s), 5.10 (1/2H, br-s), 6.12-6.20 (2H, m), 7.25-7.33 (3H, m), 7.40 (1/2H, d, J=2 Hz), 7.42 (1/2H, d, J=2 Hz), 7.88-7.90 (1H, m), 8.07-8.10 (1H, m)

### Example 1

### 3-[[2-(aminomethyl)-3-methyl-1-oxobutoxy]methyl]-2-methyl-1-[(2,3,4,9-tetrahydro-9-methyl-4-oxo-1H-carbazol-3-yl)methyl]-1H-imidazolium Chloride Hydrochloride

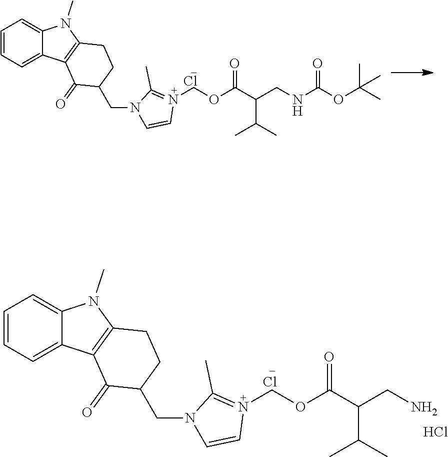

8 ml of 4 N hydrochloric acid/dioxane solution was added under cooling on ice to 8 ml of chloroform solution of 213 mg (0.37 mmol) of 3-[[2-[[[(1,1-dimethylethoxy)carbonyl]amino]methyl]-3-methyl-1-oxobutoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 2 hours. Thereafter, the solvent was distilled off under reduced pressure. Ethyl acetate was added to the residue and stirred for 1 hour. Crystals were filtered to obtain 214 mg (quantitative) of the title compound.

¹H-NMR (DMSO-d₆, δ):0.82-0.84 (3H, m), 0.87-0.88 (3H, m), 1.99-2.18 (3H, m), 2.72 (1H, br-s), 2.81 (3/2H, s), 2.82 (3/2H, s), 2.94-3.19 (5H, m), 3.75 (3/2H, s), 3.76 (3/2H, s), 4.35-4.37 (1H, m), 4.72-4.76 (1H, m), 6.20 (2H, br-s), 7.22 (1H, td, J=8, 3 Hz), 7.27 (1H, td, J=8, 3 Hz), 7.57 (1H, dd, J=8, 3 Hz), 7.81 (1H, d, J=2 Hz), 7.88 (1H, d, J=2 Hz), 7.98 (1H, dd, J=8.3 Hz), 8.28 (3H, br-s)

### Example 2

### [3-methyl-2-[[(ondansetron)methoxy]carbonyl]butyl]amino-chondroitin Sulfate Conjugate

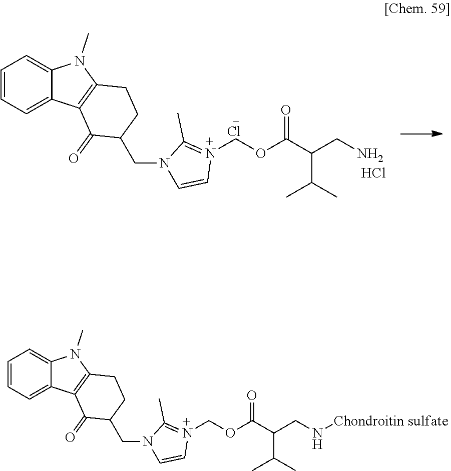

1 ml of ethanol was slowly added dropwise under stirring to 4.0 g (0.398 mmol) of 5% chondroitin sulfate aqueous solution (prepared by dissolving sodium chondroitin sulfate). To the mixed liquid, a mixed solution of 41 mg (0.080 mmol) of 3-[[2-(aminomethyl)-3-methyl-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 3 ml of ethanol and 1 ml of water was added and then a mixed solution of 38 mg (0.08 mmol) of 4-(4,6-dimethoxy-1,3,5-triazin-2-yl)-4-methylmorpholinium chloride (DMT-MM) in 2 ml of ethanol and 1 ml of water was added, and the resultant liquid was stirred at room temperature overnight. 100 μl of 20% sodium chloride aqueous solution was added to the reaction solution, and ethanol was further added dropwise (3 ml) until immediately before the reaction solution became cloudy. The reaction solution was added dropwise under stirring to 8 ml of 90% ethanol, and 11 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 216 mg of the title compound. Based on values of integral in ¹H-NMR, the introduction rate of ondansetron per unit of whole disaccharide (glucuronic acid) of chondroitin sulfate was 23%.

### Reference Example 42

### 3-[[2-[[(1,1-dimethylethoxy)carbonyl]amino]-1-oxopropoxy]methyl]-2-methyl-1-[(2,3,4,9-tetrahydro-9-methyl-4-oxo-1H-carbazol-3-yl)methyl]-1H-imidazolium Chloride

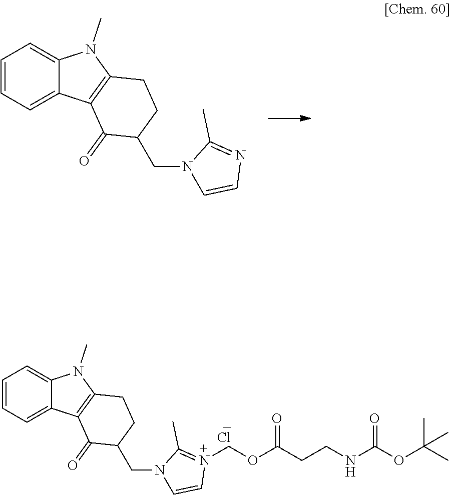

400 mg (1.36 mmol) of ondansetron was added at room temperature to an acetonitrile solution of 164 mg (0.68 mmol) of N-[(1,1-dimethylethoxy)carbonyl]-β-alanine chloromethyl ester and stirred at 100° C. overnight. The reaction solution was condensed at a water bath set to 40° C. The residue was purified by silica gel column chromatography (12→20% methanol/chloroform) to obtain 230 mg (64%) of the title compound.

¹H-NMR (CDCl₃, δ):1.37 (9/2H, s), 1.40 (9/2H, s), 2.05 (1H, br-s), 2.60-2.64 (2H, m), 2.76 (1H, br-s), 3.02 (3/211H, s), 3.04 (3/2H, s), 3.10-3.38 (5H, m), 3.67 (3/2H, s), 3.70 (3/2H, s), 4.53-4.59 (1H, m), 4.72-4.77 (1H, m), 5.08 (1H, br-s), 6.14-6.19 (2H, m), 7.23-7.33 (3H, m), 7.40 (1/2H, br-s), 7.42 (1/2H, br-s), 7.83 (1/2H, br-s), 7.85 (1/2H, br-s), 8.08-8.10 (1H, m)

### Example 3

### 3-[(2-amino-1-oxopropoxy)methyl]-2-methyl-1-[(2,3,4,9-tetrahydro-9-methyl-4-oxo-1H-carbazol-3-yl)methyl]-1H-imidazolium Chloride Hydrochloride

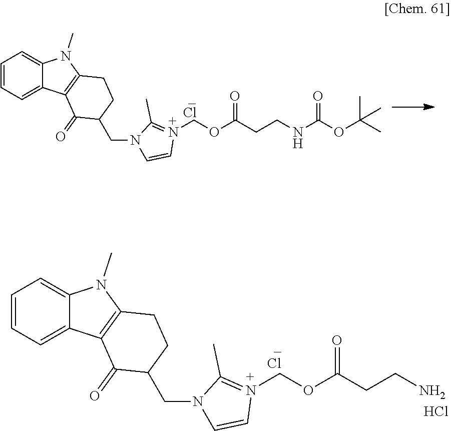

3 ml of 4 N hydrochloric acid/dioxane solution was added under cooling on ice to 3 ml of chloroform solution of 230 mg (0.43 mmol) of 3-[[2-[[(1,1-dimethylethoxy)carbonyl]amino]-1-oxopropoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 2 hours. Thereafter, the solvent was distilled off under reduced pressure. Ethyl acetate was added to the residue and stirred for 2.5 hours. Crystals were filtered to obtain 127 mg (63%) of the title compound.

¹H-NMR (DMSO-d₆, δ):1.95-2.02 (1H, m), 2.18-2.21 (1H, m), 2.79 (3H, s), 2.84 (2H, t, J=7 Hz), 2.98-3.19 (5H, m), 3.75 (3H, s), 4.35 (1H, dd, J=15, 7 Hz), 4.72 (1H, dd, J=15, 6 Hz), 6.15 (2H, s), 7.22 (1H, t, J=8 Hz), 7.27 (1H, t, J=8 Hz), 7.57 (1H, d, J=8 Hz), 7.80 (1H, d, J=2 Hz), 7.85 (1H, d, J=2 Hz), 7.98 (1H, d, J=8 Hz), 8.33 (3H, br-s)

### Example 4

### [3-[(ondansetron)methoxy]-3-oxopropyl]amino-chondroitin Sulfate Conjugate n

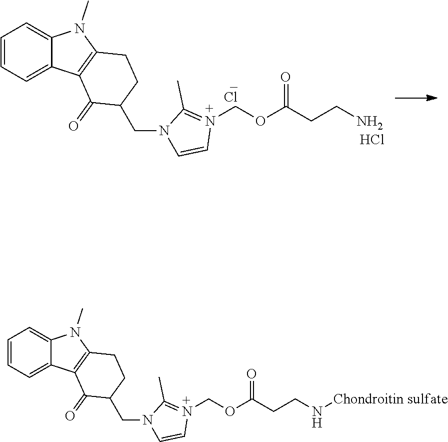

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 37 mg (0.080 mmol) of 3-[(2-amino-1-oxopropoxy)methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 217 mg of the title compound. Based on values of integral in ¹H-NMR, the introduction rate of ondansetron per unit of whole disaccharide (glucuronic acid) of chondroitin sulfate was 20%.

### Reference Example 43

### 3-[[2-[[[(1,1-dimethylethoxy)carbonyl]amino]methyl]-2-ethyl-1-oxobutoxy]methyl]-2-30 methyl-1-[(2,3,4,9-tetrahydro-9-methyl-4-oxo-1H-carbazol-3-yl)methyl]-1H-imidazolium Chloride

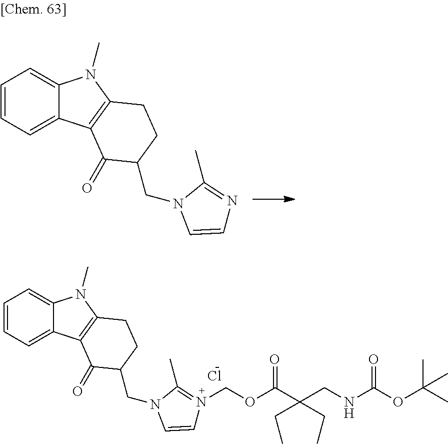

379 mg (1.29 mmol) of ondansetron was added at room temperature to an acetonitrile solution of 190 mg (0.65 mmol) of 2-ethyl-2-[[[(1,1-dimethylethoxy)carbonyl]amino]methyl]butanoic acid chloromethyl ester and stirred at 100° C. overnight. The reaction solution was condensed at a water bath set to 40° C., and then the residue was purified by silica gel column chromatography (15→20% methanol/chloroform) to obtain 297 mg (78%) of the title compound.

¹H-NMR (CDCl₃, δ):0.77 (6H, t, J=8 Hz), 1.38 (9H, s), 1.55-1.65 (4H, m), 2.05 (1H, qd, J=12, 5 Hz), 2.80-2.82 (1H, m), 3.06 (3H, s), 3.13-3.37 (5H, m), 3.70 (3H, s), 4.57 (1H, dd, J=14, 5 Hz), 4.67 (1H, br-s), 4.76 (1H, dd, J=14, 7 Hz), 6.12 (1H, d, J=11 Hz), 6.18 (1H, d, J=11 Hz), 7.25-7.33 (3H, m), 7.36 (1H, d, J=2 Hz), 7.87 (1H, d, J=2 Hz), 8.09-8.11 (1H, m)

### Example 5

### 3-[[2-(aminomethyl)-2-ethyl-1-oxobutoxy]methyl]-2-methyl-1-[(2,3,4,9-tetrahydro-9-m ethyl-4-oxo-1H-carbazol-3-yl)methyl]-1H-imidazolium Chloride Hydrochloride

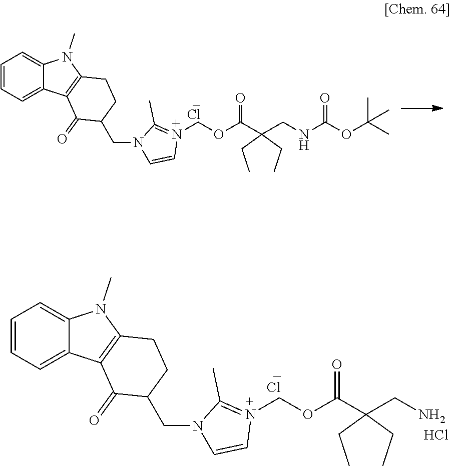

2 ml of 4 N hydrochloric acid/dioxane solution was added to 2 ml of chloroform solution of 295 mg (0.50 mmol) of 3-[[2-[[[(1,1-dimethylethoxy)carbonyl]amino]methyl]-2-ethyl-1-oxobutoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 220 mg (84%) of the title compound.

¹H-NMR (DMSO-d₆, δ): 0.71 (6H, t, J=8 Hz), 1.65 (4H, q, J=8 Hz), 1.97 (1H, qd, J=12, 5 Hz), 2.14-2.18 (1H, m), 2.81 (3H, s), 2.98-3.20 (5H, m), 3.75 (3H, s), 4.37 (1H, dd, J=14, 7 Hz), 4.73 (1H, dd, J=14, 7 Hz), 6.18 (1H, d, J=11 Hz), 6.21 (1H, d, J=1 Hz), 7.22 (1H, td, J=8, 1 Hz), 7.27 (1H, td, J=8, 1 Hz), 7.56 (1H, d, J=8 Hz), 7.81 (1H, d, J=3 Hz), 7.87 (1H, d, J=3 Hz), 7.97 (1H, d, J=8 Hz), 8.28 (3H, br-s)

### Example 6

### [2-ethyl-2-[[(ondansetron)methoxy]carbonyl]butyl]amino-chondroitin Sulfate Conjugate

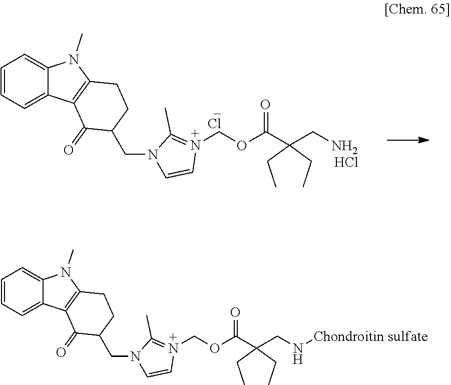

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[[2-(aminomethyl)-2-ethyl-1-oxobutoxy]methyl]-2-methyl-1-[(2,3,4,9-tetrahydro-9-m ethyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 217 mg of the title compound. Based on values of integral in ¹H-NMR, the introduction rate of ondansetron per unit of whole disaccharide (glucuronic acid) of chondroitin sulfate was 21%.

### Reference Example 44

### 3-[[[[1-[[[(1,1-dimethylethoxy)carbonyl]amino]methyl]cyclopropyl]carbonyl]oxy]meth yl]-2-methyl-1-[(2,3,4,9-tetrahydro-9-methyl-4-oxo-1H-carbazol-3-yl)methyl]-1H-imidazolium Chloride

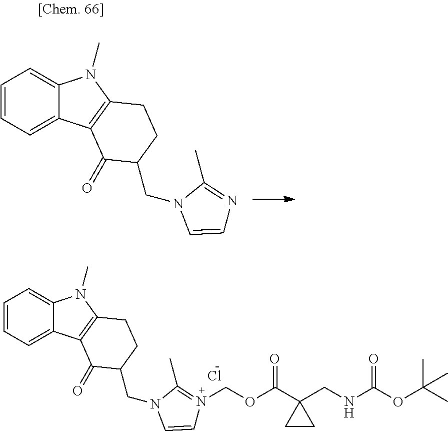

779 mg (2.66 mmol) of ondansetron was added at room temperature to an acetonitrile solution of 350 mg (1.33 mmol) of 1-[[[(1,1-dimethylethoxy)carbonyl]amino]methyl]cyclopropanecarboxylic acid chloromethyl ester and stirred at 100° C. overnight. The reaction solution was condensed at a water bath set to 40° C., and then the residue was purified by silica gel column chromatography (15% methanol/chloroform) to obtain 487 mg (66%) of the title compound.

¹H-NMR (CDCl₃, δ):1.11 (2H, br-s), 1.30 (2H, br-s), 1.42 (9H, s), 2.06 (1H, qd, J=12, 6 Hz), 2.76-2.77 (1H, m), 3.04 (3H, s), 3.12-3.32 (5H, m), 3.70 (311, s), 4.58 (1H, dd, J=14, 5 Hz), 4.77 (1H, dd, J=14, 7 Hz), 5.16 (1H, br-s), 6.13 (1H, d, J=12 Hz), 6.19 (1H, d, J=12 Hz), 7.26-7.33 (3H, m), 7.42 (1H, br-s), 7.87 (1H, br-s), 8.11 (1H, d, J=7 Hz)

### Example 7

### 3-[[[[1-(aminomethyl)cyclopropyl]carbonyl]oxy]methyl]-2-methyl-1-[(2,3,4,9-tetrahydro-9-methyl-4-oxo-1H-carbazol-3-yl)methyl]-1H-imidazolium Chloride Hydrochloride

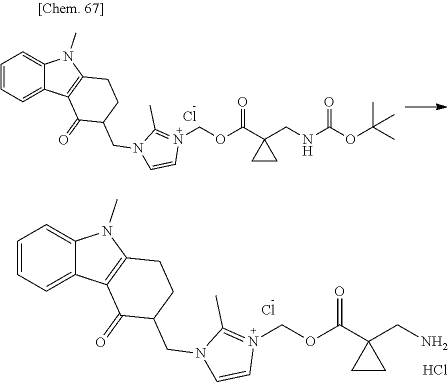

3 ml of 4 N hydrochloric acid/dioxane solution was added to 3 ml of chloroform solution of 455 mg (0.82 mmol) of 3-[[[[1-[[[(1,1-dimethylethoxy)carbonyl]amino]methyl]cyclopropyl]carbonyl]oxy]meth yl]-2-methyl-l-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Diethyl ether was added to the residue and stirred for 1 hour. Crystals were filtered to obtain 337 mg (83%) of the title compound.

¹H-NMR (DMSO-d₆, δ):1.28 (4H, s), 1.99 (1H, qd, J=13, 5 Hz), 2.18-2.21 (1H, m), 2.78 (3H, s), 2.98-3.20 (5H, m), 3.75 (3H, s), 4.35 (1H, dd, J=15, 7 Hz), 4.72 (1H, dd, J=15, 7 Hz), 6.11 (2H, s), 7.22 (1H, t, J=8 Hz), 7.27 (1H, t, J=8 Hz), 7.56 (1H, d, J=8 Hz), 7.78 (1H, d, J=2 Hz), 7.82 (1H, d, J=2 Hz), 7.99 (1H, d, J=8 Hz), 8.22 (3H, br-s)

### Example 8

### [[1-[[(ondansetron)methoxy]carbonyl]cyclopropyl]methyl]amino-chondroitin Sulfate Conjugate

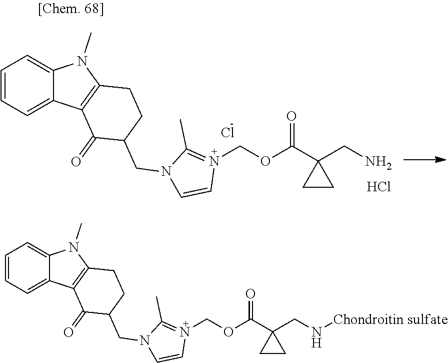

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 40 mg (0.080 mmol) of 3-[[[[1-(aminomethyl)cyclopropyl]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1.5 ml of ethanol was added, a solution of 38 mg (0.08 mmol) of 4-(4,6-dimethoxy-1,3,5-triazin-2-yl)-4-methylmorpholinium chloride (DMT-MM) in 0.5 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 217 mg of the title compound. Based on values of integral in ¹H-NMR, the introduction rate of ondansetron per unit of whole disaccharide (glucuronic acid) of chondroitin sulfate was 19%.

### Reference Example 45

### 3-[[2-[[[(1,1-dimethylethoxy)carbonyl]amino]methyl]-3,3-dimethyl-1-oxobutoxy]methy 1]-2-methyl-1-[(2,3,4,9-tetrahydro-9-methyl-4-oxo-1H-carbazol-3-yl)methyl]-1H-imidazolium Chloride

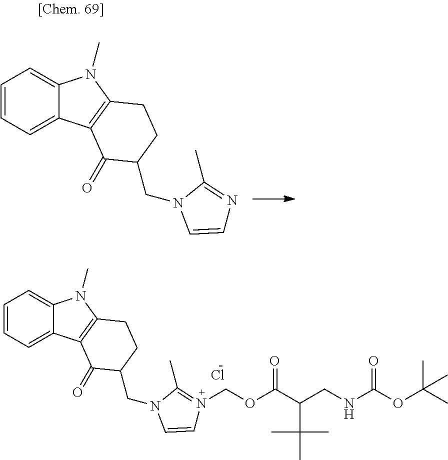

360 mg (1.22 mmol) of ondansetron was added at room temperature to an acetonitrile solution of 180 mg (0.61 mmol) of 2-[[[(1,1-dimethylethoxy)carbonyl]amino]methyl]-3,3-dimethyl-butanoic acid chloromethyl ester and stirred at 100° C. overnight. The reaction solution was condensed at a water bath set to 40° C., and then the residue was purified by silica gel column chromatography (15% methanol/chloroform) to obtain 233 mg (69%) of the title compound.

¹H-NMR (CDCl₃, δ):0.94 (9/2H, s), 0.96 (9/2H, s), 1.35 (9/2H, s), 1.39 (9/2H, s), 2.02-2.17 (1H, m), 2.60-2.67 (1H, m), 2.75-2.78 (1H, m), 3.01 (3/2H, s), 3.05 (3/2H, s), 3.11-3.48 (5H, m), 3.70 (3/2H, s), 3.72 (3/2H, s), 4.56-4.63 (1H, m), 4.72 (1H, dd, J=15, 8 Hz), 5.05 (1/2H, br-s), 5.15 (1/2H, br-s), 6.13-6.16 (2H, m), 7.24-7.33 (3H, m), 7.37 (1/2H, d, J=2 Hz), 7.39 (1/2H, d, J=2 Hz), 7.85 (1/2H, d, J=2 Hz), 7.90 (1/2H, br-s), 8.06-8.09 (1H, m)

### Example 9

### 3-[[2-(aminomethyl)-3,3-dimethyl-1-oxobutoxy]methyl]-2-methyl-1-[(2,3,4,9-tetrahydro-9-methyl-4-oxo-1H-carbazol-3-yl)methyl]-1H-imidazolium Chloride Hydrochloride

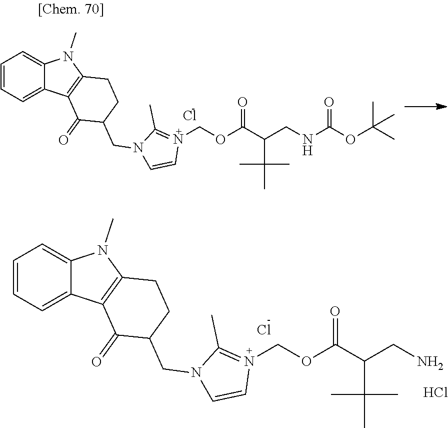

2 ml of 4 N hydrochloric acid/dioxane solution was added to 2 ml of chloroform solution of 233 mg (0.42 mmol) of 3-[[2-[[[(1,1-dimethylethoxy)carbonyl]amino]methyl]-3,3-dimethyl-1-oxobutoxy]methy 1]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 3 hours. Crystals were filtered to obtain 180 mg (82%) of the title compound.

¹H-NMR (DMSO-d₆, δ):0.88 (9/2H, s), 0.89 (9/2H, s), 1.97 (1H, qd, J=12, 5 Hz), 2.13-2.16 (1H, m), 2.49-2.57 (1H, m), 2.81 (3/2H, s), 2.82 (3/2H, s), 2.97-3.19 (5H, m), 3.74 (3H, s), 4.36 (1/2H, dd, J=15, 7 Hz), 4.37 (1/2H, dd, J=15, 7 Hz), 4.73 (1H, dd, J=15, 7 Hz), 6.14 (1H, d, J=11 Hz), 6.22 (1/2H, d, J=11 Hz), 6.23 (1/2H, d, J=1 Hz), 7.20-7.28 (2H, m), 7.56 (1H, d, J=8 Hz), 7.80-7.81 (1H, m), 7.87-7.88 (1H, m), 7.98 (1H, d, J=8 Hz), 8.19 (3H, br-s)

### Example 10

### [3,3-dimethyl-2-[[(ondansetron)methoxy]carbonyl]butyl]amino-chondroitin Sulfate Conjugate

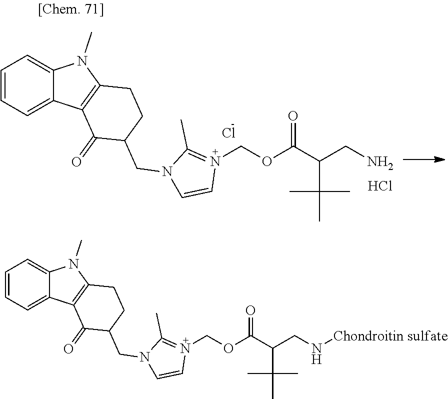

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[[2-(aminomethyl)-3,3-dimethyl-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 1.5 ml of ethanol was added, a solution of 38 mg (0.08 mmol) of 4-(4,6-dimethoxy-1,3,5-triazin-2-yl)-4-methylmorpholinium chloride (DMT-MM) in 0.5 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 217 mg of the title compound. Based on values of integral in ¹H-NMR, the introduction rate of ondansetron per unit of whole disaccharide (glucuronic acid) of chondroitin sulfate was 22%.

### Reference Example 46

3-[[[[1-[[(1,1-dimethylethoxy)carbonyl]amino]cyclopentyl]carbonyl]oxy]methyl]-2-methyl-1-[(2,3,4,9-tetrahydro-9-methyl-4-oxo-1H-carbazol-3-yl)methyl]-1H-imidazolium Chloride

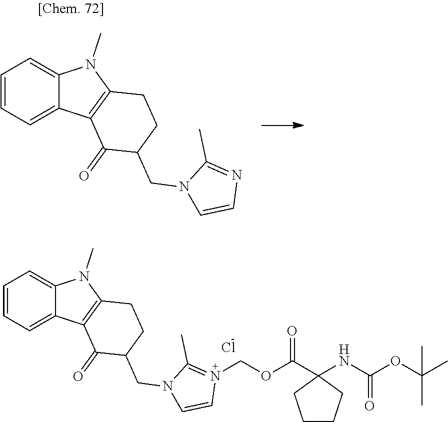

461 mg (1.57 mmol) of ondansetron was added at room temperature to an acetonitrile solution of 218 mg (0.78 mmol) of 1-[[(1,1-dimethylethoxy)carbonyl]amino]cyclopentane carboxylic acid chloromethyl ester and stirred at 100° C. overnight. The reaction solution was condensed at a water bath set to 40° C., and then the residue was purified by silica gel column chromatography (15% methanol/chloroform) to obtain 226 mg (51%) of the title compound.

¹H-NMR (CDCl₃, δ): 1.34 (9H, s), 1.76-1.87 (6H, m), 1.99-2.22 (3H, m), 2.73-2.75 (1H, m), 3.04 (311, s), 3.08-3.34 (3H, m), 3.70 (3H, s), 4.49 (1H, dd, J=14, 6 Hz), 4.75 (1H, dd, J=14, 6 Hz), 4.91 (1H, br-s), 6.19 (1H, d, J=12 Hz), 6.26 (1H, d, J=12 Hz), 7.26-7.32 (3H, m), 7.41 (1H, d, J=3 Hz), 7.77 (1H, br-s), 8.11-8.12 (1H, m)

### Example 11

### 3-[[[(1-aminocyclopentyl)carbonyl]oxy]methyl]-2-methyl-1-[(2,3,4,9-tetrahydro-9-meth yl-4-oxo-1H-carbazol-3-yl)methyl]-1H-imidazolium Chloride Hydrochloride

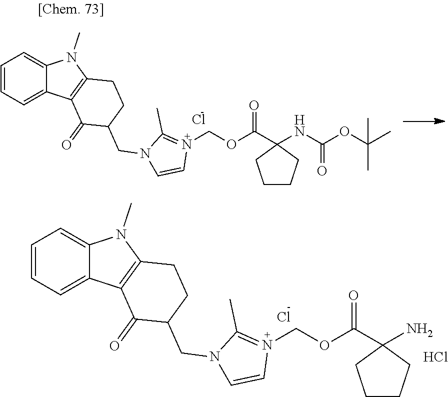

2 ml of 4 N hydrochloric acid/dioxane solution was added to 2 ml of chloroform solution of 210 mg (0.37 mmol) of 3-[[[[1-[[(1,1-dimethylethoxy)carbonyl]amino]cyclopentyl]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 185 mg (99%) of the title compound.

¹H-NMR (DMSO-d₆, δ):1.74-2.20 (10H, m), 2.80 (3H, s), 3.00-3.19 (3H, m), 3.75 (3H, s), 4.36 (1H, dd, J=14, 7 Hz), 4.74 (1H, dd, J=14, 7 Hz), 6.25 (1H, d, J=10 Hz), 6.29 (1H, d, J=10 Hz), 7.22 (1H, t, J=8 Hz), 7.27 (1H, t, J=8 Hz), 7.56 (1H, d, J=8 Hz), 7.80 (1H, s), 7.85 (1H, s), 7.97 (1H, d, J=8 Hz), 8.78 (3H, br-s)

### Example 12

### [1-[[(ondansetron)methoxy]carbonyl]cyclopentyl]amino-chondroitin Sulfate Conjugate

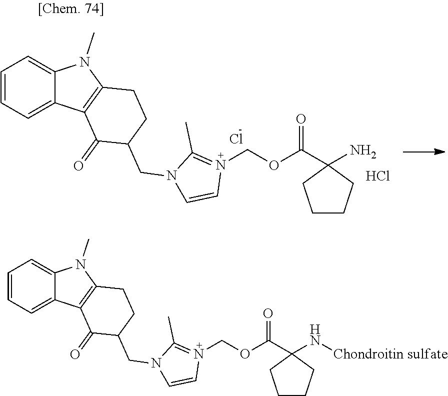

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 41 mg (0.080 mmol) of 3-[[[(1-aminocyclopentyl)carbonyl]oxy]methyl]-2-methyl-1-[(2,3,4,9-tetrahydro-9-meth yl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 185 mg of the title compound. Based on values of integral in ¹H-NMR, the introduction rate of ondansetron per unit of whole disaccharide (glucuronic acid) of chondroitin sulfate was 0.4%.

### Reference Example 47

### 3-[[[[(1R,2R)-rel-2-[[(1,1-dimethylethoxy)carbonyl]amino]cyclohexyl]carbonyl]oxy]m ethyl]-2-methyl-1-[(2,3,4,9-tetrahydro-9-methyl-4-oxo-1H-carbazol-3-yl)methyl]-1H-imidazolium Chloride

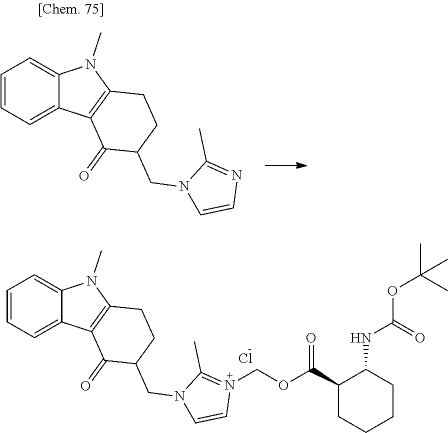

251 mg (0.86 mmol) of ondansetron was added at room temperature to an acetonitrile solution of 125 mg (0.43 mmol) of (1R,2R)-rel-2-[[(1,1-dimethylethoxy)carbonyl]amino]cyclohexane carboxylic acid chloromethyl ester and stirred at 100° C. overnight. The reaction solution was condensed at a water bath set to 40° C., and then the residue was purified by silica gel column chromatography (15% methanol/chloroform) to obtain 114 mg (45%) of the title compound.

¹H-NMR (CDCl₃, δ):1.14-1.27 (4H, m), 1.37 (9H, s), 1.50-2.01 (4H, m), 2.02-2.11 (1H, m), 2.33-2.35 (1H, m), 2.76-2.79 (1H, m), 3.02 (3/2H, s), 3.03 (3/2H, s), 3.12-3.34 (3H, m), 3.64 (1H, br-s), 3.70(3/2H, s), 3.71(3/2H, s), 4.55-4.68 (2H, m), 4.75(1/2H, dd, J=14, 7 Hz), 4.76(1/2H, dd, J=14, 7 Hz), 6.04-6.12 (2H, m), 7.24-7.32 (3H, m), 7.41(1/2H, d, J=3 Hz), 7.42(1/2H, d, J=3 Hz), 7.83-7.90 (1H, m), 8.08-8.10(1H, m)

### Example 13

### 3-[[[[(1R,2R)-rel-2-aminocyclohexyl]carbonyl]oxy]methyl]-2-methyl-1-[(2,3,4,9-tetrahydro-9-methyl-4-oxo-1H-carbazol-3-yl)methyl]-1H-imidazolium Chloride Hydrochloride

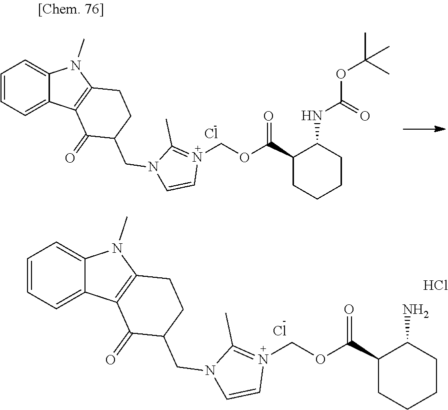

2 ml of 4 N hydrochloric acid/dioxane solution was added to 2 ml of chloroform solution of 114 mg (0.19 mmol) of 3-[[[[(1R,2R)-rel-2-[[(1,1-dimethylethoxy)carbonyl]amino]cyclohexyl]carbonyl]oxy]m ethyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 2 hours. Thereafter, the solvent was distilled off under reduced pressure. Ethyl acetate was added to the residue and stirred for 1 hour. Crystals were filtered to obtain 86 mg (87%) of the title compound.

¹H-NMR (DMSO-d₆, δ):1.15-1.42 (4H, m), 1.64-1.73 (2H, m), 1.95-2.03 (3H, m), 2.17-2.20 (1H, m), 2.61-2.66(1H, m), 2.80 (3H, s), 2.99-3.25 (4H, m), 3.75 (3H, s), 4.36 (1H, dd, J=14, 7 Hz), 4.72 (1H, dd, J=14, 7 Hz), 6.16 (1H, d, J=13 Hz), 6.19 (1H, d, J=13 Hz), 7.22 (1H, t, J=8 Hz), 7.25-7.28 (1H, m), 7.56 (1H, d, J=8 Hz), 7.80 (1H, d, J=2 Hz), 7.87(1/2H, d, J=2 Hz), 7.88(1/2H, d, J=2 Hz), 7.98(1H, d, J=8 Hz), 8.36(3H, br-s)

### Example 14

### [2-[[(ondansetron)methoxy]carbonyl]-(1R,2R)-rel-cyclohexyl]amino-chondroitin Sulfate Conjugate

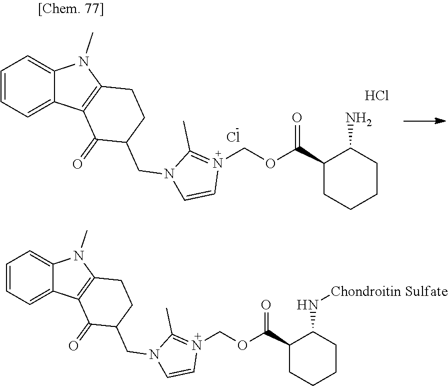

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 41 mg (0.080 mmol) of 3-[[[[(1R,2R)-rel-2-aminocyclohexyl]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 193 mg of the title compound. Based on values of integral in ¹H-NMR, the introduction rate of ondansetron per unit of whole disaccharide (glucuronic acid) of chondroitin sulfate was 6%.

### Reference Example 48

### 3-[[[[1-[[(1,1-dimethylethoxy)carbonyl]amino]cyclohexyl]carbonyl]oxy]methyl]-2-methyl-1-[(2,3,4,9-tetrahydro-9-methyl-4-oxo-1H-carbazol-3-yl)methyl]-1H-imidazolium Chloride

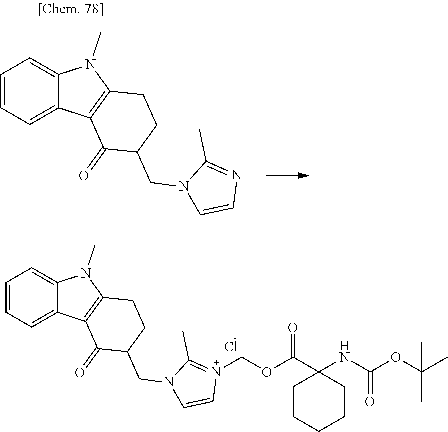

481 mg (1.64 mmol) of ondansetron was added at room temperature to an acetonitrile solution of 238 mg (0.82 mmol) of 1-[[(1,1-dimethylethoxy)carbonyl]amino]cyclohexane carboxylic acid chloromethyl ester and stirred at 100° C. overnight. The reaction solution was condensed at a water bath set to 40° C., and then the residue was purified by silica gel column chromatography (15% methanol/chloroform) to obtain 115 mg (24%) of the title compound.

¹H-NMR (CDCl₃, δ):1.32 (9H, s), 1.44-1.88 (10H, m), 1.98-2.08(1H, m), 2.73-2.75 (1H, m), 3.04 (3H, s), 3.10-3.34 (3H, m), 3.70 (3H, s), 4.48 (1H, dd, J=14, 6 Hz), 4.75 (1H, dd, J=14, 6 Hz), 4.85(1H, br-s), 6.19 (1H, d, J=11 Hz), 6.25(1H, d, J=11 Hz), 7.25-7.33 (3H, m), 7.40 (1H, d, J=2 Hz), 7.75(1H, br-s), 8.11-8.12 (1H, m)

### Example 15

### 3-[[[(1-aminocyclohexyl)carbonyl]oxy]methyl]-2-methyl-1-[(2,3,4,9-tetrahydro-9-meth yl-4-oxo-1H-carbazol-3-yl)methyl]-1H-imidazolium Chloride Hydrochloride

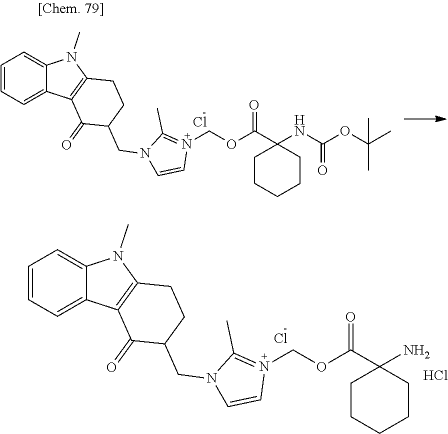

2 ml of 4 N hydrochloric acid/dioxane solution was added to 2 ml of chloroform solution of 115 mg (0.20 mmol) of 3-[[[[1-[[(1,1-dimethylethoxy)carbonyl]amino]cyclohexyl]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 95 mg (93%) of the title compound.

¹H-NMR (DMSO-d₆, δ):1.38-1.79 (6H, m), 1.96-2.00 (5H, m), 2.19-2.22 (1H, m), 2.82 (3H, s), 2.99-3.19 (3H, m), 3.75 (3H, s), 4.34(1H, dd, J=14, 7 Hz), 4.75 (1H, dd, J=14, 7 Hz), 6.25(1H, d, J=1 Hz), 6.29 (1H, d, J=1 Hz), 7.22 (1H, t, J=8 Hz), 7.27(1H, t, J=8 HZ), 7.56 (1H, d, J=8 Hz), 7.79 (1H, d, J=2 Hz), 7.85 (1H, d, J=2 Hz), 7.98 (1H, d, J=8 Hz), 8.36(3H, br-s)

### Example 16

### [1-[[(ondansetron)methoxy]carbonyl]cyclohexyl]amino-chondroitin Sulfate Conjugate

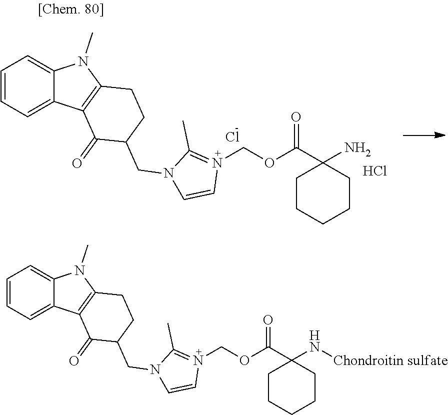

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[[[(1-aminocyclohexyl)carbonyl]oxy]methyl]-2-methyl-1-[(2,3,4,9-tetrahydro-9-meth yl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 0.5 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 177 mg of the title compound. Based on values of integral in ¹H-NMR, the introduction rate of ondansetron per unit of whole disaccharide (glucuronic acid) of chondroitin sulfate was 0.5%.

### Reference Example 49

### 3-[([[1-[[[(1,1-dimethylethoxy)carbonyl]amino]methyl]cyclopentyl]carbonyl]oxy]methyl]-2-methyl-1-[(2,3,4,9-tetrahydro-9-methyl-4-oxo-1H-carbazol-3-yl)methyl]-1H-imidazolium Chloride

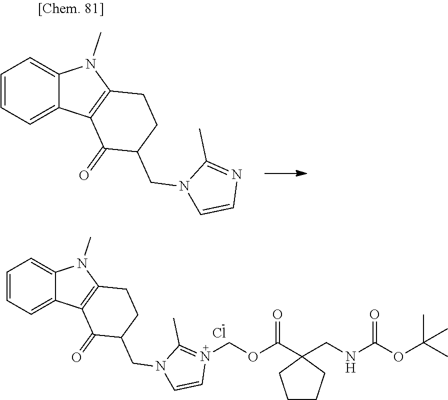

500 mg (1.70 mmol) of ondansetron was added at room temperature to an acetonitrile solution of 249 mg (0.85 mmol) of 1-[[[(1,1-dimethylethoxy)carbonyl]amino]methyl]cyclopentane carboxylic acid chloromethyl ester and stirred at 100° C. overnight. The reaction solution was condensed at a water bath set to 40° C., and then residue was purified by silica gel column chromatography (15% methanol/chloroform) to obtain 408 mg (82%) of the title compound.

¹H-NMR (CDCl₃, δ):1.38 (9H, s), 1.60-1.97 (8H, m), 2.06(1H, qd, J=12, 5 Hz), 2.76-2.78 (1H, m), 3.03 (3H, s), 3.12-3.34 (5H, m), 3.70 (3H, s), 4.57 (1H, dd, J=14, 5 Hz), 4.75(1H, dd, J=14, 7 Hz), 5.03(1H, br-s), 6.14(1H, d, J=1 Hz), 6.18(1H, d, J=11 Hz), 7.25-7.33 (3H, m), 7.43(1H, br-s), 7.88(1H, br-s), 8.09 (1H, d, J=7 Hz)

### Example 17

### 3-[[[[1-(aminomethyl)cyclopentyl]carbonyl]oxy]methyl]-2-methyl-1-[(2,3,4,9-tetrahydro-9-methyl-4-oxo-1H-carbazol-3-yl)methyl]-1H-imidazolium Chloride Hydrochloride

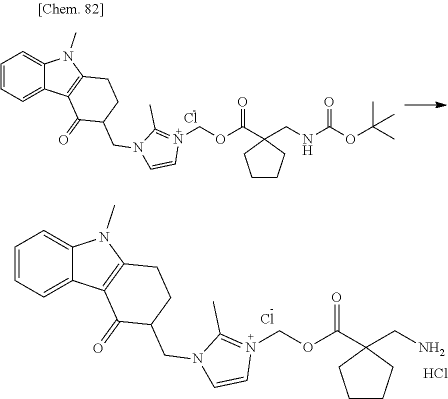

2 ml of 4 N hydrochloric acid/dioxane solution was added to 2 ml of chloroform solution of 408 mg (0.70 mmol) of 3-[[[[1-[[[(1,1-dimethylethoxy)carbonyl]amino]methyl]cyclopentyl]carbonyl]oxy]meth yl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Acetic acid ester was added to the residue and stirred for 1 hour. Crystals were filtered to obtain 294 mg (80%) of the title compound.

¹H-NMR (DMSO-d₆, δ):1.65-1.72 (6H, m), 1.95-2.03 (3H, m), 2.16-2.20 (1H, m), 2.78 (3H, s), 2.98-3.04 (3H, m), 3.12-3.20 (2H, m), 3.75 (3H, s), 4.36(1H, dd, J=14, 7 Hz), 4.72 (1H, dd, J=14, 7 Hz), 6.13(1H, d, J=11), 6.15(1H, d, J=1), 7.20-7.23 (1H, m), 7.27(1H, td, J=8, 2 Hz), 7.56 (1H, d, J=8 Hz), 7.79 (1H, d, J=2 Hz), 7.85 (1H, d, J=2 Hz), 7.97 (1H, d, J=8 Hz), 8.22(3H, br-s)

### Example 18

### [[1-[[(ondansetron)methoxy]carbonyl]cyclopentyl]methyl]amino-chondroitin Sulfate Conjugate

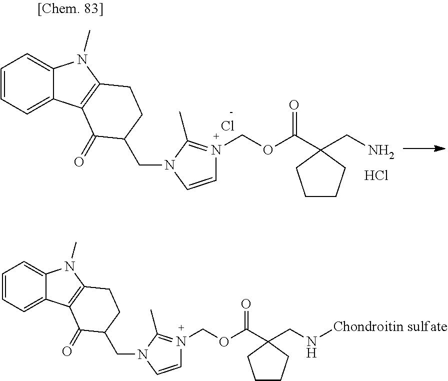

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[[[[1-(aminomethyl)cyclopentyl]carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 188 mg of the title compound. Based on values of integral in ¹H-NMR, the introduction rate of ondansetron per unit of whole disaccharide (glucuronic acid) of chondroitin sulfate was 21%.

### Reference Example 50

### 3-[[3-[[(1,1-dimethylethoxy)carbonyl]amino]-2-methyl-1-oxopropoxy]methyl]-2-methy 1-1-[(2,3,4,9-tetrahydro-9-methyl-4-oxo-1H-carbazol-3-yl)methyl]-1H-imidazolium Chloride

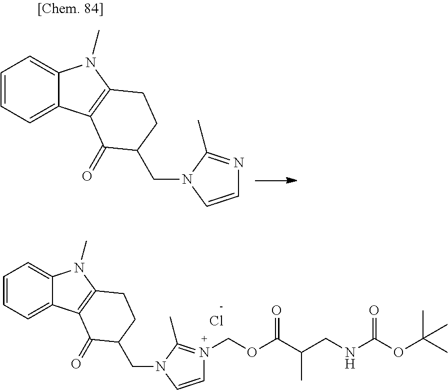

396 mg (1.35 mmol) of ondansetron was added at room temperature to an acetonitrile solution of 170 mg (0.68 mmol) of 3-[[[(1,1-dimethylethoxy)carbonyl]amino-2-methylpropane acid chloromethyl ester and stirred at 100° C. overnight. The reaction solution was condensed at a water bath set to 40° C. The residue was purified by silica gel column chromatography (15% methanol/chloroform) to obtain 83 mg (22%) of the title compound.

¹H-NMR (CDCl₃, δ):1.16(3/2H, d, J=5 Hz), 1.18(3/2H, d, J=5 Hz), 1.39 (9H, s), 2.08-2.09 (1H, m), 2.75-2.80 (2H, m), 3.02(3/2H, s), 3.03(3/2H, s), 3.17-3.37 (5H, m), 3.70(3/2H, s), 3.71(3/2H, s), 4.57-4.69(1H, m), 4.73-4.76(1H, m), 5.21(1H, br-s), 6.16(2H, br-s), 7.26-7.31 (3H, m), 7.45(1H, br-s), 7.81-7.85 (1H, m), 8.09(1H, br-s)

### Example 19

### 3-[(3-amino-2-methyl-1-oxopropoxy)methyl]-2-methyl-1-[(2,3,4,9-tetrahydro-9-methyl-4-oxo-1H-carbazol-3-yl)methyl]-1H-imidazolium Chloride Hydrochloride

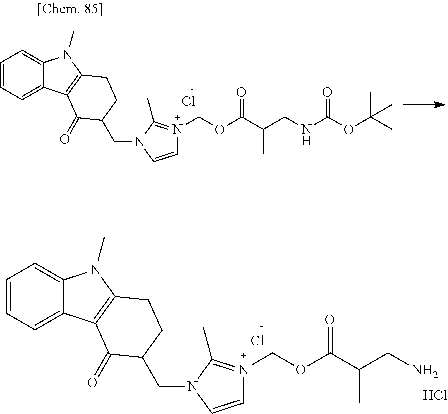

2 ml of 4 N hydrochloric acid/dioxane solution was added under cooling on ice to 2 ml of chloroform solution of 83 mg (0.15 mmol) of 3-[[3-[[(1,1-dimethylethoxy)carbonyl]amino]-2-methyl-1-oxopropoxy]methyl]-2-methy 1-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure.

Methanol and dichloromethane were added to the residue and the residue was solidified by dryness again to obtain 76 mg (quantitative) of the title compound.

¹H-NMR (DMSO-d₆, δ):1.20 (3H, d, J=7 Hz), 1.99(1H, qd, J=13, 5 Hz), 2.18-2.21 (1H, m), 2.81 (3H, s), 2.89-2.91 (1H, m), 2.98-3.21 (5H, m), 3.75 (3H, s), 4.36 (1H, dd, J=14, 7 Hz), 4.73(1H, dd, 14, 7 Hz), 6.16 (1H, d, J=12 Hz), 6.18 (1H, d, J=12 Hz), 7.21-7.29 (2H, m), 7.57 (1H, d, J=8 Hz), 7.82 (1H, d, J=2 Hz), 7.88-7.89 (1H, m), 7.98 (1H, d, J=8 Hz), 8.46(3H, br-s)

### Example 20

### [2-methyl-3-[[(ondansetron)methoxy]-3-oxopropyl]amino-chondroitin Sulfate Conjugate

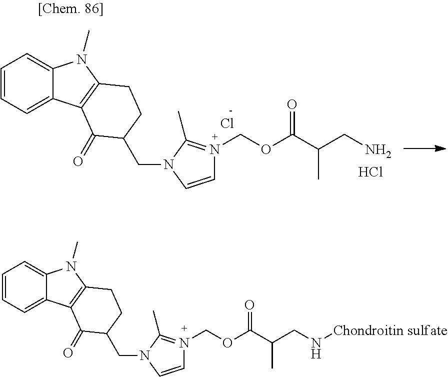

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 39 mg (0.080 mmol) of 3-[(3-amino-2-methyl-1-oxopropoxy)methyl]-2-methyl-1-[(2,3,4,9-tetrahydro-9-methyl-4-oxo-1H-carbazol-3-yl)methyl]-1H-imidazolium chloridehydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 218 mg of the title compound. Based on values of integral in ¹H-NMR, the introduction rate of ondansetron per unit of whole disaccharide (glucuronic acid) of chondroitin sulfate was 17%.

### Reference Example 51

### 3-[[(S)-2-[[(1,1-dimethylethoxy)carbonyl]amino]-3,3-dimethyl-1-oxobutoxy]methyl]-2-methyl-1-[(2,3,4,9-tetrahydro-9-methyl-4-oxo-1H-carbazol-3-yl)methyl]-1H-imidazolium Chloride

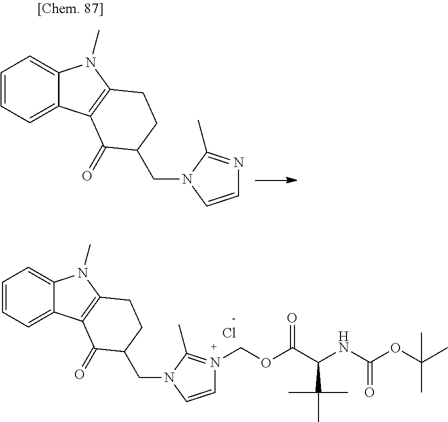

274 mg (0.94 mmol) of ondansetron was added at room temperature to an acetonitrile solution of 200 mg (0.72 mmol) of N-[(1,1-dimethylethoxy)carbonyl)]-L-tert-leucine chloromethyl ester and stirred at 100° C. overnight. The reaction solution was condensed at a water bath set to 40° C., and then the residue was purified by silica gel column chromatography (15% methanol/chloroform) to obtain 67 mg (16%) of the title compound.

¹H-NMR (CDCl₃, δ):0.95(9/2H, s), 0.96(9/2H, s), 1.37(9/2H, s), 1.38(9/2H, s), 2.00-2.08 (1H, m), 2.72-2.78(1H, m), 3.05(311, s), 3.10-3.34 (3H, m), 3.70 (3H, s), 3.99(1/2H, s), 4.00(1/2H, s), 4.50-4.54 (1H, m), 4.73-4.80(1H, m), 4.99(1H, br-s), 6.19-6.29 (2H, m), 7.26-7.33 (3H, m), 7.38(1/2H, d, J=2 Hz), 7.39(1/2H, d, J=2 Hz), 7.78(1/2H, d, J=2 Hz), 7.81(1/2H, d, J=2 Hz), 8.11-8.13(1H, m)

### Example 21

### 3-[(S)-(2-amino-3,3-dimethyl-1-oxobutoxy)methyl]-2-methyl-1-[(2,3,4,9-tetrahydro-9-methyl-4-oxo-1H-carbazol-3-yl)methyl]-1H-imidazolium Chloride Hydrochloride

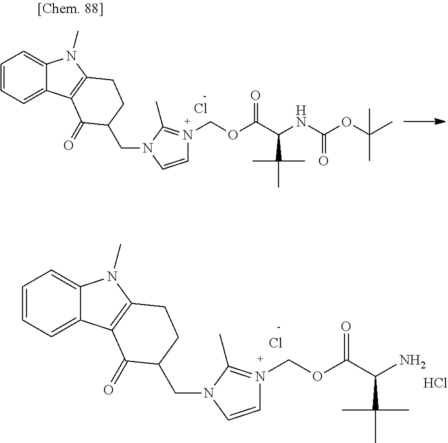

1 ml of 4 N hydrochloric acid/dioxane solution was added to 1 ml of chloroform solution of 67 mg (0.12 mmol) of 3-[[(S)-2-[[(1,1-dimethylethoxy)carbonyl]amino]-3,3-dimethyl-1-oxobutoxy]methyl]-2-methyl-1-[(2,3,4,9-tetrahydro-9-methyl-4-oxo-1H-carbazol-3-yl)methyl]-1H-imidazoliu m chloride. The reaction solution was warmed to room temperature and left to stand still for 1 hour. Thereafter, the solvent was distilled off under reduced pressure. Ethyl acetate was added to the residue and stirred. Crystals were filtered to obtain 41 mg (67%) of the title compound.

¹H-NMR (DMSO-d₆, δ):1.00 (9H, s), 1.91-2.02(1H, m), 2.18-2.20 (1H, m), 2.83(3/2H, s), 2.84(3/2H, s), 2.99-3.21 (3H, m), 3.75 (3H, s), 3.81(1H, br-s), 4.35 (1H, dd, J=15, 7 Hz), 4.75 (1H, dd, J=15, 7 Hz), 6.27-6.34 (2H, m), 7.22 (1H, t, J=8 Hz), 7.25-7.29 (1H, m), 7.57 (1H, d, J=8 Hz), 7.81 (1H, d, J=2 Hz), 7.86(1H, br-s), 7.99(1H, d, J=8 Hz), 8.72(3H, br-s)

### Example 22

### [(S)-2,2-dimethyl-1-[[(ondansetron)methoxy]carbonyl]propyl]amino-chondroitin Sulfate Conjugate

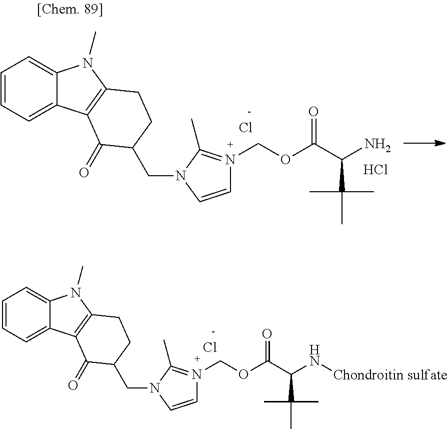

2 ml of ethanol was slowly added dropwise under stirring to 4.0 g (0.398 mmol) of 5% chondroitin sulfate aqueous solution (prepared by dissolving sodium chondroitin sulfate). To mixed liquid, a solution of 41 mg (0.080 mmol) of 3-[(S)-(2-amino-3,3-dimethyl-1-oxobutoxy)methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 212 mg of the title compound. Based on values of integral in ¹H-NMR, the introduction rate of ondansetron per unit of whole disaccharide (glucuronic acid) of chondroitin sulfate was 16%.

### Reference Example 52

### 3-[[[[1-[[(1,1-dimethylethoxy)carbonyl]amino]cyclopropyl]carbonyl]oxy]methyl]-2-methyl-1-[(2,3,4,9-tetrahydro-9-methyl-4-oxo-1H-carbazol-3-yl)methyl]-1H-imidazolium Chloride

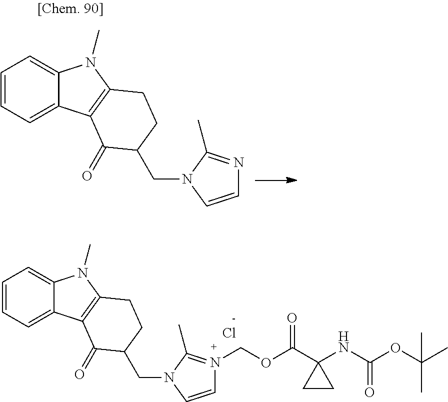

458 mg (1.56 mmol) of ondansetron was added at room temperature to an acetonitrile solution of 300 mg (0.12 mmol) of 1-[[(1,1-dimethylethoxy)carbonyl]amino]cyclopropanecarboxylic acid chloromethyl ester and stirred at 100° C. overnight. The reaction solution was condensed at a water bath set to 40° C., and then the residue was purified by silica gel column chromatography (12% methanol/chloroform) to obtain 158 mg (24%) of the title compound.

¹H-NMR (CDCl₃, δ):1.26-1.30 (4H, m), 1.42 (9H, s), 2.05(1H, qd, J=13, 5 Hz), 2.72-2.74(1H, m), 3.02 (3H, s), 3.01-3.34 (3H, m), 3.70 (3H, s), 4.48(1H, dd, J=14, 6 Hz), 4.79 (1H, dd, J=14, 6 Hz), 5.36(1H, br-s), 6.15 (1H, d, J=12 Hz), 6.21 (1H, d, J=12 Hz), 7.26-7.37 (3H, m), 7.38(1H, d, J=2 Hz), 7.67(1H, br-s), 8.13(1H, dd, J=7, 2 Hz)

### Example 23

### 3-[[[(1-aminocyclopropyl)carbonyl]oxy]methyl]-2-methyl-1-[(2,3,4,9-tetrahydro-9-methyl-4-oxo-1H-carbazol-3-yl)methyl]-1H-imidazolium Chloride Hydrochloride

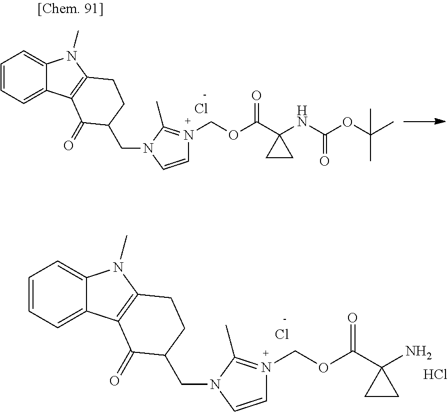

2 ml of 4 N hydrochloric acid/dioxane solution was added to 2 ml of ethyl acetate solution of 158 mg (0.29 mmol) of 3-[[[[1-[[(1,1-dimethylethoxy)carbonyl]amino]cyclopropyl]carbonyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure to obtain 118 mg (85%) of the title compound.

¹H-NMR (DMSO-d₆, δ):1.50 (4H, br-s), 1.95-2.03 (1H, m), 2.18-2.21 (1H, m), 2.79 (3H, s), 2.99-3.19 (3H, m), 3.75 (3H, s), 4.36 (1H, dd, J=15, 7 Hz), 4.73 (1H, dd, J=15, 7 Hz), 6.21(1H, d, J=12 Hz), 6.24(1H, d, J=12 Hz), 7.22(1H, t, J=8 Hz), 7.27 (1H, t, J=8 Hz), 7.57 (1H, d, J=8 Hz), 7.80 (1H, d, J=2 Hz), 7.83 (1H, d, J=2 Hz), 7.99 (1H, d, J=8 Hz), 8.99(3H, br-s)

### Example 24

### [1-[[(ondansetron)methoxy]carbonyl]cyclopropyl]amino-chondroitin Sulfate Conjugate

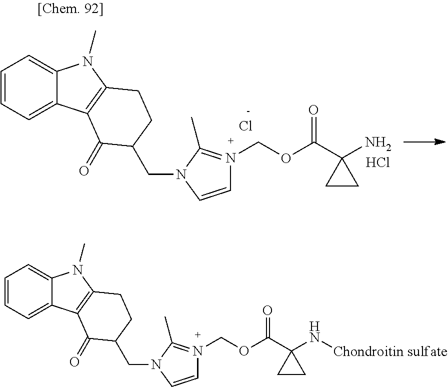

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 38 mg (0.080 mmol) of 3-[[[(1-aminocyclopropyl)carbonyl]oxy]methyl]-2-methyl-1-[(2,3,4,9-tetrahydro-9-methyl-4-oxo-1H-carbazol-3-yl)methyl]-1H-imidazolium chloride hydrochloride in 1.5 ml of ethanol was added, a solution of 38 mg (0.08 mmol) of 4-(4,6-dimethoxy-1,3,5-triazin-2-yl)-4-methylmorpholinium chloride (DMT-MM) in 0.5 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 212 mg of the title compound. Based on values of integral in ¹H-NMR, the introduction rate of ondansetron per unit of whole disaccharide (glucuronic acid) of chondroitin sulfate was 18%.

### Reference Example 53

### 3-[1-[2-[[[(1,1-dimethylethoxy)carbonyl]amino]methyl]-3-methyl-1-oxobutoxy]ethyl]-2-methyl-1-[(2,3,4,9-tetrahydro-9-methyl-4-oxo-1H-carbazol-3-yl)methyl]-1H-imidazolium Chloride

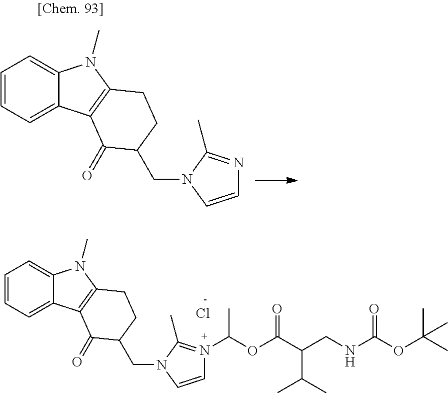

According to the method of Reference Example 32, 2-[[[(1,1-dimethylethoxy)carbonyl]amino]methyl]-3-methylbutanoic acid 1-chloroethyl ester was synthesized using 2-[[[(1,1-dimethylethoxy)carbonyl]amino]methyl]-3-methylbutanoic acid and 1-chloroethyl chlorosulfonate. 463 mg (1.58 mmol) of ondansetron was added at room temperature to an acetonitrile solution of 232 mg (0.79 mmol) of the obtained 2-[[[(1,1-dimethylethoxy)carbonyl]amino]methyl]-3-methylbutanoic acid 1-chloroethyl ester and stirred at 100° C. overnight. The reaction solution was condensed at a water bath set to 40° C. The residue was purified by silica gel column chromatography (6%→10% methanol/chloroform) to obtain 163 mg (35%) of the title compound.

¹H-NMR (CDCl₃, δ):0.82-1.02 (6H, m), 1.39-1.43 (9H, m), 1.88-1.97 (4H, m), 2.02-2.26 (1H, m), 2.50-2.57(1H, m), 2.80-2.81 (1H, m), 3.07(3/2H, s), 3.08(3/2H, s), 3.13-3.48 (5H, m), 3.70-3.73 (3H, m), 4.55-4.77 (2H, m), 4.93(3/10H, br-s), 5.10 (1/5H, br-s), 5.21(3/10H, br-s), 5.54(1/5H, br-s), 6.73-6.82 (1H, m), 7.25-7.33 (4H, m), 7.89-8.23(3H, br-s)

### Example 25

### 3-[I-[2-(aminomethyl)-3-methyl-1-oxobutoxy]ethyl]-2-methyl-1-[(2,3,4,9-tetrahydro-9-methyl-4-oxo-1H-carbazol-3-yl)methyl]-1H-imidazolium Chloride Hydrochloride

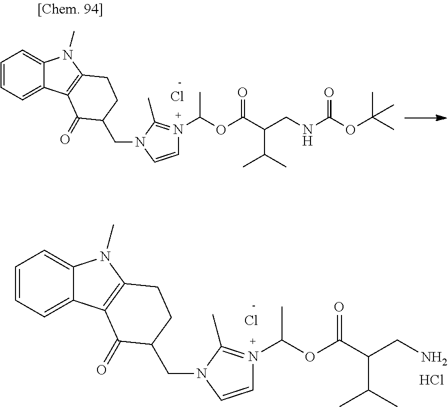

1.5 ml of 4 N hydrochloric acid/dioxane solution was added under cooling on ice to 1.5 ml of chloroform solution of 163 mg (0.28 mmol) of 3-[1-[2-[[[(1,1-dimethylethoxy)carbonyl]amino]methyl]-3-methyl-1-oxobutoxy]ethyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure to obtain 107 mg (75%) of the title compound.

¹H-NMR (DMSO-d₆, δ):0.75-0.91 (6H, m), 1.82(3/2H, d, J=6 Hz), 1.83(3/2H, d, J=6 Hz) 1.96-2.17 (3H, m), 2.70-2.71 (1H, m), 2.84(3/2H, s), 2.85(3/2H, s), 2.94-3.12 (5H, m), 3.75 (3H, s), 4.32-4.38(1H, m), 4.72-4.75 (1H, m), 6.88-6.94 (1H, m), 7.22-7.28 (2H, m), 7.56 (1H, d, J=8 Hz), 7.86-7.89 (1H, m), 7.96-8.00 (1H, m), 8.10-8.16 (1H, m), 8.30(2H, br-s)

### Example 26

### [3-methyl-2-[[1-(ondansetron)ethoxy]carbonyl]butyl]amino-chondroitin Sulfate Conjugate

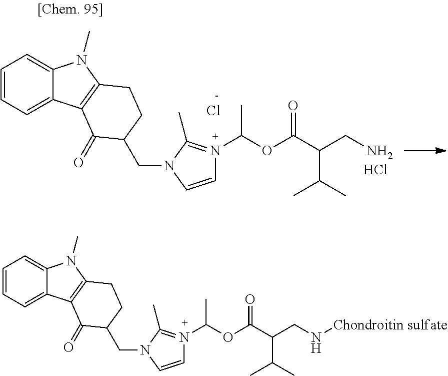

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[1-[2-(aminomethyl)-3-methyl-1-oxobutoxy]ethyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 212 mg of the title compound. Based on values of integral in ¹H-NMR, the introduction rate of ondansetron per unit of whole disaccharide (glucuronic acid) of chondroitin sulfate was 18%.

### Reference Example 54

### 3-[[2-[[(1,1-dimethylethoxy)carbonyl]amino]-2-methyl-1-oxopropoxy]methyl]-2-methy 1-1-[(2,3,4,9-tetrahydro-9-methyl-4-oxo-1H-carbazol-3-yl)methyl]-1H-imidazolium Chloride

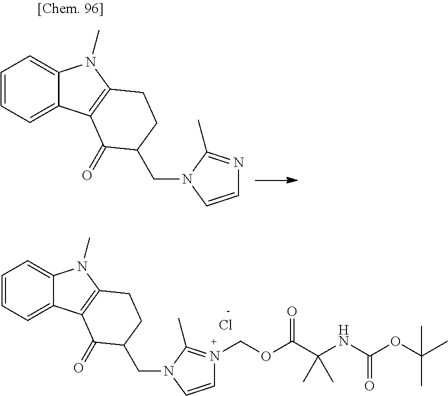

466 mg (1.59 mmol) of ondansetron was added at room temperature to an acetonitrile solution of 200 mg (0.79 mmol) of 2-[[(1,1-dimethylethoxy)carbonyl]amino]-2-methylpropane acid chloromethyl ester and stirred at 100° C. overnight. The reaction solution was condensed at a water bath set to 40° C., and then the residue was purified by silica gel column chromatography (15% methanol/chloroform) to obtain 248 mg (58%) of the title compound.

¹H-NMR (CDCl₃, δ):1.32 (9H, s), 1.45 (3H, s), 1.46 (3H, s), 2.03(1H, qd, J=13, 5 Hz), 2.70-2.75 (1H, m), 3.03 (3H, s), 3.09-3.33 (3H, m), 3.69 (3H, s), 4.48 (1H, dd, J=14, 6 Hz), 4.75 (1H, dd, J=14, 6 Hz), 4.90(1H, br-s), 6.21 (1H, d, J=12 Hz), 6.27(1H, d, J=12 Hz), 7.26-7.33 (3H, m), 7.43(1H, d, J=2 Hz), 7.73(1H, d, J=2 Hz), 8.11-8.13 (1H, m)

### Example 27

### 3-[(2-amino-2-methyl-1-oxopropoxy)methyl]-2-methyl-1-[(2,3,4,9-tetrahydro-9-methyl-4-oxo-1H-carbazol-3-yl)methyl]-1H-imidazolium Chloride Hydrochloride

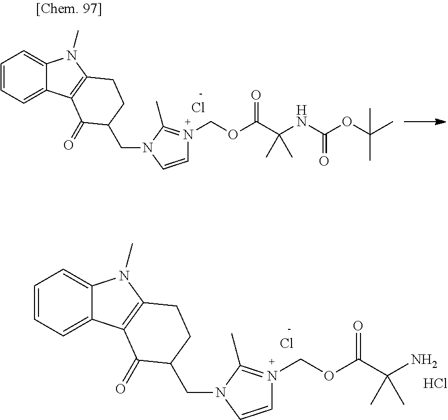

1.5 ml of 4 N hydrochloric acid/dioxane solution was added to 1.5 ml of ethyl acetate solution of 248 mg (0.45 mmol) of 3-[[2-[[(1,1-dimethylethoxy)carbonyl]amino]-2-methyl-1-oxopropoxy]methyl]-2-methy 1-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 284 mg (quantitative) of the title compound.

¹H-NMR (DMSO-d₆, δ):1.52 (6H, s), 1.95-2.03 (1H, m), 2.18-2.22 (1H, m), 2.82 (3H, s), 2.99-3.21 (3H, m), 3.75 (3H, s), 4.34(1H, dd, J=15, 7 Hz), 4.73 (1H, dd, J=15, 7 Hz), 6.26 (1H, d, J=11 Hz), 6.29 (1H, d, J=11 Hz), 7.20-7.28 (2H, m), 7.56 (1H, d, J=8 Hz), 7.80 (1H, d, J=2 Hz), 7.86 (1H, d, J=2 Hz), 7.96 (1H, d, J=8 Hz), 8.95(3H, br-s)

### Example 28

### [1,1-dimethyl-2-[(ondansetron)methoxy]-2-oxoethyl]amino-chondroitin Sulfate Conjugate

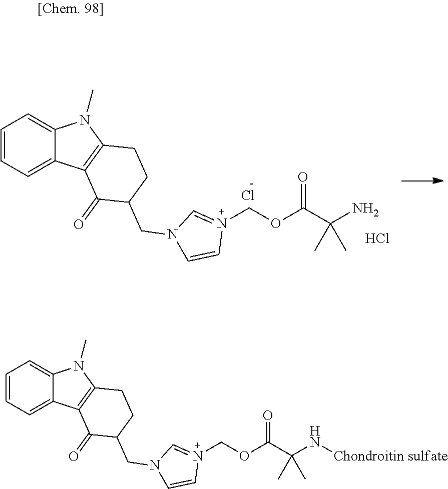

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 39 mg (0.080 mmol) of 3-[(2-amino-2-methyl-1-oxopropoxy)methyl]-2-methyl-1-[(2,3,4,9-tetrahydro-9-methyl-4-oxo-1H-carbazol-3-yl)methyl]-1l-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 152 mg of the title compound. Based on values of integral in ¹H-NMR, the introduction rate of ondansetron per unit of whole disaccharide (glucuronic acid) of chondroitin sulfate was 0.3%.

### Reference Example 55

### 3-[[2-[[[(1,1-dimethylethoxy)carbonyl]amino]methyl]-1-oxobutoxy]methyl]-2-methyl-1-[(2,3,4,9-tetrahydro-9-methyl-4-oxo-1H-carbazol-3-yl)methyl]-1H-imidazolium Chloride

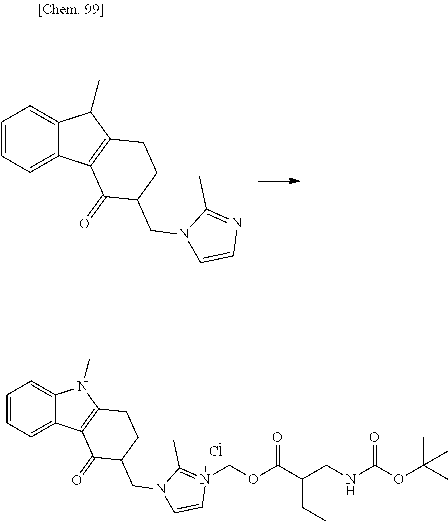

543 mg (1.85 mmol) of ondansetron was added at room temperature to an acetonitrile solution of 287 mg (0.93 mmol) of 2-[[[(1,1-dimethylethoxy)carbonyl]amino]methyl]-2-methylbutanoic acid chloromethyl ester and stirred at 100° C. overnight. The reaction solution was condensed at a water bath set to 40° C. The residue was purified by silica gel column chromatography (12% methanol/chloroform) to obtain 517 mg (99%) of the title compound.

¹H-NMR (CDCl₃, δ):0.89-0.90 (3H, m), 1.37(9/2H, s), 1.39(9/2H, s), 1.54-1.55 (211, m), 2.08(1H, br-s), 2.64-2.76 (2H, m), 3.03-3.39 (8H, m), 3.71 (3H, s), 4.56-4.60 (1H, m), 4.74(1H, dd, J=14, 7 Hz), 5.13 (1H, br-s), 6.14 (1H, d, J=14 Hz), 6.17 (1H, d, J=14 Hz), 7.26-7.33 (3H, m), 7.41-7.42 (1H, m), 7.83-7.85 (1H, m), 8.08(1H, br-s)

### Example 29

### 3-[[2-(aminomethyl)-1-oxobutoxy]methyl]-2-methyl-1-[(2,3,4,9-tetrahydro-9-methyl-4-oxo-1H-carbazol-3-yl)methyl]-1H-imidazolium Chloride Hydrochloride

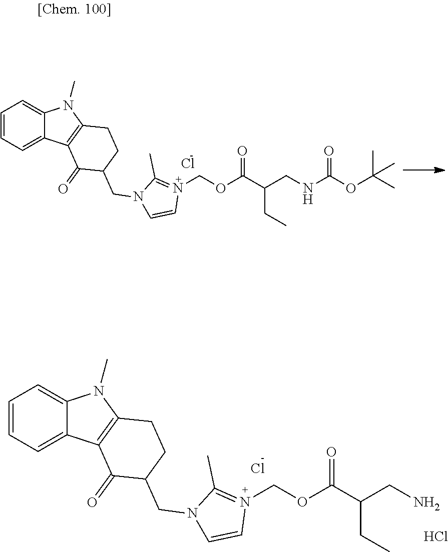

3 ml of 4 N hydrochloric acid/dioxane solution was added to 3 ml of chloroform solution of 517 mg (0.92 mmol) of 3-[[2-[[[(1,1-dimethylethoxy)carbonyl]amino]methyl]-1-oxobutoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 439 mg (95%) of the title compound.

¹H-NMR (DMSO-d₆, δ):0.80(3/2H, t, J=8 Hz), 0.81(3/2H, t, J=8 Hz), 1.57-1.68 (2H, m), 1.91-2.02 (1H, m), 2.14-2.20 (1H, m), 2.80-2.84 (4H, m), 2.92-3.20 (5H, m), 3.74(3/2H, s), 3.75(3/2H, s), 4.35(1/2H, dd, J=14, 7 Hz), 4.36(1/2H, dd, J=14, 7 Hz), 4.73(1H, dd, 14, 7 Hz), 6.14-6.21 (2H, m), 7.20-7.23 (1H, m), 7.27(1H, td, J=8, 2 Hz), 7.54-7.57 (1H, m), 7.80 (1H, d, J=2 Hz), 7.87-7.88(1H, m), 7.97-8.00 (1H, m), 8.30(3H, br-s)

### Example 30

### [2-[[(ondansetron)methoxy]carbonyl]butyl]amino-chondroitin Sulfate Conjugate

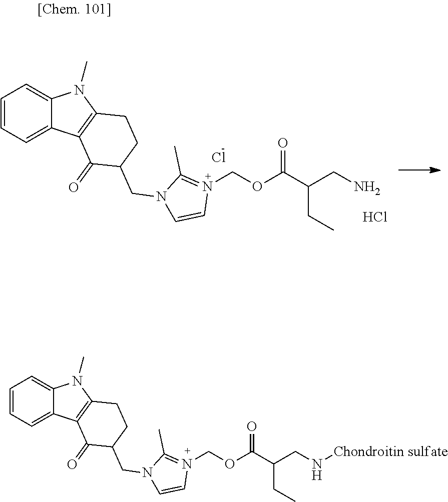

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 40 mg (0.080 mmol) of 3-[[2-(aminomethyl)-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 2 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 50 mg of the title compound. Based on values of integral in ¹H-NMR, the introduction rate of ondansetron per unit of whole disaccharide (glucuronic acid) of chondroitin sulfate was 14%.

### Reference Example 56

### 1-[2-[(4-chlorophenyl)phenyl methoxy]ethyl]-1-[[2-[[[(1,1-dimethylethoxy)carbonyl]amino]methyl]-3-methyl-1-oxobutoxy]methyl]piperidinium Chloride

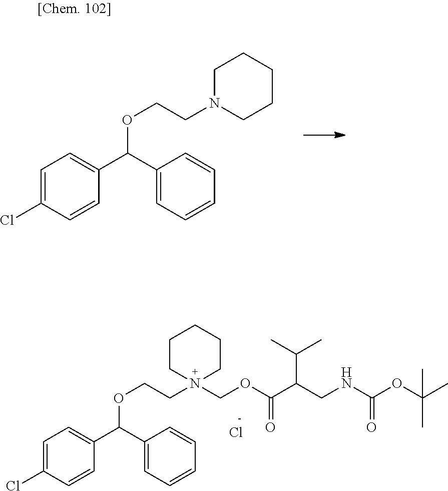

472 mg (1.43 mmol) of cloperastine was added at room temperature to a dichloromethane solution of 200 mg (0.71 mmol) of 2-[[[(1,1-dimethylethoxy)carbonyl]amino]methyl]-3-methylbutanoic acid chloromethyl ester, and the reaction solution was condensed at 70° C. over 1 hour and then stirred at the same temperature overnight. The residue was purified by silica gel column chromatography (2→10% methanol/chloroform) to obtain 367 mg (85%) of the title compound.

¹H-NMR (CDCl₃, δ):0.94 (3H, d, J=7 Hz), 0.97 (3H, d, J=7 Hz), 1.40 (9H, s), 1.77-1.99 (7H, m), 2.59(1H, br-s), 3.29-3.43 (2H, m), 3.65-3.76 (2H, m), 3.95-4.05 (4H, m), 4.16-4.24 (2H, m), 5.07(1H, br-s), 5.48(1H, s), 5.71-5.84 (2H, m), 7.25-7.35 (9H, m)

### Example 31

### 1-[2-[(4-chlorophenyl)phenyl methoxy]ethyl]-1-[[2-(aminomethyl)-3-methyl-1-oxobutoxy]methyl]piperidinium Chloridehydrochloride

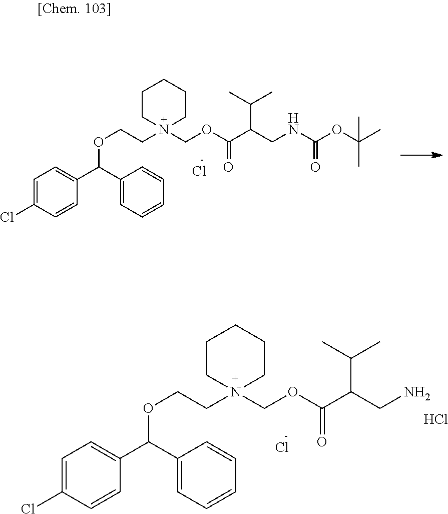

2 ml of 4 N hydrochloric acid/dioxane solution was added under cooling on ice to 2 ml of ethyl acetate solution of 367 mg (0.60 mmol) of 1-[2-[(4-chlorophenyl)phenyl methoxy]ethyl]-1-[[2-[[[(1,1-dimethylethoxy)carbonyl]amino]methyl]-3-methyl-1-oxobutoxy]methyl]piperidinium chloride. The reaction solution was warmed to room temperature and left to stand still for 2 hours. Thereafter, the solvent was distilled off under reduced pressure. Ethyl acetate was added to the residue and stirred for 1 hour. Crystals were filtered to obtain 229 mg (70%) of the title compound.

¹H-NMR (DMSO-d₆, δ):0.92-0.99 (6H, m), 1.64-1.93 (6H, m), 2.06-2.14 (1H, m), 2.99-3.01 (3H, m), 3.60-3.63 (4H, m), 3.89(4H, br-s), 5.46-5.70 (3H, m), 7.30-7.43 (9H, m), 8.57(3H, br-s)

### Example 32

### [2-[[(cloperastine)methoxy]carbonyl]-3-methylbutyl]amino-chondroitin Sulfate Conjugate

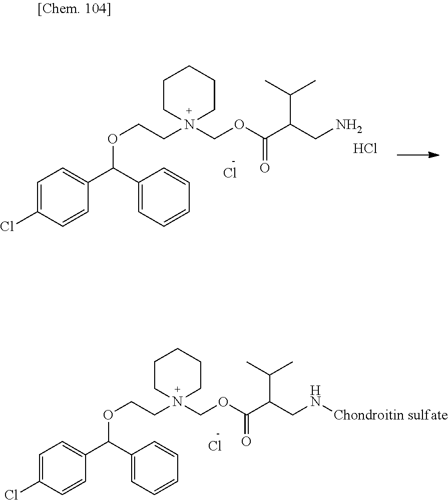

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 44 mg (0.080 mmol) of 1-[2-[(4-chlorophenyl)phenyl methoxy]ethyl]-1-[[2-(aminomethyl)-3-methyl-1-oxobutoxy]methyl]piperidinium chloridehydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (6 ml) until immediately before the reaction solution became cloudy. The reaction solution was added dropwise under stirring to 8 ml of 90% ethanol, and 6 ml of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 214 mg of the title compound. Based on values of integral in ¹H-NMR, the introduction rate of cloperastine per unit of whole disaccharide (glucuronic acid) of chondroitin sulfate was 16%.

### Reference Example 57

### N-[[2-[[(1,1-dimethylethoxy)carbonyl]amino]methyl]-3-methyl-1-oxobutoxy]methyl]-N,N,α-trimethyl-10H-phenothiazin-10-ethanaminium Chloride

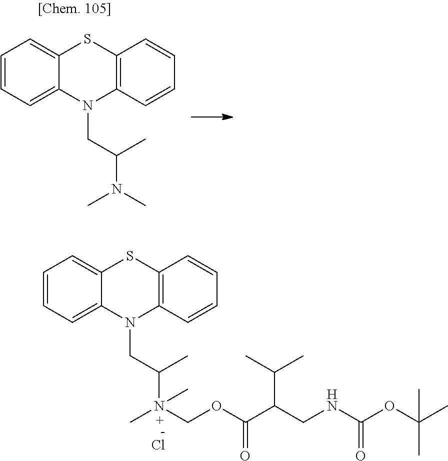

400 mg (1.40 mmol) of promethazine was added at room temperature to a dichloromethane solution of 197 mg (0.70 mmol) of 2-[[[(1,1-dimethylethoxy)carbonyl]amino]methyl]-3-methylbutanoic acid chloromethyl ester, and the reaction solution was condensed at 70° C. over 1 hour and then stirred at the same temperature overnight. The residue was purified by silica gel column chromatography (5→20% methanol/chloroform) to obtain 369 mg (94%) of the title compound.

¹H-NMR (CDCl₃, δ):0.84(3/2H, d, J=7 Hz), 0.86(3/2H, d, J=7 Hz), 0.88(3/2H, d, J=7 Hz), 0.91(3/2H, d, J=7 Hz), 1.37(9/2H, s), 1.40(9/2H, s), 1.66(3/2H, d, J=7 Hz), 1.67(3/2H, d, J=7 Hz), 1.74-1.86(l H, m), 2.43-2.44 (1H, m), 3.18-3.23 (1H, m), 3.32-3.38 (1H, m), 3.42(3/2H, s), 3.45(3/2H, s), 3.52(3/2H, s), 3.57(3/2H, s), 4.13-4.15 (1H, m), 4.28-4.34 (1H, m), 4.90(1/2H, d, J=6 Hz), 4.93(1/2H, d, J=6 Hz), 5.14(1/2H, br-s), 5.22(1/2H, br-s), 5.73-5.77 (1H, m), 5.86(1/2H, d, J=9 Hz), 5.91(1/2H, d, J=9 Hz), 7.01-7.05 (2H, m), 7.13-7.16 (2H, m), 7.22-7.29 (4H, m)

### Example 33

### N-[[2-(aminomethyl)-3-methyl-1-oxobutoxy]methyl]-N,N,α-trimethyl-1 OH-phenothiazin-10-ethanaminium Chloride Hydrochloride

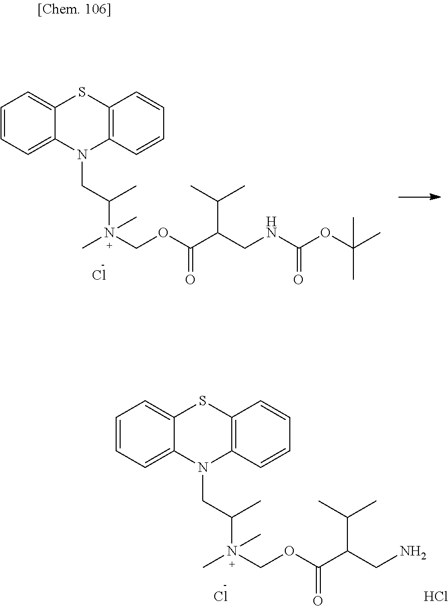

4 ml of 4 N hydrochloric acid/dioxane solution was added to 4 ml of ethyl acetate solution of 369 mg (0.66 mmol) of N-[[2-[[[(1,1 dimethylethoxy)carbonyl]amino]methyl]-3-methyl-1-oxobutoxy]methyl]-N,N,α-trimethyl-10H-phenothiazin-10-ethanaminium chloride. The reaction solution was warmed to room temperature and left to stand still for 2 hours. Thereafter, the solvent was distilled off under reduced pressure. Ethyl acetate was added to the residue and stirred for 1 hour. Crystals were filtered to obtain 298 mg (91%) of the title compound.

¹H-NMR (DMSO-d₆, δ):0.82 (3H, d, J=7 Hz), 0.86-0.89 (3H, m), 1.45 (3H, d, J=7 Hz), 1.91-2.00(1H, m), 2.79(1/2H, q, J=5 Hz), 2.80(1/2H, q, J=5 Hz), 2.95-3.10 (2H, m), 3.10-3.30 (6H, m), 3.89-3.97 (1H, m), 4.15-4.21(1H, m), 4.69-4.73 (1H, m), 5.42(1/2H, d, J=9 Hz), 5.45(1/2H, d, J=9 Hz), 5.55(1/2H, d, J=9 Hz), 5.56(1/2H, d, J=9 Hz), 7.06-7.09 (2H, m), 7.26-7.35 (6H, m), 8.27(3H, br-s)

### Example 34

### [3-methyl-2-[[(promethazine)methoxy]carbonyl]butyl]amino-chondroitin Sulfate Conjugate

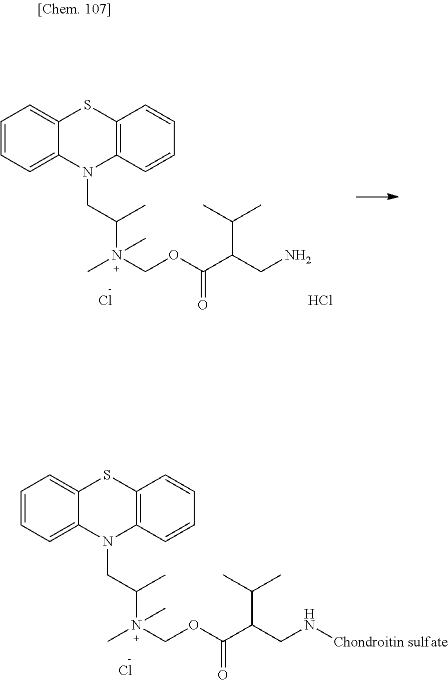

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 44 mg (0.080 mmol) of N-[[2-(aminomethyl)-3-methyl-1-oxobutoxy]methyl]-N,N,α-trimethyl-1 0H-phenothiazin-10-ethanaminium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (3 ml) until immediately before the reaction solution became cloudy. The reaction solution was added dropwise under stirring to 8 ml of 90% ethanol, and 7 ml of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 177 mg of the title compound. Based on values of integral in ¹H-NMR, the introduction rate of promethazine per unit of whole disaccharide (glucuronic acid) of chondroitin sulfate was 18%.

### Reference Example 58

### N-[[3-[[(1,1-dimethylethoxy)carbonyl]amino]-2,2-dimethyl-1-oxopropoxy]methyl]-N,N,α-trimethyl-10H-phenothiazin-10-ethanaminium Chloride

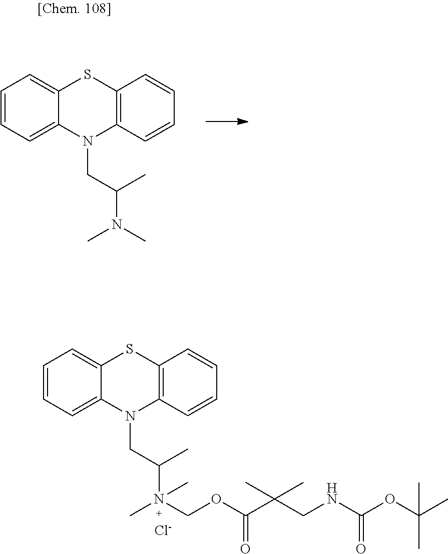

400 mg (1.40 mmol) of promethazine was added at room temperature to a dichloromethane solution of 186 mg (0.70 mmol) of 3-[[(1,1-dimethylethoxy)carbonyl]amino]-2,2-dimethylpropane acid chloromethyl ester, and the reaction solution was condensed at 70° C. over 1 hour and then stirred at the same temperature overnight. The residue was purified by silica gel column chromatography (5→10% methanol/chloroform) to obtain 185 mg (48%) of the title compound.

¹H-NMR (CDCl₃, δ):1.01 (3H, s), 1.06 (3H, s), 1.40 (9H, s), 1.68 (3H, d, J=7 Hz), 3.04-3.14 (2H, m), 3.46 (3H, s), 3.51 (3H, s), 4.12(1H, br-s), 4.24(1H, dd, J=16, 5 Hz), 4.90(1H, dd, J=16, 7 Hz), 5.05(1H, br-s), 5.64 (1H, d, J=8 Hz), 5.76 (1H, d, J=8 Hz), 7.01-7.04 (2H, m), 7.10-7.11 (2H, m), 7.22-7.28 (4H, m)

### Example 35

### N-[(3-amino-2,2-dimethyl-1-oxopropoxy)methyl]-N,N,α-trimethyl-10H-phenothiazin-10-ethanaminium C

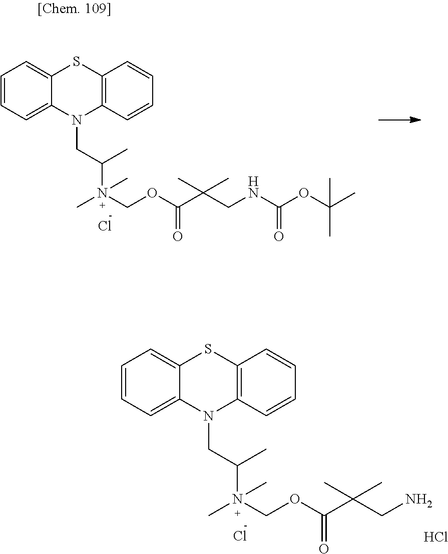

1 ml of 4 N hydrochloric acid/dioxane solution was added under cooling on ice to 1 ml of chloroform solution of 176 mg (0.32 mmol) of N-[[3-[[(1,1-dimethylethoxy)carbonyl]amino]-2,2-dimethyl-1-oxopropoxy]methyl]-N,N,α-trimethyl-10H-phenothiazin-10-ethanaminium chloride. The reaction solution was warmed to room temperature and left to stand still for 1.5 hours. Thereafter, the solvent was distilled off under reduced pressure. Diethyl ether was added to the residue and stirred for 1 hour. Crystals were filtered to obtain 120 mg (77%) of the title compound.

¹H-NMR (DMSO-d₆, δ):1.18 (3H, s), 1.21 (3H, s), 1.45 (3H, d, J=7 Hz), 2.99(2H, br-s), 3.19 (3H, s), 3.24 (3H, s), 3.91-3.94 (1H, m), 4.18(1H, dd, J=15, 8 Hz), 4.70-4.72 (1H, m), 5.42 (1H, d, J=8 Hz), 5.48(1H, d, J=8H2), 7.05-7.08 (2H, m), 7.26-7.32 (6H, m), 8.43(3H, br-s)

### Example 36

### [2-methyl-2-[[(promethazine)methoxy]carbonyl]propyl]amino-chondroitin Sulfate Conjugate

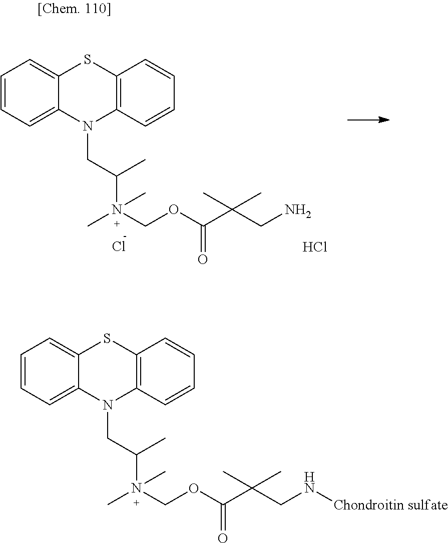

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 44 mg (0.080 mmol) of N-[(3-amino-2,2-dimethyl-1-oxopropoxy)methyl]-N,N,α-trimethyl-10H-phenothiazin-10-ethanaminium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 201 mg of the title compound. Based on values of integral in 1H-NMR, the introduction rate of promethazine per unit of whole disaccharide (glucuronic acid) of chondroitin sulfate was 20%.

### Reference Example 59

### 3-[[3-phenyl-2-[[[(1,1-dimethylethoxy)carbonyl]amino]methyl]-1-oxobutoxy]methyl]-2-methyl-1-[(2,3,4,9-tetrahydro-9-methyl-4-oxo-1H-carbazol-3-yl)methyl]-1H-imidazolium Chloride

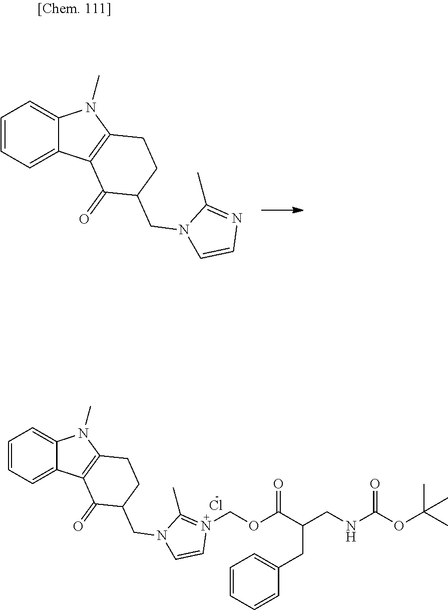

575 mg (1.96 mmol) of ondansetron was added at room temperature to an acetonitrile solution of 320 mg (0.98 mmol) of 2-benzyl-3-[[[(1,1-dimethylethoxy)carbonyl]amino]methyl]propanoic acid chloromethyl ester and stirred at 100° C. overnight. The reaction solution was condensed at a water bath set to 40° C. The residue was purified by silica gel column chromatography (13% methanol/chloroform) to obtain 371 mg (61%) of the title compound.

¹H-NMR (CDCl₃, δ):1.39(9/2H, s), 1.41(9/2H, s), 1.98-2.07 (1H, m), 2.74-2.86 (7H, m), 2.95-3.45 (5H, m), 3.69(3/2H, s), 3.70(3/2H, s), 4.41-4.52(1H, m), 4.65-4.74 (1H, m), 5.12(1H, br-s), 5.98-6.07 (2H, m), 7.05-7.09 (2H, m), 7.17-7.33 (7H, m), 7.72-7.74 (1H, m), 8.09 (1H, d, J=7 Hz)

### Example 37

### 3-[[2-(aminomethyl)-3-phenyl-1-oxopropoxy]methyl]-2-methyl-1-[(2,3,4,9-tetrahydro-9-methyl-4-oxo-1H-carbazol-3-yl)methyl]-1H-imidazolium Chloride Hydrochloride

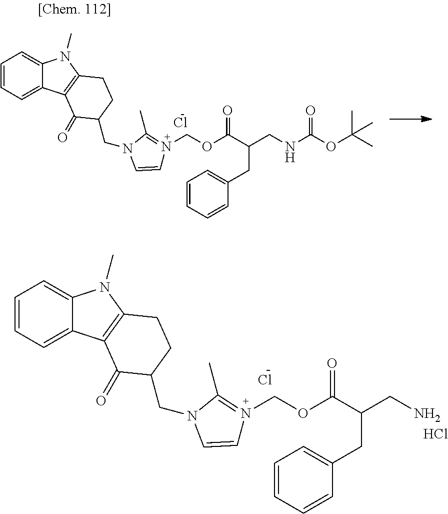

2 ml of 4 N hydrochloric acid/dioxane solution was added under cooling on ice to 2 ml of dichloromethane solution of 371 mg (0.60 mmol) of 3-[[3-phenyl-2-[[[(1,1-dimethylethoxy)carbonyl]amino]methyl]-1-oxobutoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1.5 hours. Thereafter, the solvent was distilled off under reduced pressure. Ethyl acetate was added to the residue and stirred for 1 hour. Crystals were filtered to obtain 290 mg (87%) of the title compound.

¹H-NMR (DMSO-d₆, δ):1.91-2.01 (1H, m), 2.14-2.21 (1H, m), 2.64-2.66 (3H, m), 2.82-3.27 (8H, m), 3.74(3/2H, s), 3.75(3/2H, s), 4.34-4.39(1H, m), 4.63-4.75 (1H, m), 6.05-6.15 (2H, m), 7.11-7.13 (2H, m), 7.19-7.28 (4H, m), 7.55-7.57 (2H, m), 7.75-7.79 (2H, m), 7.97-8.00(1H, m), 8.53(3H, br-s)

### Example 38

### [3-phenyl-2-[[(ondansetron)methoxy]carbonyl]propyl]amino-chondroitin Sulfate Conjugate

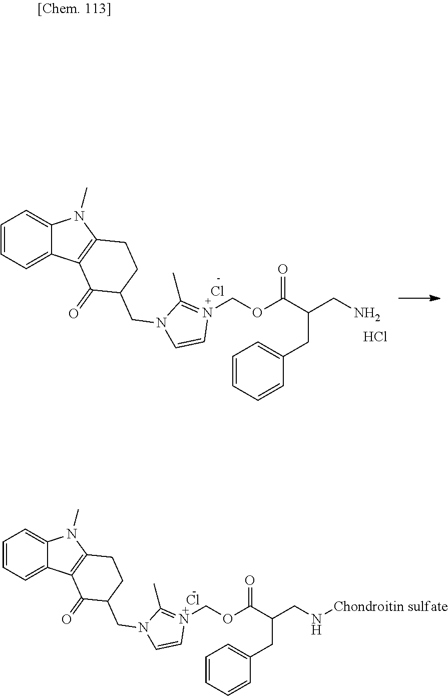

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 45 mg (0.080 mmol) of 3-[[2-(aminomethyl)-3-phenyl-1-oxopropoxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 d of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 50 mg of the title compound. Based on values of integral in ¹H-NMR, the introduction rate of ondansetron per unit of whole disaccharide (glucuronic acid) of chondroitin sulfate was 25%.

### Reference Example 60

### 3-[[[7-[[(1,1-dimethylethoxy)carbonyl]amino]-1-oxoheptyl]oxy]methyl]-2-methyl-1-[(2,3,4,9-tetrahydro-9-methyl-4-oxo-1H-carbazol-3-yl)methyl]-1H-imidazolium Chloride

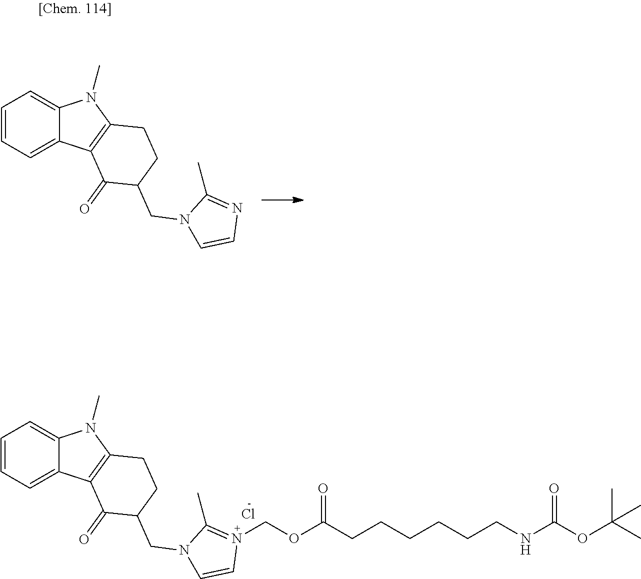

379 mg (1.29 mmol) of ondansetron was added at room temperature to an acetonitrile solution of 253 mg (0.86 mmol) of 7-[[(1,1-dimethylethoxy)carbonyl]amino]heptanoic acid chloromethyl ester and stirred at 100° C. overnight. The reaction solution was condensed at a water bath set to 40° C. The residue was purified by silica gel column chromatography (13-420% methanol/chloroform) to obtain 387 mg (82%) of the title compound.

¹H-NMR (CDCl₃, δ):1.29-1.31 (5H, m), 1.43 (9H, s), 1.58-1.64 (3H, m), 2.25-2.30(1H, m), 2.39 (2H, t, J=8 Hz), 2.74-2.77(1H, m), 2.95-3.33 (8H, m), 3.69 (3H, s), 4.56-4.62 (2H, m), 4.75 (1H, dd, J=14, 7 Hz), 6.12 (1H, d, J=12 Hz), 6.14 (1H, d, J=12 Hz), 7.19-7.34 (3H, m), 7.42 (1H, d, J=2 Hz), 7.88(1H, d, J=2 Hz), 8.08-8.10(1H, m)

### Example 39

### 3-[[(7-amino-1-oxoheptyl)oxy]methyl]-2-methyl-1-[(2,3,4,9-tetrahydro-9-methyl-4-oxo-1H-carbazol-3-yl)methyl]-1H-imidazolium Chloride Hydrochloride

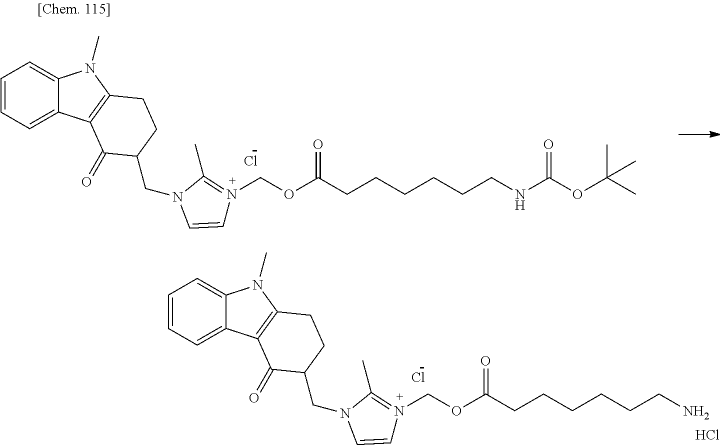

3 ml of 4 N hydrochloric acid/dioxane solution was added under cooling on ice to 3 ml of chloroform solution of 387 mg (0.70 mmol) of 3-[[[7-[[(1,1-dimethylethoxy)carbonyl]amino]-1-oxoheptyl]oxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 0.5 hour. Thereafter, the solvent was distilled off under reduced pressure. Ethyl acetate was added to the residue and stirred for 1 hour. Crystals were filtered to obtain 269 mg (74%) of the title compound.

¹H-NMR (DMSO-d₆, δ):1.25-1.31 (4H, m), 1.52-1.57 (4H, m), 1.91-2.03 (1H, m), 2.14-2.22(1H, m), 2.42 (2H, t, J=7 Hz), 2.69-2.75 (2H, m), 2.77 (3H, s), 2.96-3.21 (3H, m), 3.75 (3H, s), 4.37 (1H, dd, J=14, 7 Hz), 4.72 (1H, dd, J=14, 7 Hz), 6.14 (2H, s), 7.22(1H, t, J=8 Hz), 7.27 (1H, t, J=8 Hz), 7.55-7.58 (1H, m), 7.84 (2H, s), 7.97-8.00 (1H, m), 8.16(3H, br-s)

### Example 40

### [7-[(ondansetron)methoxy]-1-oxyheptyl]amino-chondroitin Sulfate Conjugate

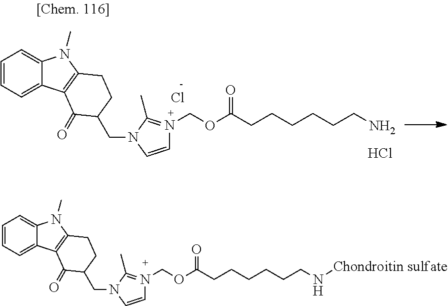

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 42 mg (0.080 mmol) of 3-[[(7-amino-1-oxoheptyl)oxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 50 mg of the title compound. Based on values of integral in ¹H-NMR, the introduction rate of ondansetron per unit of whole disaccharide (glucuronic acid) of chondroitin sulfate was 11%.

### Reference Example 61

### 3-[[2-[[(1,1-dimethylethoxy)carbonyl]amino]-2-ethyl-1-oxobutoxy]methyl]-2-methyl-1-[(2,3,4,9-tetrahydro-9-methyl-4-oxo-1H-carbazol-3-yl)methyl]-1H-imidazolium Chloride

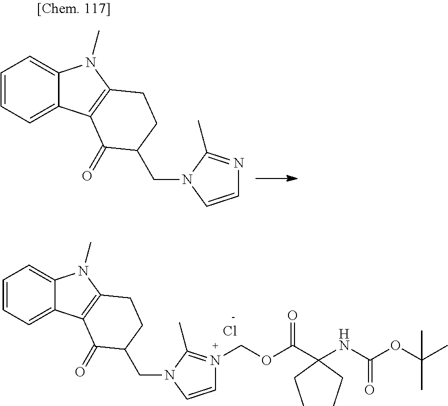

437 mg (1.49 mmol) of ondansetron was added at room temperature to an acetonitrile solution of 278 mg (0.99 mmol) of 2-[[(1,1-dimethylethoxy)carbonyl]amino-2-ethylbutanoic acid chloromethyl ester and stirred at 100° C. overnight. The reaction solution was condensed at a water bath set to 40° C. The residue was purified by silica gel column chromatography (15% methanol/chloroform) to obtain 308 mg (54%) of the title compound.

¹H-NMR (CDCl₃, δ):0.75 (6H, t, J=7 Hz), 1.35 (9H, s), 1.80-2.06 (5H, m), 2.69-2.74 (1H, m), 3.05 (3H, s), 3.09-3.33 (3H, m), 3.69 (3H, s), 4.49 (1H, dd, J=14, 6 Hz), 4.74 (1H, dd, J=14, 6 Hz), 4.91(1H, br-s), 6.23 (1H, d, J=12 Hz), 6.29(1H, d, J=12 Hz), 7.26-7.32 (3H, m), 7.42 (1H, d, J=2 Hz), 7.78 (1H, d, J=2 Hz), 8.10-8.12(1H, m)

### Example 41

### 3-[(2-amino-2-ethyl-1-oxobutoxy)methyl]-2-methyl-1-[(2,3,4,9-tetrahydro-9-methyl-4-oxo-1H-carbazol-3-yl)methyl]-1H-imidazolium Chloride Hydrochloride

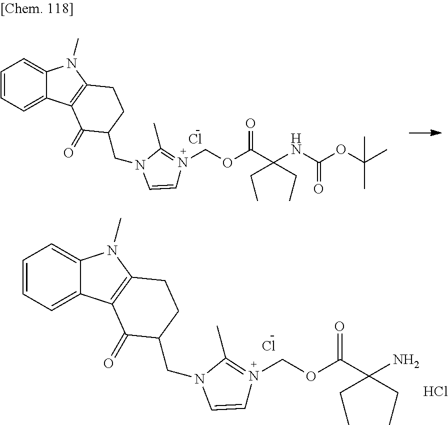

1.5 ml of 4 N hydrochloric acid/dioxane solution was added under cooling on ice to 1.5 ml of chloroform solution of 293 mg (0.51 mmol) of 3-[[2-[[(1,1-dimethylethoxy)carbonyl]amino]-2-ethyl-1-oxobutoxy]methyl]-2-methyl-1-[(2,3,4,9-tetrahydro-9-methyl-4-oxo-1H-carbazol-3-yl)methyl]-1H-imidazolium chloride. The reaction solution was warmed to room temperature and left to stand still for 1 hour. Thereafter, the solvent was distilled off under reduced pressure. Ethyl acetate was added to the residue and stirred for 1 hour. Crystals were filtered to obtain 213 mg (77%) of the title compound.

¹H-NMR (DMSO-d₆, δ):0.86 (6H, t, J=8 Hz), 1.87(4H, q, J=8 Hz), 1.94-2.02(1H, m), 2.16-2.19 (1H, m), 2.83 (3H, s), 2.99-3.05 (1H, m), 3.14-3.19 (2H, m), 3.75 (3H, s), 4.37(1H, dd, J=14, 7 Hz), 4.74(1H, dd, J=14, 7 Hz), 6.32(1H, d, 12 Hz), 6.35(1H, d, J=12 Hz), 7.20-7.28 (2H, m), 7.56 (1H, d, J=8 Hz), 7.82 (1H, d, J=2 Hz), 7.88 (1H, d, J=2 Hz), 7.97 (1H, d, J=8 Hz), 8.79(3H, br-s)

### Example 42

### [1-ethyl-1-[[(ondansetron)methoxy]carbonyl]propyl]amino-chondroitin Sulfate Conjugate

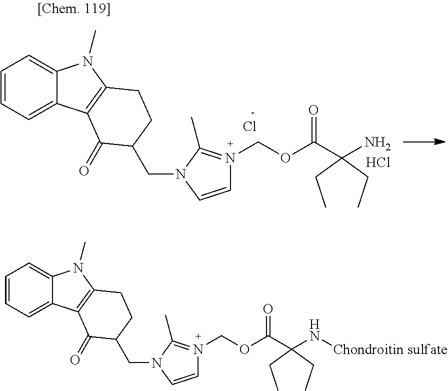

2 ml of ethanol was slowly added dropwise under stirring to 4.0 g (0.398 mmol) of 5% chondroitin sulfate aqueous solution (prepared by sodium chondroitin sulfate). To the mixed liquid, a solution of 41 mg (0.080 mmol) of 3-[(2-amino-2-ethyl-1-oxobutoxy)methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 ml) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 50 mg of the title compound. Based on values of integral in ¹H-NMR, the introduction rate of ondansetron per unit of whole disaccharide (glucuronic acid) of chondroitin sulfate was 11%.

### Example 43

### [3,3-dimethyl-2-[[(ondansetron)methoxy]carbonyl]butyl]amino-hyaluronic Acid Conjugate

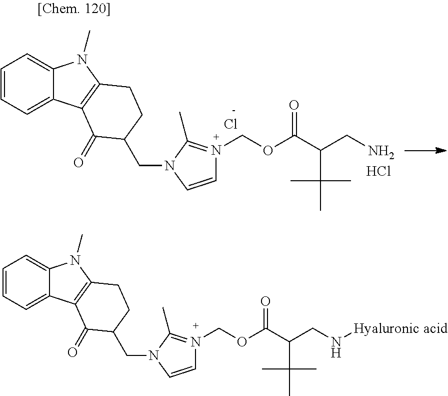

10 ml of ethanol was slowly added dropwise under stirring to 10 g (0.249 mmol) of 1% hyaluronic acid aqueous solution (prepared by dissolving sodium hyaluronate. To the mixed liquid, a solution of 3.3 mg (0.006 mmol) of 3-[[2-(aminomethyl)-3,3-dimethyl-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 2.9 mg (0.006 mmol) of 4-(4,6-dimethoxy-1,3,5-triazin-2-yl)-4-methylmorpholinium chloride (DMT-MM) in 1 ml of ethanol was then added, 0.5 ml of ethanol and 2.5 ml of water were further added, and the resultant liquid was stirred at room temperature overnight. 1.5 ml of 20% sodium chloride aqueous solution and 30 ml of ethanol were added to the reaction solution to form precipitates and the supernatant of the suspension was removed. 12 ml of ethanol was further added thereto and the supernatant was removed. Thereafter, washing with 90% ethanol was performed two times, washing with ethanol was performed two times, and washing with diethyl ether was further performed two times. The obtained precipitates were dried overnight using a vacuum pump to obtain 89 mg of the title compound. Based on values of integral in ¹H-NMR, the introduction rate of ondansetron per unit of whole disaccharide (glucuronic acid) of hyaluronic acid was 3%.

### Example 44

### [3,3-dimethyl-2-[[(ondansetron)methoxy]carbonyl]butyl]amino-carboxymethyl Cellulose Conjugate

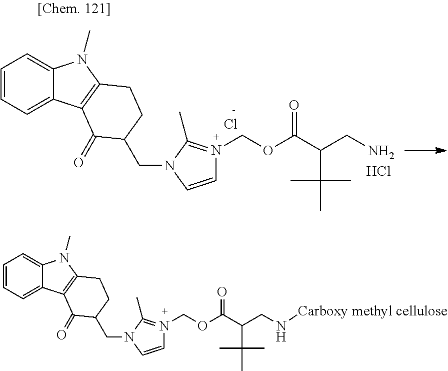

10 ml of ethanol was slowly added dropwise under stirring to 10 g (0.426 mmol) of 1% carboxymethyl cellulose aqueous solution (prepared by dissolving sodium carboxymethyl cellulose). To the mixed liquid, a solution of 5.6 mg (0.0106 mmol) of 3-[[2-(aminomethyl)-3,3-dimethyl-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 5.00 mg (0.0106 mmol) of 4-(4,6-dimethoxy-1,3,5-triazin-2-yl)-4-methylmorpholinium chloride (DMT-MM) in 1 ml of ethanol was then added, 0.5 ml of ethanol and 2.5 ml of water were further added, and the resultant liquid was stirred at room temperature overnight. 1 ml of 20% sodium chloride aqueous solution and 30 ml of ethanol were added to the reaction solution to form precipitates and the supernatant of the suspension was removed. 14 mL of ethanol was further added and the supernatant was removed. Thereafter, washing with 90% ethanol was performed two times, washing with ethanol was performed two times, and washing with diethyl ether was further performed two times. The obtained precipitates were dried overnight using a vacuum pump to obtain 78 mg of the title compound. Based on the measurement result (247 nm) of a spectrophotometer, the introduction rate of ondansetron per total weight of the polymer conjugate was 3 wt %.

### Example 45

### 3,3-dimethyl-2-[[(ondansetron)methoxy]carbonyl]butyl]amino-alginic Acid Conjugate

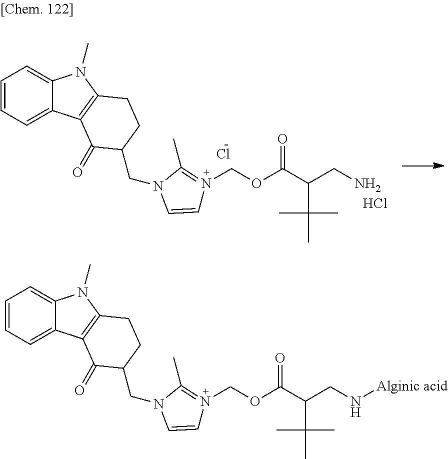

3 ml of water and 12 ml of ethanol were slowly added dropwise under stirring to 10 g (0.505 mmol) of 1% sodium alginate aqueous solution. To the mixed liquid, a solution of 6.6 mg (0.0126 mmol) of 3-[[2-(aminomethyl)-3,3-dimethyl-1-oxobutoxy]methyl]-2-methyl-1-[(2,3,4,9-tetrahydro-9-methyl-4-oxo-1H-carbazol-3-yl)methyl]-1H-imidazolium chloride hydrochloride in 1 ml of ethanol was added, a solution of 5.9 mg (0.0126 mmol) of 4-(4,6-dimethoxy-1,3,5-triazin-2-yl)-4-methylmorpholinium chloride (DMT-MM) in 1 ml of ethanol was then added, 1 ml of ethanol and 2 ml of water were further added, and the resultant liquid was stirred at room temperature overnight. 1.5 ml of 20% sodium chloride aqueous solution was added to the reaction solution and stirred. 200 ml of acetone was added to the reaction solution to form precipitates. Then, the supernatant of the suspension was removed. Thereafter, 90 mL of 90% acetone was added and the supernatant was removed. Thereafter, washing with 90% acetone was performed two times, washing with acetone was performed two times, and washing with diethyl ether was further performed two times. The obtained precipitates were dried overnight using a vacuum pump to obtain 84 mg of the title compound. Based on the measurement result (247 nm) of a spectrophotometer, the introduction rate of ondansetron per total weight of the polymer conjugate was 3 wt %/o.

### Reference Example 62

### 1-[2-[(4-chlorophenyl)phenyl methoxy]ethyl]-1-[[3-[[(1,1-dimethylethoxy)carbonyl]amino]-2,2-dimethyl-1-oxopropoxy]methyl]piperidinium Iodide

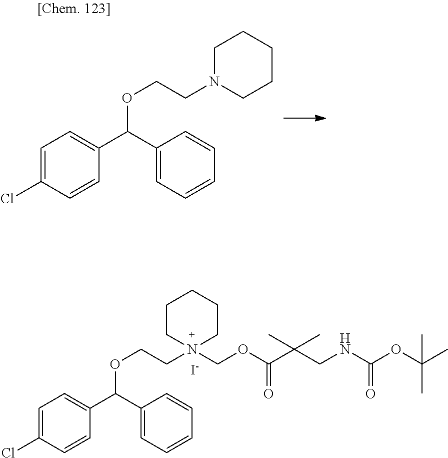

409 mg (1.24 mmol) of cloperastine was added at room temperature to a dichloromethane solution of 443 mg (1.24 mmol) of 3-[[(1,1-dimethylethoxy)carbonyl]amino]-2,2-dimethylpropane acid iodomethyl ester, and the reaction solution was condensed at 70° C. over 1 hour and then stirred at the same temperature for 2 hours. The residue was purified by silica gel column chromatography (2% methanol/chloroform) to obtain 668 mg (78%) of the title compound.

¹H-NMR(CDCl₃, δ):1.22(611, s), 1.34 (9H, s), 1.77-2.01 (6H, m), 3.25 (2H, d, J=7 Hz), 3.64-3.71 (2H, m), 3.98-3.99 (2H, m), 4.03-4.05 (2H, m), 4.15-4.16 (2H, m), 4.88(1H, br-s), 5.57 (1H, s), 5.59 (1H, d, J=9 Hz), 5.62 (1H, d, J=9 Hz), 7.26-7.36 (9H, m)

### Example 46

### 1-[(3-amino-2,2-dimethyl-1-oxopropoxy)methyl]-1-[2-[(4-chlorophenyl)phenyl methoxy]ethyl]-piperidinium Chloride Hydrochloride

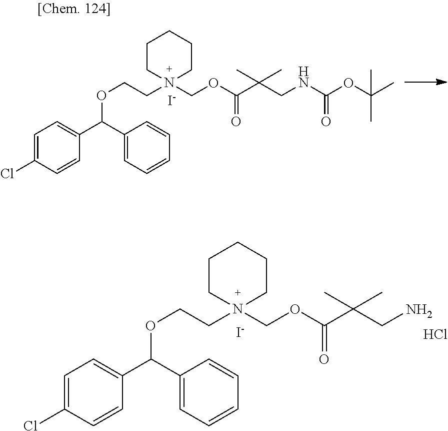

2 ml of 4 N hydrochloric acid/dioxane solution was added under cooling on ice to 2 ml of chloroform solution of 668 mg (0.97 mmol) of 1-[2-[(4-chlorophenyl)phenyl methoxy]ethyl]-1-[[3-[[(1,1-dimethylethoxy)carbonyl]amino]-2,2-dimethyl-1-oxopropoxy]methyl]piperidinium iodide. The reaction solution was warmed to room temperature and left to stand still for 1.5 hours. Thereafter, the solvent was distilled off under reduced pressure. The residue was dissolved in methanol and allowed to pass through 2 ml of Cl type ion exchange resin (DOWEX (registered trademark) 1×4 100-200 mesh), and the eluate was condensed under reduced pressure. Ethyl acetate was added to the residue and stirred for 2 hours. The precipitated crystals were filtered to obtain 365 mg (70%) of the title compound.

¹H-NMR (DMSO-d₆, δ):1.26 (3H, s), 1.33 (3H, s), 1.55-1.62 (2H, m), 1.82-1.91 (4H, m), 3.02-3.06 (2H, m), 3.42-3.64 (5H, m), 3.84-3.88 (3H, m), 5.47-5.49 (2H, m), 5.71-5.77 (1H, m), 7.27-7.51 (9H, m), 8.66(3H, br-s)

### Example 47

### [3-[(cloperastine)methoxy]-2,2-dimethyl-3-oxopropyl]amino-chondroitin Sulfate Conjugate

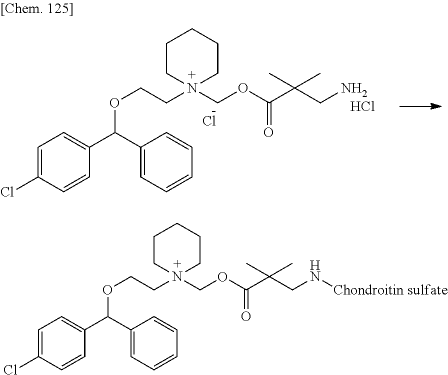

2 ml of ethanol was slowly added dropwise under stirring to 4.0 g (0.398 mmol) of 5% chondroitin sulfate aqueous solution (prepared by dissolving sodium chondroitin sulfate). To the mixed liquid, a solution of 43 mg (0.080 mmol) of 1-[(3-amino-2,2-dimethyl-1-oxopropoxy)methyl]-1-[2-[(4-chlorophenyl)phenyl methoxy]ethyl]-piperidinium chloridehydrochloride in 1 ml of ethanol was added, a solution of 38 mg (0.08 mmol) of 4-(4,6-dimethoxy-1,3,5-triazin-2-yl)-4-methylmorpholinium chloride (DMT-MM) in 1 ml of ethanol was then added, 1 ml of ethanol and 1 ml of water were further added, and the resultant liquid was stirred at room temperature overnight. 100 μl of 20% sodium chloride aqueous solution was added to the reaction solution, and ethanol was further added dropwise (2 mil) until immediately before the reaction solution became cloudy. The reaction solution was added dropwise under stirring to 8 ml of 90% ethanol, and 9 ml of ethanol was added to the mixed liquid and stirred for 1 hour. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 181 mg of the title compound. Based on values of integral in ¹H-NMR, the introduction rate of cloperastine per unit of whole disaccharide (glucuronic acid) of chondroitin sulfate was 7%.

### Example 48

### [2-ethyl-2-[[(ondansetron)methoxy]carbonyl]butyl]amino-polyglutamic Acid Conjugate

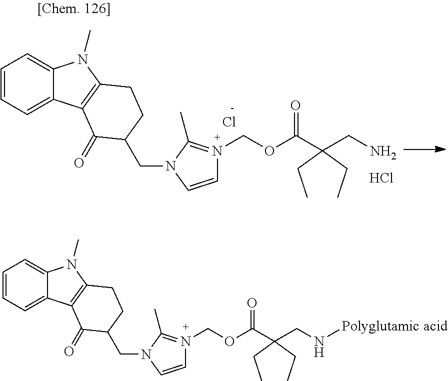

2 ml of ethanol was slowly added dropwise under stirring to 3.33 g (0.662 mmol) of 3% sodium polyglutamate aqueous solution. To the mixed liquid, a solution of 17.3 mg (0.033 mmol) of 3-[[2-(aminomethyl)-2-ethyl-1-oxobutoxy]methyl]-2-methyl-1-[(2,3,4,9-tetrahydro-9-m ethyl-4-oxo-1H-carbazol-3-yl)methyl]-1H-imidazolium chloride hydrochloride in 1 ml of ethanol was added, a solution of 15.2 mg (0.033 mmol) of 4-(4,6-dimethoxy-1,3,5-triazin-2-yl)-4-methylmorpholinium chloride (DMT-MM) in 1 ml of ethanol was then added, 3 ml of ethanol and 3.7 ml of water were further added, and the resultant liquid was stirred at room temperature overnight. 100 μl of 20% sodium chloride aqueous solution was added to the reaction solution, and ethanol was further added dropwise (4 ml) until immediately before the reaction solution became cloudy. The reaction solution was added dropwise under stirring to 10 ml of 90% ethanol, and 4 ml of ethanol was added to the mixed liquid and stirred for 2 hours. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 62 mg of the title compound. Based on the measurement result (247 nm) of a spectrophotometer, the introduction rate of ondansetron per total weight of the polymer conjugate was 6 wt %.

### Example 49

### [2-ethyl-2-[[(ondansetron)methoxy]carbonyl]butyl]amino-polyacrylic Acid Conjugate

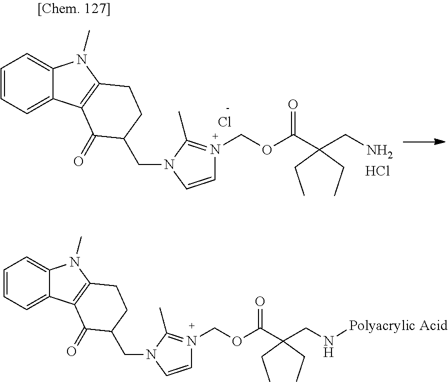

3 ml of ethanol was slowly added dropwise under stirring to 5 g (1.06 mmol) of 2% sodium polyacrylate aqueous solution. To the mixed liquid, a solution of 14.1 mg (0.027 mmol) of 3-[[2-(aminomethyl)-2-ethyl-1-oxobutoxy]methyl]-2-methyl-1-[(2,3,4,9-tetrahydro-9-m ethyl-4-oxo-1H-carbazol-3-yl)methyl]-1H-imidazolium chloride hydrochloride in 1 ml of ethanol was added, a solution of 12.2 mg (0.027 mmol) of 4-(4,6-dimethoxy-1,3,5-triazin-2-yl)-4-methylmorpholinium chloride (DMT-MM) in 1 ml of ethanol was then added, 3 ml of ethanol and 5 ml of water were further added, and the resultant liquid was stirred at room temperature overnight. The reaction solution was condensed, ethanol was distilled off, and then freeze dry was performed. The obtained solid substance was washed with 90% ethanol two times, with 90% ethanol two times, with ethanol two times, and further with diethyl ether two times. The obtained solid substance was dried overnight using a vacuum pump to obtain 65 mg of the title compound. Based on the measurement result (247 nm) of a spectrophotometer, the introduction rate of ondansetron per total weight of the polymer conjugate was 6 wt %.

### Test Example 1 Drug Release Test of Drug-Polymer Conjugate

### [Operation]

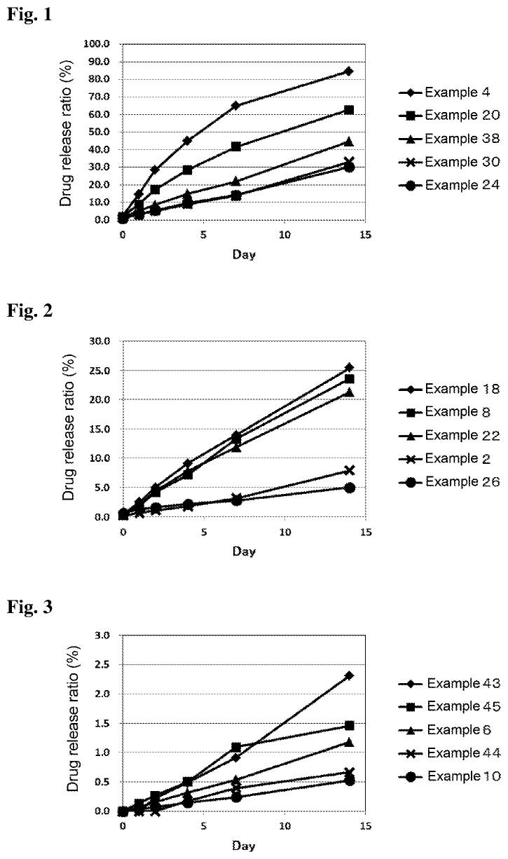

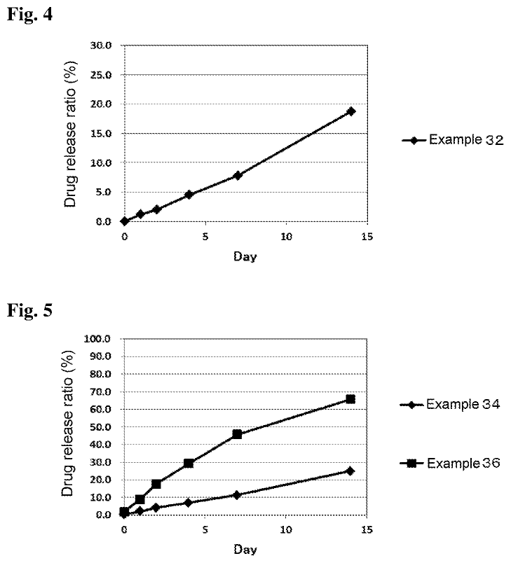

Each evaluation polymer conjugate presented in Table 1 was dissolved in a concentration of 1.5 mg/ml in a sodium phosphate buffer solution having a pH of 7.0 and then dispensed. Immediately after dissolving, the drug-polymer conjugate present in the solution and the release drug amount were analyzed as an initial state (storage 0 day) by SEC-HPLC. Other divided injection liquid was stored under the condition of 36° C. immediately after dissolving, and the drug amount after each time elapsed was analyzed in the similar manner. From a ratio of the release drug amount and the drug-polymer conjugate amount at each time point which had been obtained in this way, a drug release ratio (%) was calculated. A relation between time and the drug release ratio is as shown in FIGS. 1 to 5.

The HPLC conditions are as follows.

Column: TSGgel α-3000 (7.8 mm×300 mm)

Flow rate: 0.5 mL/min

Temperature: 35° C.

Mobile phase: acetonitrile/physiological saline solution=1/2

TABLE 1
[Evaluation polymer conjugate]

| Example | | |
|---|---|---|
| No. | Compound name | Structure |
| Example 4 | [3-[(ondansetron)methoxy]-3-oxopropyl]amino- chondroitin sulfate conjugate | 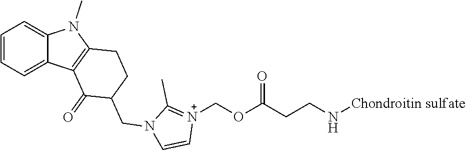 |
| Example 20 | [2-methyl-3-[[(ondansetron)methoxy]-3- oxopropyl]amino-chondroitin sulfate conjugate | 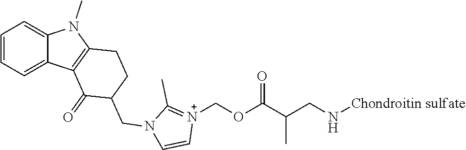 |
| Example 38 | [3-phenyl-2- [[(ondansetron)methoxy]carbonyl]propyl]amino- chondroitin sulfate conjugate | 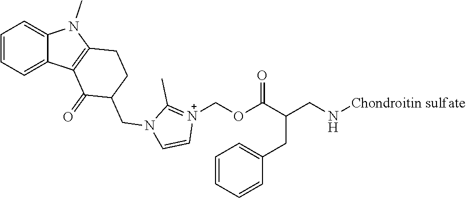 |
| Example 30 | [2-[[(ondansetron)methoxy]carbonyl]butyl]amino- chondroitin sulfate conjugate | 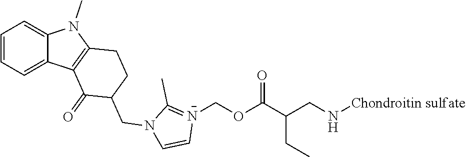 |
| Example 24 | [1- [[(ondansetron)methoxy]carbonyl]cyclopropyl] amino-chondroitin sulfate conjugate | 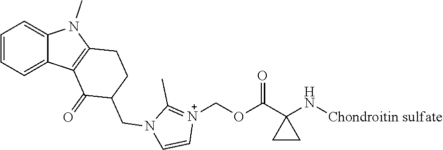 |
| Example 18 | [[1- [[(ondansetron)methoxy]carbonyl]cyclopentyl] methyl]amino-chondroitin sulfate conjugate | 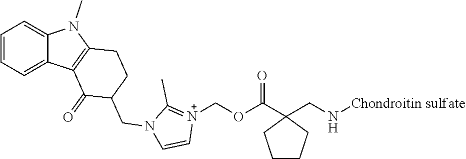 |
| Example 8 | [[1- [[(ondansetron)methoxy]carbonyl]cyclopropyl] methyl]amino-chondroitin sulfate conjugate | 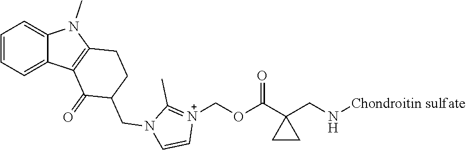 |
| Example 22 | [(S)-2,2-dimethyl-1- [[(ondansetron)methoxy]carbonyl]propyl]amino- chondroitin sulfate conjugate | 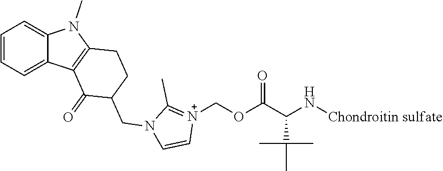 |
| Example 2 | [3-methyl-2- [[(ondansetron)methoxy]carbonyl]butyl]amino- chondroitin sulfate conjugate | 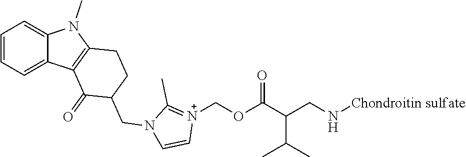 |
| Example 26 | [3-methyl-2-[[1- (ondansetron)ethoxy]carbonyl]butyl]amino- chondroitin sulfate conjugate | 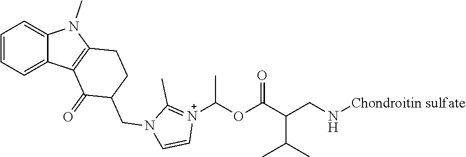 |
| Example 43 | [3,3-dimethyl-2- [[(ondansetron)methoxy]carbonyl]butyl]amino- hyaluronic acid conjugate | 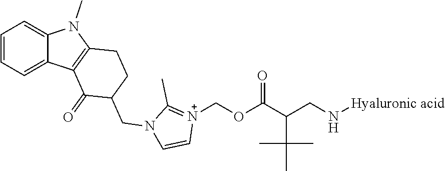 |
| Example 45 | [3,3-dimethyl-2- [[(ondansetron)methoxy]carbonyl]butyl]amino- alginic acid conjugate | 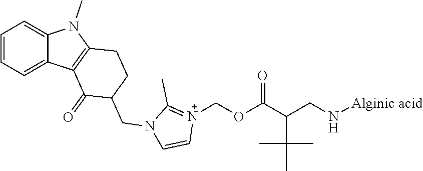 |
| Example 6 | [2-ethyl-2- [[(ondansetron)methoxy]carbonyl]butyl]amino- chondroitin sulfate conjugate | 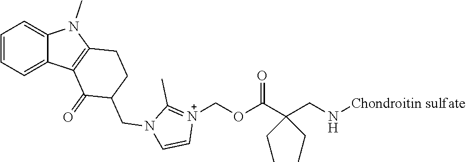 |
| Example 44 | [3,3-dimethyl-2- [[(ondansetron)methoxy]carbonyl]butyl]amino- carboxymethyl cellulose conjugate | 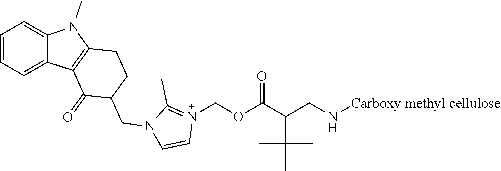 |
| Example 10 | [3,3-dimethyl-2- [[(ondansetron)methoxy]carbonyl]butyl]amino- chondroitin sulfate conjugate | 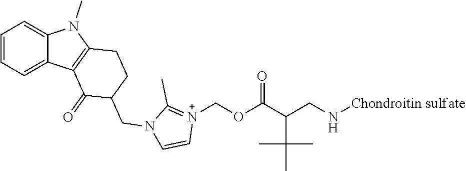 |
| Example 32 | [2-[[(cloperastine)methoxy]carbonyl]-3- methylbutyl]amino-chondroitin sulfate conjugate | 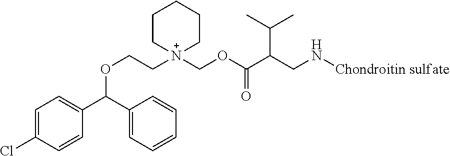 |
| Example 34 | [3-methyl-2- [[(promethazine)methoxy]carbonyl]butyl]amino- chondroitin sulfate conjugate | 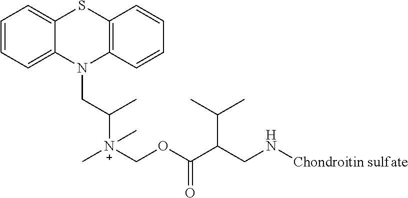 |
| Example 36 | [2-methyl-2- [[(promethazine)methoxy]carbonyl]propyl]amino- chondtroitin sulfate conjugate | 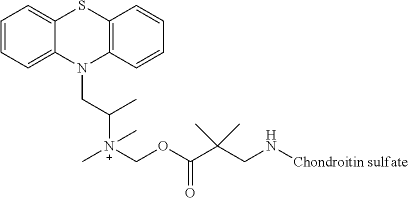 |

As shown in FIGS. 1 to 5, the conjugate of the present invention enables releasing of various tertiary amine-based drugs to be performed which starts in hydrolysis, and the release rate thereof can also be adjusted by the structure of the linker.

The present invention includes inventions specified by the following items. 1. A compound represented by Formula (I);

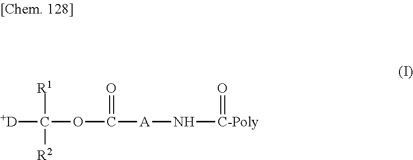

in the formula, D⁺ is a structure forming a quaternary ammonium salt or an iminium salt formed from D, a tertiary amine-type compound or imine-type compound; R¹ bonds to a carbon atom to be bonded on a nitrogen atom forming the quaternary ammonium salt or the iminium salt; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a bivalent hydrocarbon group in which a carbon other than carbons at the terminal may be substituted with a hetero atom selected from the group consisting of an oxygen atom, a nitrogen atom, and a sulfur atom; R¹ and R² may combine together with both substituents or a partial structure of A to form a ring, and Poly is a polymer residue having a carboxy group.

2. A compound represented by Formula (II);

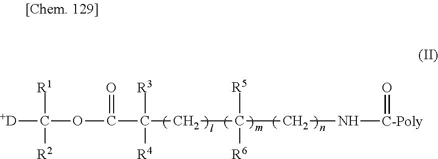

in the formula, D⁺, R¹, R², and Poly are as defined above; R³, R⁴, R⁵ and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three substituents of R¹, R², R³, R⁴, R⁵ and R⁶ may combine together to form a ring; l and n are each independently 0, 1, or 2, and m is 0 or 1.

3. The compound according to 1. or 2, wherein in Formula (I) or (II); R¹, R², R³, R⁴, R⁵ and R⁶ are each independently is a hydrogen atom; a substituted or unsubstituted linear or branched chain alkyl group having carbon number of 1 to 6; a substituted or unsubstituted cycloalkyl group having carbon number of 3 to 8; a substituted or unsubstituted linear or branched alkenyl group having carbon number of 2 to 6; a substituted or unsubstituted cycloalkenyl group having carbon number of 3 to 8; a substituted or unsubstituted linear or branched alkynyl group having carbon number of 2 to 6; a substituted or unsubstituted monocyclic or polycyclic aromatic group having carbon number of 6 to 14; or a substituted or unsubstituted 3- to 8-membered heterocyclic group containing at least one of a nitrogen atom, an oxygen atom, or a sulfur atom as a ring-constituting atom.
4. The compound according to any one of 1. to 3, wherein in Formula (I) or (II); a substituent of alkyl, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁶ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group (herein, Rx, Ry, and Rz each independently represent a hydrogen atom, an alkyl group, a cycloalkyl group, an alkenyl group, a cycloalkenyl group, an alkynyl group, an aromatic hydrocarbon group, or a heterocyclic group; in addition, Rx, Ry, and Rz may be bonded to each other to form a saturated or unsaturated hetero ring, the ring is also capable of forming a condensed ring or a spiro ring with an aliphatic ring or a hetero ring, and also capable of forming a condensed ring with an aromatic ring).
5. The compound according to any one of 1. to 4, in which in Formula (I) or (II), Poly is a water-soluble polymer residue.
6. The compound according to any one of 1. to 4, wherein Formula (I) or (II), Poly is a polysaccharide residue.
7. The compound according to any one of 1. to 4, wherein in Formula (I) or (II), Poly is a glycosaminoglycan residue.
8. The compound according to any one of 1. to 4, wherein in Formula (I) or (II), Poly is a residue of chondroitin, chondroitin sulfate or hyaluronic acid.
9. A method for producing a compound represented by the following Formula (I), the method including a step of condensing a compound represented by the following Formula (III) and a polymer having a carboxy group represented by the following Formula (IV):

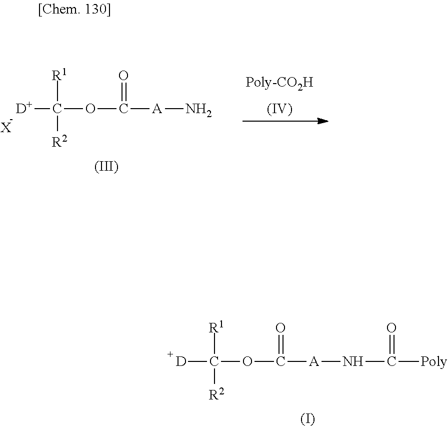

wherein, D⁺, A, and Poly in the above (I), (II), and (IV) are as defined above; X⁻ is a counter anion of the quaternary ammonium salt or the iminium salt, and (III) may form a salt with an inorganic acid or an organic acid.

10. A linker represented by the following Formula (V) for bonding a tertiary amine-type compound containing a nitrogen atom capable of forming a quaternary ammonium salt or an imine-type compound capable of forming an iminium salt to a polymer having a carboxy group:

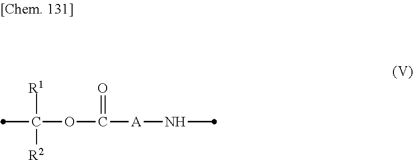

wherein, R¹, R², and A in the above (V) are as defined above; regarding ● at both ends, the left side is a node with a quaternary ammonium salt or an iminium salt, and the right side represents a node with carbonyl condensed with a polymer having a carboxy group.

11. A method for producing the compound represented by Formula (I) according to 1, the method including a step of bonding a tertiary amine-type compound containing a nitrogen atom capable of forming a quaternary ammonium salt or an imine-type compound capable of forming an iminium salt to a polymer having a carboxy group via the linker according to 10.

The disclosure of Japanese Patent Application No. 2017-086223 (filing date: Apr. 25, 2017) is incorporated into the present specification by Reference.

1. A compound represented by Formula (I) or a pharmaceutically acceptable salt thereof; 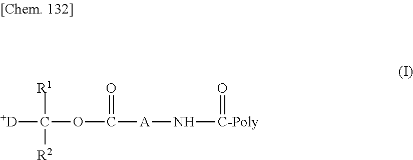
in Formula (I), D⁺ is a structure forming a quaternary ammonium salt or an iminium salt from D, a tertiary amine compound or an imine compound; a nitrogen atom forming the quaternary ammonium salt or the iminium salt and a carbon atom to which R¹ and R² bond are bonded to each other, R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a bivalent hydrocarbon group in which a carbon other than carbons at both ends may be substituted with a hetero atom selected from the group consisting of an oxygen atom, a nitrogen atom and a sulfur atom; any two or three groups of R¹, R², and A may combine together to form a ring; and Poly is a polymer residue having a carboxy group.

2. A compound represented by Formula (II) or a pharmaceutically acceptable salt thereof; 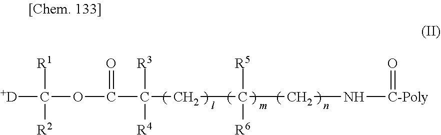
in Formula (II), D⁺, R¹, R², and Poly are as defined in claim 1; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring; 1 and n are each independently 0, 1, or 2; and m is 0 or 1.

3. The compound according to claim 1 or a pharmaceutically acceptable salt thereof, wherein in Formula (I) or (II); R¹, R², R³, R⁴, R⁵, and R⁶ are each independently a hydrogen atom; a substituted or unsubstituted linear or branched chain alkyl group having carbon number of 1 to 6; a substituted or unsubstituted cycloalkyl group having carbon number of 3 to 8; a substituted or unsubstituted linear or branched alkenyl group having carbon number of 2 to 6; a substituted or unsubstituted cycloalkenyl group having carbon number of 3 to 8; a substituted or unsubstituted linear or branched alkynyl group having carbon number of 2 to 6; a substituted or unsubstituted monocyclic or polycyclic aromatic group having carbon number of 6 to 14; or a substituted or unsubstituted 3- to 8-membered heterocyclic group containing at least one of a nitrogen atom, an oxygen atom, or a sulfur atom as a ring-constituting atom.

4. The compound according to claim 1 or a pharmaceutically acceptable salt thereof, wherein in Formula (I) or (II), a substituent of alkyl, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁶ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group, Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, and a heterocyclic group, and at this time, two or more of Rx, Ry, and Rz may be combined together to form a saturated or unsaturated hetero ring.

5. The compound according to claim 1 or a pharmaceutically acceptable salt thereof, wherein in Formula (I) or (II), Poly is a water-soluble polymer residue.

6. The compound according to claim 1 or a pharmaceutically acceptable salt thereof, wherein in Formula (I) or (II), Poly is a polysaccharide residue.

7. The compound according to claim 1 or a pharmaceutically acceptable salt thereof, wherein in Formula (I) or (II), Poly is a glycosaminoglycan residue.

8. The compound according to claim 1 or a pharmaceutically acceptable salt thereof, wherein in Formula (I) or (II), Poly is a residue of chondroitin, chondroitin sulfate or hyaluronic acid.

9. A method for producing a compound represented by the following Formula (I) or a pharmaceutically acceptable salt thereof, the method comprising a step of condensing a compound represented by the following Formula (III) and a polymer having a carboxy group represented by the following Formula (IV): 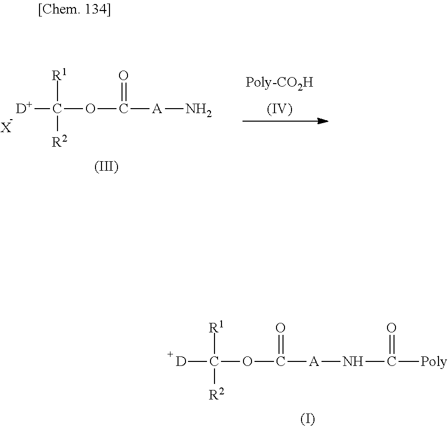
in Formulae (I), (III), and (IV), D⁺, A, R¹, R², and Poly are as defined in claim 1; X⁻ is a counter anion of D⁺; and the compound represented by Formula (III) may form a salt with an inorganic acid or an organic acid.

10. The production method according to claim 9, wherein the compound represented by the Formula (III) is a compound represented by the following Formula (IX), and the compound represented by the Formula (I) is a compound represented by the following Formula (II): 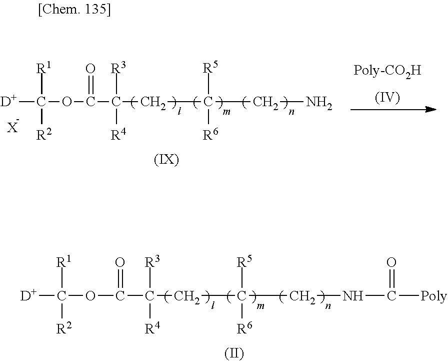
in Formulae (II), (IV), and (IX), D⁺ is a structure forming a quaternary ammonium salt or an iminium salt from D⁺ a tertiary amine compound or an imine compound: a nitrogen atom forming the quaternary ammonium salt or the iminium salt and a carbon atom to which R¹ and R² bond are bonded to each other, R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group: Poly is a polymer residue having a carboxy group; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring: l and n are each independently 0, 1, or 2; and m is 0 or 1; X⁻ is a counter anion of D⁺; and the compound represented by Formula (IX) may form a salt with an inorganic acid or an organic acid.

11. A linker represented by the following Formula (V) for bonding a tertiary amine compound containing a nitrogen atom capable of forming a quaternary ammonium salt or an imine compound capable of forming an iminium salt to a polymer having a carboxy group: 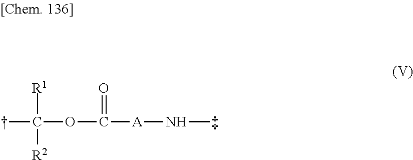
wherein, R¹, R², and A in the (V) are as defined in claim 1, symbol † is a node with the nitrogen atom forming the quaternary ammonium salt or the iminium salt, and symbol ‡ represents a node with a moiety of the carboxy group excluding a hydroxyl group of the polymer having a carboxy group.

12. The linker according to claim 11, wherein the linker is represented by the following Formula (XV): 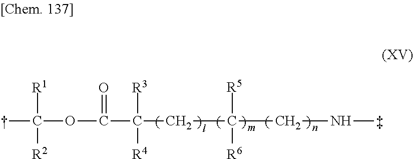
wherein, D⁺ is a structure forming a quaternary ammonium salt or an iminium salt from D⁺ a tertiary amine compound or an imine compound: a nitrogen atom forming the quaternary ammonium salt or the iminium salt and a carbon atom to which R¹ and R² bond are bonded to each other, R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic grouD; any two or three groups of R¹, R², R³, R⁴, R⁵, and R⁶ may combine together to form a ring: l and n are each independently 0, 1, or 2; and m is 0 or 1, symbol † is a node with the nitrogen atom forming the quaternary ammonium salt or the iminium salt, and symbol ‡ represents a node with a moiety of the carboxy group excluding a hydroxyl group of the polymer having a carboxy group.

13. A method for producing a conjugate, the method comprising a step of bonding a tertiary amine compound containing a nitrogen atom capable of forming a quaternary ammonium salt or an imine compound capable of forming an iminium salt to a polymer having a carboxy group via the linker according to claim 11.
